(19) United States
(12) Patent Application Publication
(10) Pub. No.: US 2026/0209357 A1
(43) Pub. Date: Jul. 23, 2026

(54) FcRc Antagonists and Uses Thereof

(71) Applicant: CSL Innovation PTY LTD, Melbourne, Victoria (AU)

(72) Inventors: Anne Verhagen, Melbourne (AU); Rodrigo Morales, Melbourne (AU); Steve Dower, Melbourne (AU)

(73) Assignee: CSL Innovation PTY LTD, Melbourne, Victoria (AU)

(21) Appl. No.: 19/139,812

(22) PCT Filed: Dec. 18, 2023
(86) PCT No.: PCT/AU23/51319

§ 371 Date: Jun. 16, 2025

(30) Foreign Application Priority Data
Dec. 20, 2022 (AU) 2022903917

(51) Int. Cl. C07K 16/28; A61K 38/00; A61K 39/00; A61P 37/06; C07K 14/765

(52) U.S. Cl. CPC C07K 16/283; A61P 37/06; C07K 14/765; A61K 38/00; A61K 2039/505; C07K 2317/52; C07K 2317/76; C07K 2317/92; C07K 2319/31

(57) ABSTRACT

The present disclosure relates to antagonists of FcRn having an extended half-life comprising an immunoglobulin Fc domain and at least one albumin.

### RELATED APPLICATION DATA

The present application claims priority from Australian Patent Application No. 2022903917 filed on 20 Dec. 2022 entitled “FcRn ANTAGONISTS AND USES THEREOF”. The entire contents of which is hereby incorporated by reference.

### SEQUENCE LISTING

The present application is filed with a Sequence Listing in electronic form. The entire contents of the Sequence Listing are hereby incorporated by reference.

### FIELD

The present disclosure relates to antagonists of FcRn having an extended half-life.

### BACKGROUND

Immunoglobulin gamma (IgG) plays a key role in the pathology of many disorders, such as autoimmune diseases, inflammatory diseases, and disorders in which the pathology is characterized by over-expression of IgG (e.g., hypergammaglobulinemia) (see e.g. Junghans, Immunol Res. 16:29 (1997)).

The half-life of IgG in the serum is prolonged relative to the serum half-life of other plasma proteins. This long half-life is due, in part, to the binding of the Fc domain of IgG to the neonatal Fc receptor (FcRn). In adults, FcRn functions to protect IgG from degradation. FcRn binds to pinocytosed IgG and protects the IgG from transport to degradative lysosomes by recycling it back to the extracellular compartment. This recycling is facilitated by the pH-dependent binding of IgG to FcRn, where the IgG/FcRn interaction is stronger at acidic endosomal pH than at extracellular physiological pH.

When the serum concentration of IgG reaches a level that exceeds available FcRn molecules, unbound IgG is not protected from degradative mechanisms and will consequently have a reduced serum half-life. Thus, inhibition of IgG binding to FcRn reduces the serum half-life of IgG by preventing IgG endosomal recycling of IgG. Accordingly, agents that antagonize the binding of IgG to FcRn are useful for regulating, treating or preventing antibody-mediated disorders, such as autoimmune diseases.

Currently, some autoimmune diseases are treated by an intravenous infusion of pooled IgG (IVIg) from human donors. As many of these autoimmune diseases are chronic, afflicted individuals may require repeated administrations of IVIg and/or other suitable therapies in order to manage their disease.

In another approach, blocking antibodies to FcRn have been developed to inhibit IgG binding to FcRn. Peptides have also been identified that bind to and antagonize FcRn function.

Recently, a FcRn inhibitor has been developed, which is a modified version of human IgG1 Fc domain (efgartigimod). This compound has been approved for treatment of myasthenia gravis (MG). A disadvantage of this compound is its relatively short half-life, requiring regular infusions for effective treatment.

### SUMMARY

In work leading up to the present disclosure, the inventors recognized the relatively short half-life of FcRn antagonists, such as efgartigimod, as a limitation of the molecule and a potential problem for patient compliance and sought to produce FcRn antagonists having extended half-lives. The inventors conjugated a Fc domain of an immunoglobulin, e.g., an IgG1, to an albumin to extend the half-life of the resulting FcRn antagonist. Unexpectedly, the inventors found that linking the C-terminus of the Fc domain to the N-terminus of the albumin resulted in a longer half-life and the resulting FcRn antagonist more potently inhibited FcRn than in the opposite orientation, i.e. linking the N-terminus of the Fc to the C-terminus of the albumin. This finding can be considered counter-intuitive since most Fc fusion proteins comprise the fusion partner at the N-terminal end of the protein, i.e., away from the stable structure formed by the Fc C_{H}3 domain. A FcRn antagonist comprising a Fc domain having its C-terminus fused to the N-terminus of the albumin resulted in a longer half-life than efigartgimod.

The inventors extended their studies using variants of albumin that have reduced affinity for FcRn or that do not bind to FcRn. The inventors hypothesised that using a wild-type albumin in the antagonist may affect endogenous albumin recycling via FcRn and there may be a risk of dyslipidemia or increased cholesterol known to be associated with impaired albumin homeostasis and hypoalbuminemia. On the other hand, a potential disadvantage of using an albumin that has reduced affinity for FcRn or that does not bind to FcRn is that it may not extend the half-life of the antagonist to the same extent. The inventors demonstrated that a FcRn antagonist comprising an albumin having reduced or no binding to FcRn retained its ability to inhibit binding of IgG to FcRn and retained its extended half-life.

The inventors additionally showed that the FcRn antagonist can be administered subcutaneously and reduce circulating IgG levels.

Based on the foregoing, the present disclosure provides a FcRn antagonist comprising an Fc domain from an immunoglobulin or fragment thereof and an albumin or fragment thereof.

For example, the present disclosure provides a FcRn antagonist comprising:
(i) an immunoglobulin Fc domain or fragment thereof capable of binding to a FcRn; and
(ii) at least one albumin or fragment thereof capable of extending the half-life of the FcRn antagonist compared to the half-life of the immunoglobulin Fc domain or the fragment thereof.

A FcRn antagonist of the disclosure inhibits or reduces binding of immunoglobulins, e.g., IgG, to a FcRn. For example, administration of the FcRn antagonist of the disclosure to a subject inhibits or reduces binding of immunoglobulin, e.g., IgG, to FcRn. Such inhibition can be determined using standard methods known in the art, for example, by administering an antibody (a ‘tracer antibody’) to the subject and measuring clearance of the tracer antibody from circulation, wherein increased clearance and/or more rapid clearance of the tracer antibody from circulation compared to the level or rate of clearance in a control subject is indicative of a FcRn antagonist.

In the present disclosure, the Fc domain or fragment thereof of the FcRn antagonist is not an immunoglobulin. For example, the FcRn antagonist of the disclosure comprises only the Fc domain of an immunoglobulin or comprises the Fc domain and the hinge region but not the C_{H}1, C_{L} and variable domains.

In one example, the present disclosure provides a FcRn antagonist comprising:
(i) a region of an immunoglobulin consisting of the Fc domain or fragment thereof capable of binding to a FcRn; and
(ii) at least one albumin or fragment thereof capable of extending the half-life of the FcRn antagonist compared to the half-life of the immunoglobulin Fc or the fragment thereof.

In one example, the present disclosure provides a FcRn antagonist consisting of:
(i) an immunoglobulin Fc domain or fragment thereof capable of binding to a FcRn;
(ii) at least one albumin or fragment thereof capable of extending the half-life of the FcRn antagonist compared to the half-life of the immunoglobulin Fc domain or the fragment thereof; and
(iii) optionally, a linker positioned between (i) and (ii).

In one example, the FcRn antagonist has a longer serum half-life compared to the serum half-life of the immunoglobulin Fc domain or fragment thereof alone. For example, the FcRn antagonist has a half-life that is at least about two times or three times or four times or five times longer compared to the half-life of the immunoglobulin Fc domain or fragment thereof alone. For example, the FcRn antagonist has a half-life that is at least about two times longer compared to the half-life of the immunoglobulin Fc domain or fragment thereof alone. For example, the FcRn antagonist has a half-life that is at least about three times longer compared to the half-life of the immunoglobulin Fc domain or fragment thereof alone. For example, the FcRn antagonist has a half-life that is at least about four times longer compared to the half-life of the immunoglobulin Fc domain or fragment thereof alone. For example, the FcRn antagonist has a half-life that is at least about five times longer compared to the half-life of the immunoglobulin Fc domain or fragment thereof alone.

In one example, the FcRn antagonist has a half-life that is at least 25 hours, 30 hours, 35 hours, 40 hours, 45 hours, 50 hours, 55 hours, 60 hours, 65 hours, 70 hours, 75 hours or 80 hours longer compared to the half-life of the immunoglobulin Fc domain or fragment thereof alone. In one example, the FcRn antagonist has a half-life that is at least 25 hours longer compared to the half-life of the immunoglobulin Fc domain or fragment thereof alone. In one example, the FcRn antagonist has a half-life that is at least 30 hours longer compared to the half-life of the immunoglobulin Fc domain or fragment thereof alone. In one example, the FcRn antagonist has a half-life that is at least 35 hours longer compared to the half-life of the immunoglobulin Fc domain or fragment thereof alone. In one example, the FcRn antagonist has a half-life that is at least 40 hours longer compared to the half-life of the immunoglobulin Fc domain or fragment thereof alone. In one example, the FcRn antagonist has a half-life that is at least 45 hours longer compared to the half-life of the immunoglobulin Fc domain or fragment thereof alone. In one example, the FcRn antagonist has a half-life that is at least 50 hours longer compared to the half-life of the immunoglobulin Fc domain or fragment thereof alone. In one example, the FcRn antagonist has a half-life that is at least 55 hours longer compared to the half-life of the immunoglobulin Fc domain or fragment thereof alone. In one example, the FcRn antagonist has a half-life that is at least 60 hours longer compared to the half-life of the immunoglobulin Fc domain or fragment thereof alone. In one example, the FcRn antagonist has a half-life that is at least 65 hours longer compared to the half-life of the immunoglobulin Fc domain or fragment thereof alone. In one example, the FcRn antagonist has a half-life that is at least 70 hours longer compared to the half-life of the immunoglobulin Fc domain or fragment thereof alone. In one example, the FcRn antagonist has a half-life that is at least 75 hours longer compared to the half-life of the immunoglobulin Fc domain or fragment thereof alone. In one example, the FcRn antagonist has a half-life that is at least 80 hours longer compared to the half-life of the immunoglobulin Fc domain or fragment thereof alone.

In one example, the antagonist binds to human FcRn at neutral pH with an affinity constant (K_{D}) of at least 700 nM and/or to human FcRn at acidic pH with a K_{D} of at least 100 nM, wherein the antagonist is immobilised on a solid support and the binding of soluble human FcRn to the antagonist is determined using surface plasmon resonance (SPR). In one example, the antagonist binds to human FcRn at neutral pH with a K_{D} of at least 650 nM or 600 nM and/or to human FcRn at acidic pH with a K_{D} of at least 80 nM or 70 nM or 65 nM, wherein the antagonist is immobilised on a solid support and the binding of soluble human FcRn to the antagonist is determined using SPR. In one example, the antagonist binds to human FcRn at neutral pH with a K_{D} of at least 600 nM and/or to human FcRn at acidic pH with a K_{D} of at least 65 nM, wherein the antagonist is immobilised on a solid support and the binding of soluble human FcRn to the antagonist is determined using SPR.

For the purposes of clarification and as will be apparent to the skilled artisan based on the description herein, reference to an “affinity of at least about” or “at least” will be understood to mean that the affinity is equal to the recited value or higher (i.e., the value recited as the affinity is lower), i.e., an affinity of 2 nM is greater than an affinity of 3 nM. Stated another way, this term could be “an affinity of X or less” or “an affinity of no more than X”, wherein X is a value recited herein.

In one example, the antagonist binds to cynomolgus monkey FcRn at neutral pH with a K_{D} of at least 500 nM and/or to cynomolgus monkey FcRn at acidic pH with a K_{D} of at least 100 nM, wherein the antagonist is immobilised on a solid support and the binding of soluble cynomolgus monkey FcRn to the antagonist is determined using surface plasmon resonance (SPR).

In one example, the antagonist binds to mouse FcRn at neutral pH with a K_{D} of at least 20 nM and/or to mouse FcRn at acidic pH with a K_{D} of at least 1 nM, wherein the antagonist is immobilised on a solid support and the binding of soluble mouse FcRn to the antagonist is determined using surface plasmon resonance (SPR).

In one example, the antagonist binds to rat FcRn at neutral pH with a K_{D} of at least 160 nM and/or to rat FcRn at acidic pH with a K_{D} of at least 2 nM, wherein the antagonist is immobilised on a solid support and the binding of soluble rat FcRn to the antagonist is determined using surface plasmon resonance (SPR).

In one example, the antagonist binds to human FcRn at neutral pH with a K_{D} of at least 10 nM, wherein the FcRn is immobilised on a solid support and the binding of FcRn antagonist to the FcRn is determined using surface plasmon resonance (SPR). In one example, the antagonist binds to human FcRn at neutral pH with a K_{D} of at least 5 nM or 4 nM or 3 nM, wherein the FcRn is immobilised on a solid support and the binding of FcRn antagonist to the FcRn is determined using SPR. In one example, the antagonist binds to human FcRn at neutral pH with a K_{D} of at least 2.5 nM, wherein the FcRn is immobilised on a solid support and the binding of FcRn antagonist to the FcRn is determined using SPR.

In one example, “neutral pH” is about pH 7.3. In one example, “acidic pH” is about pH 6.

In one example, the binding of the antagonist to the FcRn or K_{D} is conferred by the immunoglobulin Fc domain or fragment thereof.

In an example of the present disclosure, the albumin or fragment thereof is a human albumin variant or fragment thereof. In one example, the human albumin variant or fragment thereof is a naturally-occurring albumin variant. The skilled person will recognize that in the situation of an albumin variant the reference to a “position corresponding to” a recited amino acid residue may need to be adjusted for any insertion or deletion.

For example, the albumin variant or fragment thereof binds with reduced affinity to FcRn compared to a albumin set forth in SEQ ID NO: 1. For example, the binding affinity is measured at neutral and/or acidic pH.

In one example, the albumin variant or fragment thereof binds to human FcRn at neutral pH or pH 6.0 with a K_{D} of greater than 5 μM or 7.5 μM or 10 μM. In one example, the albumin variant or fragment thereof binds to human FcRn at neutral pH or pH 6.0 with a K_{D} of greater than 10 μM.

In one example, the albumin variant or fragment thereof does not detectably bind to FcRn on the surface of HEK-293 cells expressing FcRn.

In one example, the albumin variant or fragment thereof comprises an amino acid substitution at a position corresponding to amino acid 464 of SEQ ID NO: 1.

In one example, the albumin comprises an amino acid substitution at a position corresponding to amino acid 391 of SEQ ID NO: 1.

In one example, the albumin comprises an amino acid substitution at a position corresponding to amino acid 402 of SEQ ID NO: 1.

In one example, the albumin comprises an amino acid substitution at a position corresponding to amino acid 418 of SEQ ID NO: 1.

In one example, the albumin comprises an amino acid substitution at a position corresponding to amino acid 420 of SEQ ID NO: 1.

In one example, the albumin comprises an amino acid substitution at a position corresponding to amino acid 424 of SEQ ID NO: 1.

In one example, the albumin comprises an amino acid substitution at a position corresponding to amino acid 429 of SEQ ID NO: 1.

In one example, the albumin comprises an amino acid substitution at a position corresponding to amino acid 462 of SEQ ID NO: 1.

In one example, the albumin comprises an amino acid substitution at a position corresponding to amino acid 492 of SEQ ID NO: 1.

In one example, the albumin comprises an amino acid substitution at a position corresponding to amino acid 501 of SEQ ID NO: 1.

In one example, the albumin comprises an amino acid substitution at a position corresponding to amino acid 545 of SEQ ID NO: 1.

In one example, the albumin comprises an amino acid substitution at a position corresponding to amino acid 547 of SEQ ID NO: 1.

In one example, the albumin comprises an amino acid substitution at a position corresponding to amino acid 422 of SEQ ID NO: 1.

In one example, the albumin comprises an amino acid substitution at a position corresponding to amino acid 510 of SEQ ID NO: 1.

In one example, the albumin comprises an amino acid substitution at a position corresponding to amino acid 535 of SEQ ID NO: 1.

In one example, the albumin comprises an amino acid substitution at a position corresponding to amino acid 463 of SEQ ID NO: 1.

In one example, the albumin comprises an amino acid substitution at a position corresponding to amino acid 467 of SEQ ID NO: 1.

In one example, the albumin comprises an amino acid substitution at a position corresponding to amino acid 505 of SEQ ID NO: 1.

In one example, the albumin comprises an amino acid substitution at a position corresponding to amino acid 509 of SEQ ID NO: 1.

In one example, the albumin comprises an amino acid substitution at a position corresponding to amino acid 519 of SEQ ID NO: 1.

An exemplary albumin variant or fragment thereof comprises one or more amino acid substitutions selected from the group consisting of:
(i) aspartic acid substituted for asparagine at a position corresponding to amino acid 391 of SEQ ID NO: 1;
(ii) glutamic acid substituted for lysine at a position corresponding to amino acid 402 of SEQ ID NO: 1;
(iii) methionine substituted for valine at a position corresponding to amino acid 418 of SEQ ID NO: 1;
(iv) alanine substituted for threonine at a position corresponding to amino acid 420 of SEQ ID NO: 1;
(v) isoleucine substituted for isoleucine at a position corresponding to amino acid 424 of SEQ ID NO: 1;
(vi) aspartic acid substituted for asparagine at a position corresponding to amino acid 429 of SEQ ID NO: 1;
(vii) methionine substituted for valine at a position corresponding to amino acid 462 of SEQ ID NO: 1;
(viii) glycine substituted for glutamic acid at a position corresponding to amino acid 492 of SEQ ID NO: 1;
(ix) valine substituted for glutamic acid at a position corresponding to amino acid 501 of SEQ ID NO: 1;
(x) glutamic acid substituted lysine at a position corresponding to amino acid 545 of SEQ ID NO: 1;
(xi) alanine substituted for valine at a position corresponding to amino acid 547 of SEQ ID NO: 1;
(xii) glutamine substituted for histidine at a position corresponding to amino acid 464 of SEQ ID NO: 1;
(xiii) tryptophan substituted for threonine at a position corresponding to amino acid 422 of SEQ ID NO: 1;
(xiv) methionine substituted for threonine at a position corresponding to amino acid 422 of SEQ ID NO: 1;
(xv) glutamine substituted for histidine at a position corresponding to amino acid 510 of SEQ ID NO: 1;
(xvi) arginine substituted for histidine at a position corresponding to amino acid 510 of SEQ ID NO: 1;
(xvii) phenylalanine substituted for histidine at a position corresponding to amino acid 535 of SEQ ID NO: 1;
(xviii) glutamine substituted for histidine at a position corresponding to amino acid 535 of SEQ ID NO: 1;
(xix) tryptophan substituted for leucine at a position corresponding to amino acid 463 of SEQ ID NO: 1;
(xx) methionine substituted for threonine at a position corresponding to amino acid 467 of SEQ ID NO: 1;
(xxi) lysine substituted for glutamic acid at a position corresponding to amino acid 505 of SEQ ID NO: 1;
(xxii) glycine substituted for glutamic acid at a position corresponding to amino acid 505 of SEQ ID NO: 1;
(xxiii) arginine substituted for glutamic acid at a position corresponding to amino acid 505 of SEQ ID NO: 1;
(xxiv) tryptophan substituted for phenylalanine at a position corresponding to amino acid 509 of SEQ ID NO: 1;
(xxv) glutamic acid substituted for lysine at a position corresponding to amino acid 519 of SEQ ID NO: 1; and
(xxvi) combinations thereof.

In one example, the albumin variant or fragment thereof comprises:
(i) an amino acid substitution at a position corresponding to amino acid 464 of SEQ ID NO: 1; or
(ii) an amino acid substitution at a position corresponding to amino acid 535 of SEQ ID NO: 1; or
(iii) an amino acid substitution at a position corresponding to amino acid 422 of SEQ ID NO: 1, and an amino acid substitution at a position corresponding to amino acid 464 of SEQ ID NO: 1; or
(iv) an amino acid substitution at a position corresponding to amino acid 464 of SEQ ID NO: 1 and an amino acid substitution at a position corresponding to amino acid 535 of SEQ ID NO: 1; or
(v) an amino acid substitution at a position corresponding to amino acid 464 of SEQ ID NO: 1 and an amino acid substitution at a position corresponding to amino acid 509 of SEQ ID NO: 1; or
(vi) an amino acid substitution at a position corresponding to amino acid 464 of SEQ ID NO: 1 and an amino acid substitution at a position corresponding to amino acid 519 of SEQ ID NO: 1; or
(vii) an amino acid substitution at a position corresponding to amino acid 464 of SEQ ID NO: 1 and an amino acid substitution at a position corresponding to amino acid 510 of SEQ ID NO: 1; or
(viii) an amino acid substitution at a position corresponding to amino acid 464 of SEQ ID NO: 1 and an amino acid substitution at a position corresponding to amino acid 505 of SEQ ID NO: 1; or
(ix) an amino acid substitution at a position corresponding to amino acid 464 of SEQ ID NO: 1 and an amino acid substitution at a position corresponding to amino acid 467 of SEQ ID NO: 1;
(x) an amino acid substitution at a position corresponding to amino acid 418 of SEQ ID NO: 1, an amino acid substitution at a position corresponding to amino acid 420 of SEQ ID NO: 1, an amino acid substitution at a position corresponding to amino acid 505 of SEQ ID NO: 1 and an amino acid substitution at a position corresponding to amino acid 547 of SEQ ID NO: 1; or
(xi) an amino acid substitution at a position corresponding to amino acid 464 of SEQ ID NO: 1 and an amino acid substitution at a position corresponding to amino acid 463 of SEQ ID NO: 1.

An exemplary albumin variant or fragment thereof comprises one or more amino acid substitutions selected from the group consisting of:
(i) glutamine substituted for histidine at a position corresponding to amino acid 464 of SEQ ID NO: 1;
(ii) tryptophan substituted for threonine at a position corresponding to amino acid 422 of SEQ ID NO: 1;
(iii) glutamine substituted for histidine at a position corresponding to amino acid 510 of SEQ ID NO: 1;
(iv) phenylalanine substituted for histidine at a position corresponding to amino acid 535 of SEQ ID NO: 1; and
(v) combinations thereof.

In one example, an exemplary albumin variant or fragment thereof comprises a glutamine substituted for histidine at a position corresponding to amino acid 464 of SEQ ID NO: 1.

In one example, the albumin variant or fragment thereof comprises:
(i) an amino acid substitution at a position corresponding to amino acid 464 of SEQ ID NO: 1; or
(ii) an amino acid substitution at a position corresponding to amino acid 535 of SEQ ID NO: 1; or
(iii) an amino acid substitution at a position corresponding to amino acid 422 of SEQ ID NO: 1, and an amino acid substitution at a position corresponding to amino acid 464 of SEQ ID NO: 1; or
(iv) an amino acid substitution at a position corresponding to amino acid 464 of SEQ ID NO: 1 and an amino acid substitution at a position corresponding to amino acid 535 of SEQ ID NO: 1.

In one example, the albumin variant or fragment thereof comprises:
(i) glutamine substituted for histidine at a position corresponding to amino acid 464 of SEQ ID NO: 1; or
(ii) phenylalanine substituted for histidine at a position corresponding to amino acid 535 of SEQ ID NO: 1; or
(iii) glutamine substituted for histidine at a position corresponding to amino acid 535 of SEQ ID NO: 1; or
(iv) tryptophan substituted for threonine at a position corresponding to amino acid 422 of SEQ ID NO: 1, and glutamine substituted for histidine at a position corresponding to amino acid 464 of SEQ ID NO: 1; or
(v) glutamine substituted for histidine at a position corresponding to amino acid 464 of SEQ ID NO: 1 and phenylalanine substituted for histidine at a position corresponding to amino acid 535 of SEQ ID NO: 1; or
(vi) methionine substituted for threonine at a position corresponding to amino acid 422 of SEQ ID NO: 1, and glutamine substituted for histidine at a position corresponding to amino acid 464 of SEQ ID NO: 1; or
(vii) tyrosine substituted for phenylalanine at a position corresponding to amino acid 509 of SEQ ID NO: 1, and glutamine substituted for histidine at a position corresponding to amino acid 464 of SEQ ID NO: 1; or
(viii) glutamic acid substituted for lysine at a position corresponding to amino acid 519 of SEQ ID NO: 1, and glutamine substituted for histidine at a position corresponding to amino acid 464 of SEQ ID NO: 1; or
(ix) arginine substituted for histidine at a position corresponding to amino acid 510 of SEQ ID NO: 1, and glutamine substituted for histidine at a position corresponding to amino acid 464 of SEQ ID NO: 1; or
(x) arginine substituted for histidine at a position corresponding to amino acid 510 of SEQ ID NO: 1, and phenylalanine substituted for histidine at a position corresponding to amino acid 535 of SEQ ID NO: 1; or
(xi) lysine substituted for glutamic acid at a position corresponding to amino acid 505 of SEQ ID NO: 1, and glutamine substituted for histidine at a position corresponding to amino acid 464 of SEQ ID NO: 1; or
(xii) methionine substituted for threonine at a position corresponding to amino acid 467 of SEQ ID NO: 1, and glutamine substituted for histidine at a position corresponding to amino acid 464 of SEQ ID NO: 1; or
(xiii) tyrosine substituted for leucine at a position corresponding to amino acid 519 of SEQ ID NO: 1, and glutamine substituted for histidine at a position corresponding to amino acid 464 of SEQ ID NO: 1.

In one example, the albumin variant or fragment thereof comprises:
(i) glutamine substituted for histidine at a position corresponding to amino acid 464 of SEQ ID NO: 1; or
(ii) phenylalanine substituted for histidine at a position corresponding to amino acid 535 of SEQ ID NO: 1; or
(iii) glutamine substituted for histidine at a position corresponding to amino acid 535 of SEQ ID NO: 1; or
(iv) tryptophan substituted for threonine at a position corresponding to amino acid 422 of SEQ ID NO: 1, and glutamine substituted for histidine at a position corresponding to amino acid 464 of SEQ ID NO: 1; or
(v) glutamine substituted for histidine at a position corresponding to amino acid 464 of SEQ ID NO: 1 and phenylalanine substituted for histidine at a position corresponding to amino acid 535 of SEQ ID NO: 1.

In an example of the disclosure, the immunoglobulin Fc domain or fragment thereof is a Fc domain from an IgG or fragment thereof.

In an example of the disclosure, the immunoglobulin Fc domain or fragment thereof is a Fc domain from an IgG1 or fragment thereof.

In an example of the disclosure, the immunoglobulin Fc domain or fragment thereof is a Fc domain from an IgG4 or fragment thereof. In an example of the disclosure, the immunoglobulin Fc domain or fragment thereof is not from an IgG4 or fragment thereof.

In an example of the disclosure, the immunoglobulin Fc domain or fragment thereof is an Fc domain variant or fragment thereof. For example, the Fc domain variant or fragment thereof is an IgG1 Fc domain variant or fragment thereof or IgG4 Fc domain variant or fragment thereof.

In one example, the IgG1 Fc domain variant or fragment thereof or the IgG1 Fc domain variant or fragment thereof binds with increased affinity to human FcRn compared to an IgG1 Fc domain set forth in SEQ ID NO: 2.

For example, the binding affinity is measured at neutral and/or acidic pH. Exemplary K_{D}s of a Fc domain variant or fragment thereof are described above in relation to the K_{D} for an antagonist of the disclosure for FcRn and are to be taken to apply mutatis mutandis to the present examples of the disclosure.

In one example, the Fc domain variant or fragment thereof comprises one or more amino acid substitutions selected from the group consisting of:
(i) tyrosine substituted for methionine at a position corresponding to amino acid 252 according to the EU numbering system;
(ii) threonine substituted for serine at a position corresponding to amino acid 254 according to the EU numbering system;
(iii) glutamic acid substituted for threonine at a position corresponding to amino acid 256 according to the EU numbering system;
(iv) glutamic acid substituted for asparagine at a position corresponding to amino acid 286 according to the EU numbering system;
(v) proline substituted for valine at a position corresponding to amino acid 308 according to the EU numbering system;
(vi) lysine substituted for histidine at a position corresponding to amino acid 433 according to the EU numbering system;
(vii) tyrosine substituted for asparagine at a position corresponding to amino acid 434 according to the EU numbering system;
(viii) phenylalanine substituted for asparagine at a position corresponding to amino acid 434 according to the EU numbering system;
(ix) histidine substituted for tyrosine at a position corresponding to amino acid 436 according to the EU numbering system; and
(x) combinations thereof.

For example, the Fc domain variant or fragment thereof comprises:
(i) tyrosine substituted for methionine at a position corresponding to amino acid 252 according to the EU numbering system, proline substituted for valine at a position corresponding to amino acid 308 according to the EU numbering system and tyrosine substituted for asparagine at a position corresponding to amino acid 434 according to the EU numbering system; or
(ii) tyrosine substituted for methionine at a position corresponding to amino acid 252 according to the EU numbering system, threonine substituted for serine at a position corresponding to amino acid 254 according to the EU numbering system and glutamic acid substituted for threonine at a position corresponding to amino acid 256 according to the EU numbering system; or
(iii) tyrosine substituted for methionine at a position corresponding to amino acid 252 according to the EU numbering system, glutamic acid substituted for asparagine at a position corresponding to amino acid 286 according to the EU numbering system and tyrosine substituted for asparagine at a position corresponding to amino acid 434 according to the EU numbering system; or
(iv) tyrosine substituted for methionine at a position corresponding to amino acid 252 according to the EU numbering system, threonine substituted for serine at a position corresponding to amino acid 254 according to the EU numbering system, glutamic acid substituted for threonine at a position corresponding to amino acid 256 according to the EU numbering system, lysine substituted for histidine at a position corresponding to amino acid 433 according to the EU numbering system and phenylalanine substituted for asparagine at a position corresponding to amino acid 434 according to the EU numbering system.

In one example, a FcRn antagonist of the disclosure comprises:
(i) the Fc domain comprises tyrosine substituted for methionine at a position corresponding to amino acid 252 according to the EU numbering system, proline substituted for valine at a position corresponding to amino acid 308 according to the EU numbering system and tyrosine substituted for asparagine at a position corresponding to amino acid 434 according to the EU numbering system; and
(ii) the albumin comprises glutamine substituted for histidine at a position corresponding to amino acid 464 according to the EU numbering system.

In one example, the Fc domain or fragment thereof comprises tyrosine substituted for methionine at a position corresponding to amino acid 252 according to the EU numbering system, threonine substituted for serine at a position corresponding to amino acid 254 according to the EU numbering system, glutamic acid substituted for threonine at a position corresponding to amino acid 256 according to the EU numbering system, lysine substituted for histidine at a position corresponding to amino acid 433 according to the EU numbering system and phenylalanine substituted for asparagine at a position corresponding to amino acid 434 according to the EU numbering system.

In one example, the Fc domain or fragment thereof comprises tyrosine substituted for methionine at a position corresponding to amino acid 252 according to the EU numbering system, proline substituted for valine at a position corresponding to amino acid 308 according to the EU numbering system and tyrosine substituted for asparagine at a position corresponding to amino acid 434 according to the EU numbering system.

In one example, the Fc domain or fragment thereof is linked to one albumin. In some examples, a Fc domain can form a dimer of two Fc domains, an antagonist of the disclosure can comprise a Fc domain dimer, wherein each Fc domain is linked to an albumin, i.e., the antagonist comprises two albumins.

In another example, one of the Fc domains in the Fc domain dimer is linked to an albumin or fragment thereof and the other Fc domain is not linked to an albumin or fragment thereof.

In one example, each Fc domain of the FcRn antagonist is linked to two or more albumins or fragments thereof.

In one example, each Fc domain is linked to one albumin or fragment thereof.

In one example, the Fc domain or fragment thereof is indirectly linked to the albumin or fragment thereof, e.g., via a linker. For example, the antagonist comprises a linker positioned between the Fc domain or fragment thereof and the albumin or fragment thereof. In one example, the linker is a peptide or polypeptide. For example, the linker is a peptide linker comprising between 2 and 31 amino acids in length. For example, the linker comprises glycine or glycine and serine. For example, the linker comprises one or more repeats of Gly₄Ser.

In another example, the Fc domain or fragment thereof is directly linked to an albumin or fragment thereof, e.g., without an intervening linker.

In an exemplary form of the disclosure, the C-terminus of the Fc domain or fragment thereof is indirectly or directly linked to the N-terminus of an albumin or fragment thereof.

In another form of the disclosure, the C-terminus of the albumin or fragment thereof is indirectly or directly linked to the N-terminus of the Fc domain or fragment thereof.

In another form of the disclosure, the C-terminus of the Fc domain or fragment thereof is indirectly or directly linked to the C-terminus of an albumin or fragment thereof.

In another form of the disclosure, the N-terminus of the Fc domain or fragment thereof is indirectly or directly linked to the N-terminus of an albumin or fragment thereof.

In one example, the Fc domain or fragment thereof is a monomeric Fc domain or fragment thereof. In another example, the albumin or fragment thereof is a monomeric albumin or fragment thereof. For example, the monomeric Fc domain is fused to the monomeric albumin.

In another example, the FcRn antagonist comprises a single albumin or fragment thereof. In such examples, the FcRn antagonist is a heterodimer. For example, the heterodimeric FcRn antagonist comprises a single albumin or fragment thereof fused to a dimeric Fc domain or fragment thereof.

In one example, the FcRn antagonist comprises two albumins or fragments thereof. For example, the FcRn antagonist is a homodimer.

The present disclosure additionally provides a FcRn antagonist comprising:
(i) an immunoglobulin Fc domain or fragment thereof comprising tyrosine substituted for methionine at a position corresponding to amino acid 252 according to the EU numbering system, threonine substituted for serine at a position corresponding to amino acid 254 according to the EU numbering system, glutamic acid substituted for threonine at a position corresponding to amino acid 256 according to the EU numbering system, lysine substituted for histidine at a position corresponding to amino acid 433 according to the EU numbering system and phenylalanine substituted for asparagine at a position corresponding to amino acid 434 according to the EU numbering system; and
(ii) an albumin or fragment thereof comprising glutamine substituted for histidine at a position corresponding to amino acid 464 of SEQ ID NO: 1,
   wherein the C-terminus of the Fc domain or fragment thereof is indirectly or directly linked to the N-terminus of the albumin or fragment thereof.

The present disclosure additionally provides a FcRn antagonist comprising a sequence set forth in SEQ ID NO: 8.

The present disclosure additionally provides a FcRn antagonist comprising:
(i) an immunoglobulin Fc domain or fragment thereof comprising tyrosine substituted for methionine at a position corresponding to amino acid 252 according to the EU numbering system, proline substituted for valine at a position corresponding to amino acid 308 according to the EU numbering system and tyrosine substituted for asparagine at a position corresponding to amino acid 434 according to the EU numbering system; and
(ii) an albumin or fragment thereof comprising glutamine substituted for histidine at a position corresponding to amino acid 464 of SEQ ID NO: 1,
   wherein the C-terminus of the Fc domain or fragment thereof is indirectly or directly linked to the N-terminus of the albumin or fragment thereof.

The present disclosure additionally provides a FcRn antagonist comprising a sequence set forth in SEQ ID NO: 11.

FcRn antagonists of the disclosure comprise a sequence set forth in any one of SEQ ID NOs: 3-12. For example, a FcRn antagonist of the disclosure comprise a sequence set forth in any one of SEQ ID NOs: 3, 5, 8, 9 or 11.

In one example, the FcRn antagonist comprises a sequence set forth in SEQ ID NO: 3. In one example, the FcRn antagonist comprises a sequence set forth in SEQ ID NO: 5. In one example, the FcRn antagonist comprises a sequence set forth in SEQ ID NO: 8. In one example, the FcRn antagonist comprises a sequence set forth in SEQ ID NO: 9. In one example, the FcRn antagonist comprises a sequence set forth in SEQ ID NO: 11.

The present disclosure additionally provides a composition comprising the FcRn antagonist of the disclosure and a pharmaceutically acceptable carrier.

The disclosure additionally provides a nucleic acid encoding the FcRn antagonist of the disclosure. In one example, the nucleic acid is DNA. In another example, the nucleic acid is RNA, including modified forms thereof. In one example, the nucleic acid is linked to nucleic acid required for expression of the FcRn antagonist. In some examples, the nucleic acid is contained within a liposome or particle, e.g., lipid nanoparticle (LNP). In some examples, the nucleic acid, liposome or particle is within a pharmaceutical formulation.

The present disclosure additionally provides a FcRn antagonist or nucleic acid or composition as disclosed herein for use in inhibiting immunoglobulin binding to FcRn and/or reducing circulating levels of immunoglobulin in a subject. The disclosure additionally provides for use of a FcRn antagonist or nucleic acid or composition as disclosed herein in the manufacture of a medicament for inhibiting immunoglobulin binding to FcRn and/or reducing circulating levels of immunoglobulin in a subject. The disclosure additionally provides a method of inhibiting immunoglobulin binding to FcRn and/or reducing circulating levels of immunoglobulin in a subject, the method comprising administering the FcRn antagonist or nucleic acid or composition as disclosed herein.

The disclosure also provides a FcRn antagonist or nucleic acid or composition of the disclosure for use in reducing circulating Fc-containing proteins and/or antibodies in a subject, e.g., a subject suffering from an autoimmune disease and having pathogenic autoantibodies or a subject that has developed anti-drug antibodies and/or is at risk of developing anti-drug antibodies. In one example, the time over which the level of antibodies in the subject is reduced is longer compared to the time over which the level of antibodies is reduced in the subject if administered the Fc domain or fragment thereof from the FcRn antagonist alone, i.e., in the absence of the albumin or fragment thereof.

The disclosure also provides a FcRn antagonist or nucleic acid or composition of the disclosure for use in treating or preventing progression of an antibody-mediated disorder in a subject in need thereof.

The disclosure additionally provides a method of reducing circulating autoantibodies in a subject, the method comprising administering the FcRn antagonist or nucleic acid or composition of the disclosure to the subject. In one example, the time over which the level of autoantibodies in the subject is reduced is longer compared to the time over which the level of autoantibodies is reduced in the subject if administered the Fc domain or fragment thereof from the FcRn antagonist alone, i.e., in the absence of the albumin or fragment thereof.

The disclosure additionally provides a method of reducing circulating anti-drug antibodies in a subject, the method comprising administering the FcRn antagonist or nucleic acid or composition of the disclosure to the subject. In one example, the time over which the level of anti-drug antibodies in the subject is reduced is longer compared to the time over which the level of anti-drug antibodies is reduced in the subject if administered the Fc domain or fragment thereof from the FcRn antagonist alone, i.e., in the absence of the albumin or fragment thereof.

The disclosure also provides a method of treating or preventing progression of an antibody-mediated disorder in a subject in need thereof, the method comprising administering the FcRn antagonist or nucleic acid or composition of the disclosure to the subject.

The disclosure also provides a use of the FcRn antagonist or nucleic acid or composition of the disclosure in the manufacture of a medicament for reducing circulating autoantibodies in a subject.

The disclosure also provides a use of the FcRn antagonist or nucleic acid or composition of the disclosure in the manufacture of a medicament for reducing circulating anti-drug antibodies in a subject.

The disclosure also provides a use of the FcRn antagonist or nucleic acid or composition of the disclosure in the manufacture of a medicament for treating or preventing progression of an antibody-mediated disorder in a subject.

In one example, the subject is suffering from an autoimmune disease, has developed undesirable antibodies and/or is at risk of developing undesirable antibodies. For example, the undesirable antibodies are autoantibodies. In another example, the undesirable antibodies are anti-drug antibodies.

In one example, the subject has developed or is at risk of developing anti-drug antibodies. For example, the subject is receiving treatment therapy with a protein therapy, e.g., an antibody or an immunoadhesin or a clotting factor and has developed or is at risk of developing antibodies against the therapy.

In one example, the FcRn antagonist is administered in an amount effective to:
(i) reduce endogenous IgG levels by at least 1× or 2× or 3× or 4× or 5× (compared to in the absence of FcRn antagonist administration); and/or
(ii) reduce endogenous albumin levels no more than 20% or 15% or 10% or 5% or 1% (compared to in the absence of FcRn antagonist administration).

In one example, the FcRn antagonist is administered in an amount effective to reduce endogenous IgG levels by at least about 10% or about 20%, or about 30%, or about 40%, or about 50%, or about 60%, or about 70%, or about 80%, or about 90% compared to the level observed following administration of a FcRn antagonist comprising wild-type human albumin and/or administration of a Fc domain or fragment thereof from the FcRn antagonist alone. For example, the FcRn antagonist is administered in an amount effective to reduce endogenous IgG levels by at least about 40% to about 80% compared to the level observed following administration of a FcRn antagonist comprising wild-type human albumin and/or administration of a Fc domain or fragment thereof from the FcRn antagonist alone. In one example, the FcRn antagonist is administered in an amount effective to reduce endogenous IgG levels by at least about 10% compared to the level observed following administration of a FcRn antagonist comprising wild-type human albumin and/or administration of a Fc domain or fragment thereof from the FcRn antagonist alone. In one example, the FcRn antagonist is administered in an amount effective to reduce endogenous IgG levels by at least about 20% compared to the level observed following administration of a FcRn antagonist comprising wild-type human albumin and/or administration of a Fc domain or fragment thereof from the FcRn antagonist alone. In one example, the FcRn antagonist is administered in an amount effective to reduce endogenous IgG levels by at least about 30% compared to the level observed following administration of a FcRn antagonist comprising wild-type human albumin and/or administration of a Fc domain or fragment thereof from the FcRn antagonist alone. In one example, the FcRn antagonist is administered in an amount effective to reduce endogenous IgG levels by at least about 40% compared to the level observed following administration of a FcRn antagonist comprising wild-type human albumin and/or administration of a Fc domain or fragment thereof from the FcRn antagonist alone. In one example, the FcRn antagonist is administered in an amount effective to reduce endogenous IgG levels by at least about 50% compared to the level observed following administration of a FcRn antagonist comprising wild-type human albumin and/or administration of a Fc domain or fragment thereof from the FcRn antagonist alone. In one example, the FcRn antagonist is administered in an amount effective to reduce endogenous IgG levels by at least about 60% compared to the level observed following administration of a FcRn antagonist comprising wild-type human albumin and/or administration of a Fc domain or fragment thereof from the FcRn antagonist alone. In one example, the FcRn antagonist is administered in an amount effective to reduce endogenous IgG levels by at least about 70% compared to the level observed following administration of a FcRn antagonist comprising wild-type human albumin and/or administration of a Fc domain or fragment thereof from the FcRn antagonist alone. In one example, the FcRn antagonist is administered in an amount effective to reduce endogenous IgG levels by at least about 80% compared to the level observed following administration of a FcRn antagonist comprising wild-type human albumin and/or administration of a Fc domain or fragment thereof from the FcRn antagonist alone. In one example, the FcRn antagonist is administered in an amount effective to reduce endogenous IgG levels by at least about 90% compared to the level observed following administration of a FcRn antagonist comprising wild-type human albumin and/or administration of a Fc domain or fragment thereof from the FcRn antagonist alone.

In one example, the FcRn antagonist antagonizes IgG recycling, however, does not significantly, substantially or detectably reduce endogenous albumin levels.

In one example, the FcRn antagonist antagonizes IgG recycling, however does not substantially antagonize albumin recycling. As discussed above, the inventors consider this an advantage of FcRn antagonists comprising an albumin or fragment thereof that does not substantially bind to FcRn, since it does not interfere with endogenous albumin recycling and/or homeostasis.

In one example, administration of the FcRn antagonist does not induce dyslipidemia and/or induces a smaller increase in serum cholesterol levels compared to the level observed following administration of a FcRn antagonist comprising wild-type human albumin. For example, administration of the FcRn antagonist does not induce dyslipidemia. In another example, administration of the FcRn antagonist induces a smaller increase in serum cholesterol levels compared to the level observed following administration of a FcRn antagonist comprising wild-type human albumin.

In one example, administration of the FcRn antagonist does not change the level of endogenous albumin, cholesterol, low-density lipoprotein (LDL) cholesterol and/or triglycerides and/or induces a smaller change compared to the level observed following administration of a FcRn antagonist comprising wild-type human albumin. In one example, administration of the FcRn antagonist does not change the level of endogenous albumin and/or induces a smaller change compared to the level observed following administration of a FcRn antagonist comprising wild-type human albumin. In one example, administration of the FcRn antagonist does not change the level of endogenous cholesterol and/or induces a smaller change compared to the level observed following administration of a FcRn antagonist comprising wild-type human albumin. In one example, administration of the FcRn antagonist does not change the level of endogenous low-density lipoprotein (LDL) cholesterol and/or induces a smaller change compared to the level observed following administration of a FcRn antagonist comprising wild-type human albumin. In one example, administration of the FcRn antagonist does not change the level of endogenous triglycerides and/or induces a smaller change compared to the level observed following administration of a FcRn antagonist comprising wild-type human albumin.

In one example, the FcRn antagonist reduces endogenous IgG levels within at least 2 days, 3 days, 4, days, 5 days, 6 days, 7 days, 8 days, 9 days, 10 days, 11 days, 12 days, 13 days or 14 days of administration. For example, the FcRn antagonist reduces endogenous IgG levels within 2 to 9 days of administration. In one example, the FcRn antagonist reduces endogenous IgG levels within at least 2 days of administration. In one example, the FcRn antagonist reduces endogenous IgG levels within at least 3 days of administration. In one example, the FcRn antagonist reduces endogenous IgG levels within at least 4 days of administration. In one example, the FcRn antagonist reduces endogenous IgG levels within at least 5 days of administration. In one example, the FcRn antagonist reduces endogenous IgG levels within at least 6 days of administration. In one example, the FcRn antagonist reduces endogenous IgG levels within at least 7 days of administration. In one example, the FcRn antagonist reduces endogenous IgG levels within at least 8 days of administration. In one example, the FcRn antagonist reduces endogenous IgG levels within at least 9 days of administration. In one example, the FcRn antagonist reduces endogenous IgG levels within at least 10 days of administration. In one example, the FcRn antagonist reduces endogenous IgG levels within at least 11 days of administration. In one example, the FcRn antagonist reduces endogenous IgG levels within at least 12 days of administration. In one example, the FcRn antagonist reduces endogenous IgG levels within at least 13 days of administration. In one example, the FcRn antagonist reduces endogenous IgG levels within at least 14 days of administration.

In one example, administration of the FcRn antagonist reduces endogenous IgG levels by at least about 10% or about 20%, or about 30%, or about 40%, or about 50%, or about 60%, or about 70%, or about 80%, or about 90%. For example, administration of the FcRn antagonist reduces endogenous IgG levels by at least about 50% to about 80%. In one example, administration of the FcRn antagonist reduces endogenous IgG levels by at least about 10% to about 90%. In one example, administration of the FcRn antagonist reduces endogenous IgG levels by at least about 10%. In one example, administration of the FcRn antagonist reduces endogenous IgG levels by at least about 20%. In one example, administration of the FcRn antagonist reduces endogenous IgG levels by at least about 30%. In one example, administration of the FcRn antagonist reduces endogenous IgG levels by at least about 40%. In one example, administration of the FcRn antagonist reduces endogenous IgG levels by at least about 50%. In one example, administration of the FcRn antagonist reduces endogenous IgG levels by at least about 60%. In one example, administration of the FcRn antagonist reduces endogenous IgG levels by at least about 70%. In one example, administration of the FcRn antagonist reduces endogenous IgG levels by at least about 80%. In one example, administration of the FcRn antagonist reduces endogenous IgG levels by at least about 90%.

In one example, administration of the FcRn antagonist does not change or substantially change endogenous IgA levels.

In one example, the FcRn antagonist increases endogenous IgM levels within at least 5 days, 6 days, 7 days, 8 days or 9 days of administration. In one example, the FcRn antagonist increases endogenous IgM levels within at least 5 days of administration. In one example, the FcRn antagonist increases endogenous IgM levels within at least 6 days of administration. In one example, the FcRn antagonist increases endogenous IgM levels within at least 7 days of administration. In one example, the FcRn antagonist increases endogenous IgM levels within at least 8 days of administration. In one example, the FcRn antagonist increases endogenous IgM levels within at least 9 days of administration.

In one example, administration of the FcRn antagonist reduces the volume of distribution (V_{SS}) compared to the V_{SS} observed following administration of a FcRn antagonist comprising wild-type human albumin and/or administration of a Fc domain or fragment thereof from the FcRn antagonist alone (e.g., huG1FcYTEKF). In one example, administration of the FcRn antagonist reduces the V_{SS} by a least about 100 mL, 150 mL, 200 mL, 250 mL, 300 mL, 350 mL, 400 mL, 450 mL or 500 mL compared to the V_{SS} observed following administration of a Fc domain or fragment thereof from the FcRn antagonist alone. For example, administration of the FcRn antagonist reduces the V_{SS} by a least about 400 mL to 500 mL compared to the V_{SS} observed following administration of a Fc domain or fragment thereof from the FcRn antagonist alone. In one example, administration of the FcRn antagonist reduces the V_{SS} by a least about 100 mL compared to the V_{SS} observed following administration of a Fc domain or fragment thereof from the FcRn antagonist alone. In one example, administration of the FcRn antagonist reduces the V_{SS} by a least about 200 mL compared to the V_{SS} observed following administration of a Fc domain or fragment thereof from the FcRn antagonist alone. In one example, administration of the FcRn antagonist reduces the V_{SS} by a least about 250 mL compared to the V_{SS} observed following administration of a Fc domain or fragment thereof from the FcRn antagonist alone. In one example, administration of the FcRn antagonist reduces the V_{SS} by a least about 300 mL compared to the V_{SS} observed following administration of a Fc domain or fragment thereof from the FcRn antagonist alone. In one example, administration of the FcRn antagonist reduces the V_{SS} by a least about 2500 mL compared to the V_{SS} observed following administration of a Fc domain or fragment thereof from the FcRn antagonist alone. In one example, administration of the FcRn antagonist reduces the V_{SS} by a least about 400 mL compared to the V_{SS} observed following administration of a Fc domain or fragment thereof from the FcRn antagonist alone. In one example, administration of the FcRn antagonist reduces the V_{SS} by a least about 450 mL compared to the V_{SS} observed following administration of a Fc domain or fragment thereof from the FcRn antagonist alone. In one example, administration of the FcRn antagonist reduces the V_{SS} by a least about 500 mL compared to the V_{SS} observed following administration of a Fc domain or fragment thereof from the FcRn antagonist alone.

In one example, administration of the FcRn antagonist reduces the V_{SS} by a least about 40 mL, 50 mL, 60 mL, 70 mL or 80 mL compared to the V_{SS} observed following administration of a FcRn antagonist comprising wild-type human albumin. For example, administration of the FcRn antagonist reduces the V_{SS} by a least about 40 mL to 80 mL compared to the V_{SS} observed following administration of a FcRn antagonist comprising wild-type human albumin. In one example, administration of the FcRn antagonist reduces the V_{SS} by a least about 40 mL compared to the V_{SS} observed following administration of a FcRn antagonist comprising wild-type human albumin. In one example, administration of the FcRn antagonist reduces the V_{SS} by a least about 50 mL compared to the V_{SS} observed following administration of a FcRn antagonist comprising wild-type human albumin. In one example, administration of the FcRn antagonist reduces the V_{SS} by a least about 60 mL compared to the V_{SS} observed following administration of a FcRn antagonist comprising wild-type human albumin. In one example, administration of the FcRn antagonist reduces the V_{SS} by a least about 70 mL compared to the V_{SS} observed following administration of a FcRn antagonist comprising wild-type human albumin. In one example, administration of the FcRn antagonist reduces the V_{SS} by a least about 80 mL compared to the V_{SS} observed following administration of a FcRn antagonist comprising wild-type human albumin.

In one example, administration of the FcRn antagonist increases the area under the curve (AUC) compared to the AUC observed following administration of a Fc domain or fragment thereof from the FcRn antagonist alone. For example, administration of the FcRn antagonist increases the AUC by at least 250 μg/mL*h/mg, 300 μg/mL*h/mg, 350 μg/mL*h/mg, 400 μg/mL*h/mg, 450 μg/mL*h/mg, or 500 μg/mL*h/mg compared to the AUC observed following administration of a Fc domain or fragment thereof from the FcRn antagonist alone. In one example, administration of the FcRn antagonist increases the AUC by at least 250 μg/mL*h/mg to 500 μg/mL*h/mg compared to the AUC observed following administration of a Fc domain or fragment thereof from the FcRn antagonist alone. In one example, administration of the FcRn antagonist increases the AUC by at least 250 μg/mL*h/mg compared to the AUC observed following administration of a Fc domain or fragment thereof from the FcRn antagonist alone. In one example, administration of the FcRn antagonist increases the AUC by at least 300 μg/mL*h/mg compared to the AUC observed following administration of a Fc domain or fragment thereof from the FcRn antagonist alone. In one example, administration of the FcRn antagonist increases the AUC by at least =350 μg/mL*h/mg compared to the AUC observed following administration of a Fc domain or fragment thereof from the FcRn antagonist alone. In one example, administration of the FcRn antagonist increases the AUC by at least 400 μg/mL*h/mg compared to the AUC observed following administration of a Fc domain or fragment thereof from the FcRn antagonist alone. In one example, administration of the FcRn antagonist increases the AUC by at least 450 μg/mL*h/mg compared to the AUC observed following administration of a Fc domain or fragment thereof from the FcRn antagonist alone. In one example, administration of the FcRn antagonist increases the AUC by at least 500 μg/mL*h/mg compared to the AUC observed following administration of a Fc domain or fragment thereof from the FcRn antagonist alone.

In one example, administration of the FcRn antagonist increases the AUC compared to the AUC observed following administration of a FcRn antagonist comprising wild-type human albumin. For example, administration of the FcRn antagonist increases the AUC by at least 100 μg/mL*h/mg, 150 μg/mL*h/mg, 200 μg/mL*h/mg or 250 μg/mL*h/mg compared to the AUC observed following administration of a FcRn antagonist comprising wild-type human albumin. In one example, administration of the FcRn antagonist increases the AUC by at least 100 μg/mL*h/mg to 250 μg/mL*h/mg compared to the AUC observed following administration of a FcRn antagonist comprising wild-type human albumin. In one example, administration of the FcRn antagonist increases the AUC by at least 100 μg/mL*h/mg compared to the AUC observed following administration of a FcRn antagonist comprising wild-type human albumin. In one example, administration of the FcRn antagonist increases the AUC by at least 150 μg/mL*h/mg compared to the AUC observed following administration of a FcRn antagonist comprising wild-type human albumin. In one example, administration of the FcRn antagonist increases the AUC by at least 200 μg/mL*h/mg compared to the AUC observed following administration of a FcRn antagonist comprising wild-type human albumin. In one example, administration of the FcRn antagonist increases the AUC by at least 250 μg/mL*h/mg compared to the AUC observed following administration of a FcRn antagonist comprising wild-type human albumin.

In another example, administration of the FcRn antagonist cross-links cell surface FcRn at a lower level than a FcRn antagonist comprising wild-type human albumin.

In one example, the subject has received, is receiving, or will receive an additional therapy. For example, the additional therapy is a steroidal immune modulator, plasmapheresis and/or IVIg therapy.

The disclosure additionally provides a kit for use in reducing circulating autoantibodies in a subject in need thereof, the kit comprising:
(i) at least one FcRn antagonist or composition of the disclosure;
(ii) instructions for using the kit in reducing circulating autoantibodies in the subject; and
(iii) optionally, at least one additional therapy.

The disclosure also provides a kit for treating or preventing progression of an antibody-mediated disorder in a subject in need thereof, the kit comprising:
(i) at least one FcRn antagonist or composition of the disclosure;
(ii) instructions for using the kit in treating or preventing progression of an antibody-mediated disorder in the subject; and
(iii) optionally, at least one additional therapy.

### BRIEF DESCRIPTION OF THE DRAWINGS

FIG. 1A is a graphical representation showing levels of an IgG ‘tracer’ antibody in human FcRn (huFcRn) transgenic mice dosed with various FcRn antagonists of the disclosure.

FIG. 1B is a graphical representation showing levels of an IgG ‘tracer’ antibody in human FcRn (huFcRn) transgenic mice dosed with various FcRn antagonists of the disclosure.

FIG. 1C is a graphical representation showing in vivo half-life of various FcRn antagonists of the disclosure following administration to huFcRn transgenic mice.

FIGS. 2A and B are graphical representations showing levels of tracer antibody (CSL360) in huFcRn transgenic mice dosed with various FcRn antagonists of the disclosure.

FIG. 2C is a graphical representation showing the pharmacokinetics of FcRn antagonists as described herein when administered intravenously or subcutaneously. The antagonists are as indicated and described herein. As shown, the antagonists show comparable pharmacokinetics whether administered intravenously or subcutaneously.

FIG. 2D is a graphical representation showing dose-dependent reduction of tracer antibody (CSL360) levels in huFcRn transgenic mice administered with varying doses of huG1FcYPY-HSA. Controls are as indicated.

FIG. 2E is a graphical representation showing dose-dependent reduction of tracer antibody (CSL360) levels in huFcRn transgenic mice administered with varying doses of huG1FcYPY-HSA (H464Q). Controls are as indicated.

FIGS. 3A and B are graphical representations showing the effect on endogenous mouse IgG levels in mice following a single administration of huG1FcYPY-HSA, huG1FcYPY-HSA (H464Q) or huG1FcYTEKF.

FIGS. 3C and D are graphical representations showing the in vivo half-life of huG1FcYPY-HSA, huG1FcYPY-HSA (H464Q) or huG1Fc YTEKF in mice.

FIG. 4A is a graphical representation showing the effect on fluorescently labeled IgG levels within bone marrow-derived macrophages in the absence or presence of FcRn antagonists huG1FcYPY-HSA, huG1FcYPY-HSA (H464Q) or huG1FcYTEKF at the concentrations and time points indicated and in the presence or absence of protease inhibitors (PI).

FIG. 4B is a graphical representation showing the effect on fluorescently labeled albumin levels within bone marrow-derived macrophages in the absence or presence of FcRn antagonists huG1FcYPY-HSA, huG1FcYPY-HSA (H464Q) or huG1FcYTEKF at the concentrations and time points indicated and in the presence or absence of protease inhibitors (PI).

FIG. 5 is a graphical representation depicting how degradation and recycling of molecules in the cell can be assessed using fluorescence.

FIG. 6 is a schematic representation depicting a putative protein complex of FcRn/β2m and huG1FcYPY-HSA (H464Q).

FIG. 7 is a schematic representation depicting the FcRn binding pocket of HSA.

FIG. 8A is a graphical representation showing the effect of FcRn antagonists on the endogenous cynomolgus monkey IgG levels up to 2016 hours (84 days) after a single intravenous administration of huG1FcYPY-HSA, huG1FcYPY-HSA (H464Q) or huG1FcYTEKF.

FIG. 8B is a graphical representation showing the effect of FcRn antagonists on the endogenous cynomolgus monkey IgG levels up to 420 hours (17.5 days) after a single intravenous administration of huG1FcYPY-HSA, huG1FcYPY-HSA (H464Q) or huG1FcYTEKF.

FIG. 8C is a graphical representation showing the in vivo PK of huG1FcYPY-HSA, huG1FcYPY-HSA (H464Q) or huG1FcYTEKF in cynomolgus monkey up to 420 hours (17.5 days) following a single intravenous administration.

FIGS. 9A-C are graphical representations of endogenous IgG and IgM levels in individual cynomolgus monkeys after a single intravenous administration of huG1FcYPY-HSA molecules.

FIGS. 9D-F are graphical representations of endogenous IgG and IgM levels in individual cynomolgus monkeys after a single intravenous administration of huG1FcYPY-HSA (H464Q) molecules.

FIGS. 9G-I are graphical representations of endogenous IgG and IgM levels in individual cynomolgus monkeys after a single intravenous administration of huG1FcYTEKF molecules.

FIG. 10A is a graphical representation showing the effect of FcRn antagonists on endogenous cynomolgus monkey IgG levels up to 2016 hours (84 days) after a single subcutaneous administration of either huG1FcYPY-HSA (H464Q) or huG1FcYTEKF.

FIG. 10B is a graphical representation showing the effect of FcRn antagonists on endogenous cynomolgus monkey IgG levels up to 420 hours (17.5 days) after a single subcutaneous administration of either huG1FcYPY-HSA (H464Q) or huG1FcYTEKF.

FIG. 10C is a graphical representation showing the in vivo half-life of huG1FcYPY-HSA (H464Q) or huG1Fc YTEKF in cynomolgus monkey up to 420 hours (17.5 days) following a single subcutaneous administration.

FIGS. 11A-C are graphical representations of endogenous IgG and IgM levels in individual cynomolgus monkeys after a single subcutaneous administration of huG1FcYPY-HSA (H464Q) molecules.

FIGS. 11D-F are graphical representations of endogenous IgG and IgM levels in individual cynomolgus monkeys after a single subcutaneous administration of huG1FcYTEKF molecules.

### KEY TO SEQUENCE LISTING

| | |
|---|---|
| SEQ ID NO: 1 | Human serum albumin (HSA) |
| SEQ ID NO: 2 | Human IgG1 Fc (huG1Fc) |
| SEQ ID NO: 3 | huG1Fc-HSA |
| SEQ ID NO: 4 | HSA-huG1Fc |
| SEQ ID NO: 5 | huG1FcYTEKF-HSA |
| SEQ ID NO: 6 | HSA-huG1FcYTEKF |
| SEQ ID NO: 7 | HSA(H464Q)-huG1FcYTEKF |
| SEQ ID NO: 8 | huG1FcYTEKF-HSA(H464Q) |
| SEQ ID NO: 9 | huG1FcYPY-HSA |
| SEQ ID NO: 10 | HSA-huG1FcYPY |
| SEQ ID NO: 11 | huG1FcYPY-HSA(H464Q) |
| SEQ ID NO: 12 | HSA(H464Q)-huG1FcYPY |
| SEQ ID NO: 13 | huG1FcYTEKF |

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

### Selected Definitions

“Albumin”, “albumin”, or “blood albumin”, is the most abundant blood protein and functions as a carrier protein for steroids, fatty acids and thyroid hormones in the blood, as well as playing a major role in stabilising extracellular fluid volume. For the purposes of nomenclature only and not limitation an exemplary sequence of a human albumin is set out in NCBI GenBank Accession ID: AEE60908 and SEQ ID NO: 1. It should be understood that reference to “albumin” or “albumin” includes preproalbumin, which comprises the N-terminal peptide, proalbumin and the secreted albumin. Positions of amino acids are referred to herein by reference to the secreted albumin protein consisting of 585 amino acids (e.g., as set out in SEQ ID NO: 1). Albumin comprises three homologous domains, wherein each domain is a product of two subdomains that possess common structural motifs. Domains I, II and III may be defined with reference to human albumin (as set forth in SEQ ID NO: 1). For example, domain I comprises amino acids 1 (+1 to 15 amino acids) to 194 (+1 to 15 amino acids) of SEQ ID NO: 1, domain II comprises amino acids 192 (+1 to 15 amino acids) to 387 (+1 to 15 amino acids) of SEQ ID NO: 1 and domain III comprises amino acid residues 381 (+1 to 15 amino acids) to 585 (+1 to 15 amino acids) of SEQ ID NO: 1. The phrase “+1 to 15 amino acids” means that the amino acid residue may deviate by 1 or 2 or 3 or 4 or 5 or 6 or 7 or 8 or 9 or 10 or 11 or 12 or 13 or 14 or 15 amino acids to the C-terminus and/or the N-terminus of the stated amino acid position. Exemplary domains I, II and III are described by Dockal et al (The Journal of Biological Chemistry, 1999, Vol. 274(41): 29303-29310) and Kjeldsen et al (Protein Expression and Purification, 1998, Vol 13:163-169).

Additional sequences of albumin from other species (e.g., primate albumin, (such as chimpanzee albumin, gorilla albumin), rodent albumin (such as hamster albumin, guinea pig albumin, mouse albumin and rat albumin), bovine albumin, equine albumin, donkey albumin, rabbit albumin, goat albumin, sheep albumin, dog albumin, chicken albumin and pig albumin) can be determined using sequences provided herein and/or in publically available databases and/or determined using standard techniques (e.g., as described in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989)).

The term “Fc” herein is used to define a C-terminal region of an immunoglobulin heavy chain that contains at least a portion of the constant region. The term includes native Fcs and Fc domain variants. In some examples, a human IgG heavy chain Fc domain region extends from Cys226, or from Pro230, to the carboxyl-terminus of the heavy chain. However, Fcs produced by host cells may undergo post-translational cleavage of one or more, particularly one or two, amino acids from the C-terminus of the heavy chain. Thus, a Fc domain may include a cleaved variant of the full-length heavy chain. This may be the case where the final two C-terminal amino acids of the heavy chain are glycine (G446) and lysine (K447, numbering according to EU index). Therefore, the C-terminal lysine (Lys447), or the C-terminal glycine (Gly446) and lysine (Lys447), of the Fc domain region may or may not be present. When specified herein, numbering of amino acid residues in the Fc domain region or constant region is according to the EU numbering system, also called the EU index, as described in Kabat et al., Sequences of Proteins of Immunological Interest, 5th Ed. Public Health Service, National Institutes of Health, Bethesda, MD, 1991.

For the purposes of nomenclature only and not limitation an exemplary sequence of a human IgG1 Fc domain is set forth in SEQ ID NO:2 or in Uniprot Accession No: P01857.

In the context of a FcRn antagonist of the present disclosure, a Fc domain or fragment thereof is capable of dimerizing with another Fc domain or fragment thereof. In one example, a FcRn antagonist of the disclosure is a homodimer.

As used herein the phrase “corresponding to” in reference to the position of an amino acid in SEQ ID NO: should be understood as reference to an amino acid residue or position within a polypeptide or protein (e.g., albumin or a Fc), and not necessarily a sequence comprising the recited SEQ ID NO. For example, reference to “a position corresponding to amino acid 522 of SEQ ID NO: 1” in an albumin sequence comprising a 10 amino acid N-terminal truncation would necessarily refer to an amino acid at position 512.

When discussing positioning of a mutation within, e.g., an albumin within a fusion protein, the numbering of the position is relative to the albumin and not relative to the entire fusion protein. Thus, if a Fc domain is fused N-terminal to the albumin, when discussing a mutation in the albumin, the first residue is the first residue of the albumin.

The term “EU numbering system of Kabat” or “EU numbering system” will be understood to mean the numbering of an immunoglobulin heavy chain is according to the EU index as taught in Kabat et al., 1991, Sequences of Proteins of Immunological Interest, 5th Ed., United States Public Health Service, National Institutes of Health, Bethesda. The EU index is based on the residue numbering of the human IgG1 EU.

As discussed herein, reference to a “fragment” of albumin should be understood as a reference to a fragment of albumin which has retained the ability to extend the half-life of a molecule to which it is conjugated and does not require that the fragment binds to FcRn. A fragment may comprise or consist of one more domains of albumin, fragments of such domains or combinations thereof.

As used herein “amino acid substitution(s)” refers to the replacement of an amino acid at a particular position in a polypeptide sequence with another amino acid.

As used herein, the term “FcRn” refers to the neonatal Fc receptor, also known as the Brambell receptor, and is a heterodimer of truncated heavy chain of the major histocompatibility complex class 1-like Fc receptor (FCGRT) and beta-2-microglobulin.

As used herein, the terms “variant” refers to a protein which has undergone substitution of one or more amino acids using well known techniques for site directed mutagenesis or any other conventional method.

As used herein, the term “binds” in reference to the interaction of a Fc domain or a fragment thereof with FcRn means that the interaction is dependent upon the presence of a particular structure on the Fc domain and the FcRn.

For the purposes of clarification and as will be apparent to the skilled artisan based on the exemplified subject matter herein, reference to “affinity” in this specification is a reference to the interaction, binding or association of an albumin or fragment thereof or Fc domain or fragment thereof with FcRn.

For the purposes of clarification and as will be apparent to the skilled artisan based on the description herein, reference to an “affinity of at least about” will be understood to mean that the affinity is equal to the recited value or higher (i.e., the value recited as the affinity is lower), i.e., an affinity of 2 nM is greater than an affinity of 3 nM. Stated another way, this term could be “an affinity of X or less”, wherein X is a value recited herein.

As used herein, the term “does not detectably bind” shall be understood to mean that an albumin variant binds to FcRn at a level less than 20%, or 10%, or 6%, or 5% above background. The background can be the level of binding signal detected in the absence of the albumin variant or FcRn comprising same and/or in the presence of a negative control protein (e.g., an isotype control antibody) and/or the level of binding detected in the presence of a negative control antigen. The level of binding is detected, for example, using ELISA in which the antigen is immobilized and contacted with an albumin variant.

As used herein, the terms “half-life”, “serum half-life” or “plasma half-life” in the context of the present disclosure refers to the period of time required for the concentration or amount of Fc domain or albumin or FcRn antagonist in the body to be reduced by 50% (i.e., one half) for example due to degradation and/or clearance or sequestration by natural mechanisms. The skilled person will recognise that the serum half-life of proteins in a subject is dependent on various physiological conditions (e.g., health status, body size/weight). For example, in a healthy human subject, the serum half-life of albumin is 19-20 days and the serum half-life of IgG1 is about 21 days. Methods for determining the serum half-life of protein are known in the art and include, for example, pharmacokinetic analysis.

The term “recombinant” shall be understood to mean the product of artificial genetic recombination. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulfide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

As used herein, the terms “treating”, “treat” or “treatment” include administering a FcRn antagonist described herein to thereby reduce or eliminate at least one symptom of a specified disease or condition or to slow progression of the disease or condition.

As used herein, the term “preventing”, “prevent” or “prevention” includes providing prophylaxis with respect to occurrence or recurrence of a bleeding disease or a symptom of a bleeding disease in an individual. An individual may be predisposed to or at risk of developing the disease or disease relapse but has not yet been diagnosed with the disease or the relapse.

An “effective amount” refers to at least an amount effective, at dosages and for periods of time necessary, to achieve the desired result. For example, the desired result may be a therapeutic or prophylactic result. An effective amount can be provided in one or more administrations. In some examples of the present disclosure, the term “effective amount” is meant an amount necessary to effect treatment of a disease or condition as hereinbefore described. In some examples of the present disclosure, the term “effective amount” is meant an amount necessary to effect a change in a factor associated with a disease or condition as hereinbefore described. The effective amount may vary according to the disease or condition to be treated or factor to be altered and also according to the weight, age, racial background, sex, health and/or physical condition and other factors relevant to the mammal being treated. Typically, the effective amount will fall within a relatively broad range (e.g., a “dosage” range) that can be determined through routine trial and experimentation by a medical practitioner. Accordingly, this term is not to be construed to limit the disclosure to a specific quantity. The effective amount can be administered in a single dose or in a dose repeated once or several times over a treatment period.

A “therapeutically effective amount” is at least the minimum concentration required to affect a measurable improvement of a particular disease or condition. A therapeutically effective amount herein may vary according to factors such as the disease state, age, sex, and weight of the patient, and the ability of the albumin conjugate to elicit a desired response in the individual. A therapeutically effective amount is also one in which any toxic or detrimental effects of the FcRn antagonist are outweighed by the therapeutically beneficial effects. In one example, a therapeutically effective amount shall be taken to mean a sufficient quantity of albumin conjugate to reduce or inhibit one or more symptoms of a bleeding disorder or a complication thereof.

As used herein, the term “prophylactically effective amount” shall be taken to mean a sufficient quantity of the albumin conjugate to prevent or inhibit or delay the onset of one or more detectable symptoms of a condition.

As used herein, the term “subject” shall be taken to mean any animal including humans, for example a mammal. Exemplary subjects include but are not limited to humans and non-human primates. For example, the subject is a human.

### Fc Domain and Fragments Thereof

As described herein, a FcRn antagonist of the disclosure comprises a Fc domain or FcRn binding fragment thereof.

In one example, the Fc domain is from IgG.

For example, the Fc domain is from human IgG.

For example, the Fc domain is from IgG1.

For example, the Fc domain is from human IgG1.

For example, the Fc domain is from IgG4.

For example, the Fc domain is from human IgG4.

For example, the Fc domain is not from IgG4.

For example, the Fc domain is not from human IgG4.

In one example, the FcRn binding fragment of the Fc domain refers to the part of an immunoglobulin heavy chain, e.g., IgG1 heavy chain, that extends approximately from EU position 243 to EU position 261 and approximately from EU position 275 to EU position 293 and approximately from EU position 302 to EU position 319 and approximately from EU position 336 to EU position 348 and approximately from EU position 367 to EU position 393 and EU position 408 and approximately from EU position 424 to EU position 440.

In certain examples, the Fc domain or fragment are not full-length immunoglobulins. For example, the Fc domain or fragment or FcRn antagonist do not comprise a variable domain. In some examples, the Fc domain or fragment or FcRn antagonist do not comprise a variable domain or a C_{H}1 domain. However, in certain examples, the FcRn antagonist may comprise a Fc domain or fragment thereof linked to one or more additional binding domains or moieties, including variable domains.

In one example, the Fc domain is a Fc domain variant, or FcRn-binding fragment thereof, e.g., that binds specifically to FcRn with increased affinity compared to a Fc domain from wild-type human IgG1. In one example, the variant Fc domain or fragment thereof has reduced pH dependence for binding to FcRn relative to a native Fc domain region. An exemplary Fc domain or fragments thereof inhibits or reduces the binding of immunoglobulins and/or other Fc-containing proteins (e.g. immunoadhesins and antibody-drug conjugates) to FcRn in vivo, which results in an increased rate of degradation of the immunoglobulin or Fc-containing proteins and, concomitantly, a reduced serum level of these immunoglobulins or Fc-containing proteins.

Any Fc domain region can be modified to produce a Fc domain variant of the disclosure. As discussed herein, generally a Fc domain is from a human immunoglobulin. However, the Fc domain may be derived from an immunoglobulin of any other mammalian species, including for example, a Camelid species, a rodent (e.g. a mouse, rat, rabbit, guinea pig) or non-human primate (e.g. chimpanzee, macaque) species. Moreover, the Fc domain may be derived from any immunoglobulin class, including IgM, IgG, IgD, IgA and IgE, and any immunoglobulin isotype, including IgG1, IgG2, IgG3 and IgG4. In certain examples, the Fc domain is an IgG Fc domain (e.g., a human IgG region). In certain examples, the Fc domain is an IgG1 Fc domain (e.g., a human IgG1). In certain examples, the Fc domain is a chimeric Fc domain comprising portions of several different Fc domain. Suitable examples of a chimeric Fc domain is set forth in US20110243966. It will be appreciated that the scope of the present disclosure encompasses alleles, variants and mutations of Fc domains.

A Fc domain can be further truncated or include a deletion to produce a minimal FcRn-binding fragment thereof. The ability of a Fc domain fragment to bind to FcRn can be determined using any art recognized binding assay e.g., ELISA.

To enhance the manufacturability of the FcRn antagonists disclosed herein, it is desirable that the constituent Fc domain does not comprise any non-disulphide bonded cysteine residues. Accordingly, in certain examples the Fc domains do not comprise a free cysteine residue.

In one example, the Fc domain variant comprises an amino acid modification, e.g., substitution that enhances binding of the Fc domain to FcRn. In one example, the amino acid modification enhances binding of the Fc domain to FcRn at neutral pH and/or acidic pH. In one example, the amino acid modification enhances binding of the Fc domain to FcRn at neutral pH and acidic pH.

In some examples, the Fc domain variant comprises a substitution at one or more positions selected from the group consisting of 234, 235, 236, 239, 240, 241, 243, 244, 245, 247, 252, 254, 256, 262, 263, 264, 265, 266, 267, 269, 296, 297, 298, 299, 313, 325, 326, 327, 328, 329, 330, 332, 333, and 334 according to the EU numbering system. Some exemplary substitutions are described in U.S. Pat. Nos. 5,624,821; 6,277,375; 6,737,056; WO01/58957; WO02/06919; WO2004/016750; WO2004/029207; WO 2004/035752; WO2005/040217 and WO2015/100299.

In some examples, the Fc domain variant comprises at least one substitution selected from the group consisting of 234D, 234E, 234N, 234Q, 234T, 234H, 234Y, 2341, 234V, 234F, 235A, 235D, 235R, 235W, 235P, 235S, 235N, 235Q, 235T, 235H, 235Y, 2351, 235V, 235F, 236E, 239D, 239E, 239N, 239Q, 239F, 239T, 239H, 239Y, 2401, 240A, 240T, 240M, 241W, 241L, 241Y, 241E, 241R. 243W, 243L 243Y, 243R, 243Q, 244H, 245A, 247V, 247G, 252Y, 254T, 256E, 2621, 262A, 262T, 262E, 2631, 263A, 263T, 263M, 264L, 2641, 264W, 264T, 264R, 264F, 264M, 264Y, 264E, 265G, 265N, 265Q, 265Y, 265F, 265V, 2651, 265L, 265H, 265T, 2661, 266A, 266T, 266M, 267Q, 267L, 269G, 269H, 269Y, 269F, 269R, 296E, 296Q, 296D, 296N, 296S, 296T, 296L, 2961, 296H, 296G, 296W, 297S, 297D, 297E, 298H, 2981, 298T, 298F, 2991, 299L, 299A, 299S, 299V, 299H, 299F, 299E, 313F, 325Q, 325L, 3251, 325D, 325E, 325A, 325T, 325V, 325H, 327G, 327W, 327N, 327L, 328S, 328M, 328D, 328E, 328N, 328Q, 328F, 3281, 328V, 328T, 328H, 328A, 329F, 329H, 329Q, 330K, 330G, 330T, 330C, 330L, 330Y, 330V, 3301, 330F, 330R, 330H, 332D, 332S, 332W, 332F, 332E, 332N, 332Q, 332T, 332H, 332Y, 332A. 334N, 352S, 378V, 397M and 434Y according to the EU numbering system.

Other known Fc domain variants that may be used in the FcRn antagonists disclosed herein include without limitation those disclosed in Ghetie et al, 1997, Nat. Biotech. 15:637-40; Duncan et al, 1988, Nature 332:563-564; Lund et al, 1991, J. Immunol, 147:2657-2662; Lund et al, 1992, Mol. Immunol, 29:53-59; Alegre et al, 1994, Transplantation 57:1537-1543; Hutchins et al, 1995, Proc Natl. Acad Sci USA, 92:11980-11984; Jefferis et al, 1995, Immunol Lett., 44:111-117; Lund et al, 1995, Faseb J., 9:115-119; Jefferis et al, 1996, Immunol Lett., 54:101-104; Lund et al, 1996, J. Immunol, 157:4963-4969; Armour et al, 1999, Eur J Immunol 29:2613-2624; Idusogie et al, 2000, J. Immunol, 164:4178-4184; Reddy et al, 2000, J. Immunol, 164:1925-1933; Xu et al, 2000, Cell Immunol, 200:16-26; Idusogie et al, 2001, J. Immunol, 166:2571-2575; Shields et al, 2001, J Biol. Chem., 276:6591-6604; Jefferis et al, 2002, Immunol Lett., 82:57-65; Presta et al, 2002, Biochem Soc Trans., 30:487-490.

Additional exemplary substitutions will be apparent to the skilled person. For example, substitutions at residues 250 and 428 (according to the EU numbering system), e.g., T250Q and M428L have been shown to increase binding of a Fc domain to FcRn (Hinton, et al., J. Biol. Chem. 279, 6213-6216, 2004). Similarly, substitutions at residues 252, 254 and 256 (according to the EU numbering system), e.g., M252Y, S254T and T256E have been shown to increase binding of a Fc domain to FcRn (Dall'Acqua, et al., J Immunol 169, 5171-5180, 2002 and Dall'Acqua, et al., The Journal of Biological Chemistry 281, 23514-23524, 2006). Dall'Acqua et al., 2002 additionally teaches the following combinations of residues are useful for enhancing binding of a Fc domain to FcRn M252W or M252Y or M252Y/T256Q or M252F/T256D or V203T/L309P/Q311S or H433K/N434F/Y436H or H433R/N434Y/Y436H or M252Y/S254T/T256E/H433K/N434F/Y436H or M252Y/S254T/T256E/G385R/Q386T/P387R/N389P (according to the EU numbering system). Additional substitutions that enhance binding of Fc domain to FcRn include T307A/E380A/N434A or M428L/N434S.

Mackness et al., (MABS 11:1276-1288, 2019) showed the following substitutions bound to FcRn and had substantially reduced off-rates compared to wild-type Fc: M252Y, T256D/E, K288D/N, T307A/E/F/M/Q/W, E380C, N434F/P/Y and Y436H/N/W. The authors showed that the M252Y/T256D, T256D/T307Q and T256D/T307W combinations of substitutions showed enhanced binding to FcRn while maintaining other activities, e.g., thermal stability and the FcγRIIIa and rheumatoid factor binding.

Monnet et al., (Front. Immunol, 12:728322, 2021) showed the following substitutions showed improved binding to FcRn: Y296W/K334N/P352S/A378V/V397M/N434Y.

Additional substitutions to Fc domains are described in WO2010/106180; WO2016177984; WO2018007453; WO2018078138; WO2017006052; and WO2019115773.

WO2015/100299 describes substitutions at residues 252, 254, 256, 433, 434, and 436 (according to the EU numbering system) that increase the binding of a Fc domain to FcRn. For example, the Fc domain is the same as efgartigimod.

Additional exemplary substitutions are described, for example, in WO2002/060919, which describes substitutions at residues 251, 253, 255, 285-290, 308-314, 385-389, and 428-435 (according to the EU numbering system), that increase binding of a Fc domain to FcRn.

In one example, the Fc domain variant or fragment thereof comprises one or more amino acid substitutions selected from the group consisting of:
(i) an amino acid substituted for methionine at a position corresponding to amino acid 252 according to the EU numbering system;
(ii) an amino acid substituted for serine at a position corresponding to amino acid 254 according to the EU numbering system;
(iii) an amino acid substituted for threonine at a position corresponding to amino acid 256 according to the EU numbering system;
(iv) an amino acid substituted for asparagine at a position corresponding to amino acid 286 according to the EU numbering system;
(v) an amino acid substituted for tyrosine at a position corresponding to amino acid 296 according to the EU numbering system;
(vi) an amino acid substituted for valine at a position corresponding to amino acid 308 according to the EU numbering system;
(vii) an amino acid substituted for lysine at a position corresponding to amino acid 334 according to the EU numbering system;
(viii) an amino acid substituted for proline at a position corresponding to amino acid 352 according to the EU numbering system;
(ix) an amino acid substituted for alanine at a position corresponding to amino acid 378 according to the EU numbering system;
(x) an amino acid substituted for valine at a position corresponding to amino acid 397 according to the EU numbering system;
(xi) an amino acid substituted for histidine at a position corresponding to amino acid 433 according to the EU numbering system;
(xii) an amino acid substituted for asparagine at a position corresponding to amino acid 434 according to the EU numbering system; and
(xiii) combinations thereof.

In one example, the Fc domain variant or fragment thereof comprises one or more amino acid substitutions selected from the group consisting of:
(i) tyrosine substituted for methionine at a position corresponding to amino acid 252 according to the EU numbering system;
(ii) threonine substituted for serine at a position corresponding to amino acid 254 according to the EU numbering system;
(iii) glutamic acid substituted for threonine at a position corresponding to amino acid 256 according to the EU numbering system;
(iv) glutamic acid substituted for asparagine at a position corresponding to amino acid 286 according to the EU numbering system;
(v) tryptophan substituted for tyrosine at a position corresponding to amino acid 296 according to the EU numbering system;
(vi) proline substituted for valine at a position corresponding to amino acid 308 according to the EU numbering system;
(vii) asparagine substituted for lysine at a position corresponding to amino acid 334 according to the EU numbering system;
(viii) serine substituted for proline at a position corresponding to amino acid 352 according to the EU numbering system;
(ix) valine d substituted for alanine at a position corresponding to amino acid 378 according to the EU numbering system;
(x) methionine substituted for valine at a position corresponding to amino acid 397 according to the EU numbering system;
(xi) lysine substituted for histidine at a position corresponding to amino acid 433 according to the EU numbering system;
(xii) tyrosine substituted for asparagine at a position corresponding to amino acid 434 according to the EU numbering system;
(xiii) phenylalanine substituted for asparagine at a position corresponding to amino acid 434 according to the EU numbering system; and
(xiv) combinations thereof.

In one example, the Fc domain variant or fragment thereof comprises a substitution at each of the following positions 252, 308 and 434.

For example, the Fc domain variant or fragment thereof comprises:
(i) tyrosine substituted for methionine at a position corresponding to amino acid 252 according to the EU numbering system;
(ii) proline substituted for valine at a position corresponding to amino acid 308 according to the EU numbering system; and
(iii) tyrosine or phenylalanine substituted for asparagine at a position corresponding to amino acid 434 according to the EU numbering system;
In one example, the Fc domain variant or fragment thereof comprises a substitution at each of the following positions 252, 254 and 256.

For example, the Fc domain variant or fragment thereof comprises:
(i) tyrosine substituted for methionine at a position corresponding to amino acid 252 according to the EU numbering system;
(ii) threonine substituted for serine at a position corresponding to amino acid 254 according to the EU numbering system; and
(iii) glutamic acid substituted for threonine at a position corresponding to amino acid 256 according to the EU numbering system.

In one example, the Fc domain variant or fragment thereof comprises a substitution at each of the following positions 252, 286 and 434.

For example, the Fc domain variant or fragment thereof comprises:
(i) tyrosine substituted for methionine at a position corresponding to amino acid 252 according to the EU numbering system;
(ii) glutamic acid substituted for asparagine at a position corresponding to amino acid 286 according to the EU numbering system; and
(iii) tyrosine or phenylalanine substituted for asparagine at a position corresponding to amino acid 434 according to the EU numbering system.

In one example, the Fc domain variant or fragment thereof comprises a substitution at each of the following positions 252, 254, 256, 433 and 434. For example, the Fc domain variant or fragment thereof comprises:
(i) tyrosine substituted for methionine at a position corresponding to amino acid 252 according to the EU numbering system;
(ii) threonine substituted for serine at a position corresponding to amino acid 254 according to the EU numbering system;
(iii) glutamic acid substituted for threonine at a position corresponding to amino acid 256 according to the EU numbering system;
(iv) lysine substituted for histidine at a position corresponding to amino acid 433 according to the EU numbering system; and
(v) tyrosine or phenylalanine substituted for asparagine at a position corresponding to amino acid 434 according to the EU numbering system.

In one example, the Fc domain variant or fragment thereof comprises a substitution at each of the following positions 296, 334, 352, 378, 397 and 434.

For example, the Fc domain variant or fragment thereof comprises:
(i) tryptophan substituted for tyrosine at a position corresponding to amino acid 296 according to the EU numbering system;
(ii) asparagine substituted for lysine at a position corresponding to amino acid 334 according to the EU numbering system;
(iii) serine substituted for proline at a position corresponding to amino acid 352 according to the EU numbering system;
(iv) valine substituted for alanine at a position corresponding to amino acid 378 according to the EU numbering system;
(v) methionine substituted for valine at a position corresponding to amino acid 397 according to the EU numbering system; and
(vi) tyrosine substituted for asparagine at a position corresponding to amino acid 434 according to the EU numbering system.

In one example, the Fc domain variant or fragment thereof comprises a substitution at each of the following positions 252, 254, 256, 433 and 434.

For example, the Fc domain variant or fragment thereof comprises:
(i) tyrosine substituted for methionine at a position corresponding to amino acid 252 according to the EU numbering system;
(ii) threonine substituted for serine at a position corresponding to amino acid 254 according to the EU numbering system;
(iii) glutamic acid substituted for threonine at a position corresponding to amino acid 256 according to the EU numbering system;
(iv) lysine substituted for histidine at a position corresponding to amino acid 433 according to the EU numbering system; and
(v) phenylalanine substituted for asparagine at a position corresponding to amino acid 434 according to the EU numbering system.

In one exemplary form of the disclosure, the Fc domain variant or fragment thereof comprises a substitution at each of the following positions 252, 254, 256, 433, 434, and 436.

For example, the Fc domain variant or fragment thereof comprises:
(i) tyrosine substituted for methionine at a position corresponding to amino acid 252 according to the EU numbering system;
(ii) threonine substituted for serine at a position corresponding to amino acid 254 according to the EU numbering system;
(iii) glutamic acid substituted for threonine at a position corresponding to amino acid 256 according to the EU numbering system;
(iv) lysine substituted for histidine at a position corresponding to amino acid 433 according to the EU numbering system;
(v) tyrosine or phenylalanine substituted for asparagine at a position corresponding to amino acid 434 according to the EU numbering system;
(vi) histidine substituted for tyrosine at a position corresponding to amino acid 436 according to the EU numbering system; and
(vii) combinations thereof.

In one example, the Fc domain variant or fragment thereof comprises:
(i) tyrosine substituted for methionine at a position corresponding to amino acid 252 according to the EU numbering system, proline substituted for valine at a position corresponding to amino acid 308 according to the EU numbering system and tyrosine substituted for asparagine at a position corresponding to amino acid 434 according to the EU numbering system; or
(ii) tyrosine substituted for methionine at a position corresponding to amino acid 252 according to the EU numbering system, threonine substituted for serine at a position corresponding to amino acid 254 according to the EU numbering system and glutamic acid substituted for threonine at a position corresponding to amino acid 256 according to the EU numbering system; or
(iii) tyrosine substituted for methionine at a position corresponding to amino acid 252 according to the EU numbering system, glutamic acid substituted for asparagine at a position corresponding to amino acid 286 according to the EU numbering system and tyrosine substituted for asparagine at a position corresponding to amino acid 434 according to the EU numbering system; or
(iv) tyrosine substituted for methionine at a position corresponding to amino acid 252 according to the EU numbering system, threonine substituted for serine at a position corresponding to amino acid 254 according to the EU numbering system, glutamic acid substituted for threonine at a position corresponding to amino acid 256 according to the EU numbering system, lysine substituted for histidine at a position corresponding to amino acid 433 according to the EU numbering system and phenylalanine substituted for asparagine at a position corresponding to amino acid 434 according to the EU numbering system.

In one example, the Fc domain variant or fragment thereof comprises tyrosine substituted for methionine at a position corresponding to amino acid 252 according to the EU numbering system, threonine substituted for serine at a position corresponding to amino acid 254 according to the EU numbering system, glutamic acid substituted for threonine at a position corresponding to amino acid 256 according to the EU numbering system, lysine substituted for histidine at a position corresponding to amino acid 433 according to the EU numbering system and phenylalanine substituted for asparagine at a position corresponding to amino acid 434 according to the EU numbering system.

In some examples, the Fc domain variant has altered (e.g., increased or decreased) binding affinity for an additional Fc receptor. The Fc domain variant can have altered (e.g., increased or decreased) binding affinity for one or more of Fcy receptors e.g., FcγRI(CD64), FcγRIIA(CD32), FcγRIIB(CD32), FcγRIIIA(CD 16a), and FcγRIIIB (CD 16b). Any art recognized means of altering the affinity for an additional Fc receptor can be employed. In certain examples, the amino acid sequence of the variant Fc domain is altered.

In some examples, the Fc domain or fragment thereof forms a homodimer.

In other examples, the Fc domain or fragment thereof forms a heterodimer. Methods of producing Fc domain heterodimers are known in the art (see e.g., U.S. Pat. No. 8,216,805). In one example, the Fc domain heterodimer is produced using “knob and hole” or “key and hole” technology. For example, one Fc domain in the heterodimer comprises at least one amino acid substitution that results in at least one side chain to be exposed and form the “knob” and the other Fc domain in the heterodimer comprises at least one amino acid substitution to form a cavity that can accept the side-chain, i.e., hole. For example, a Fc domain comprises a T366W mutation (or knob) and another Fc domain comprises a T366S, L368A and Y407V mutation (or hole). In another example, a first Fc domain comprises T350V, T366L, K392L and T394W mutations (knob) and a second Fc domain comprises T350V, L351Y, F405A and Y407V mutations (hole). Using such technology, a FcRn antagonist can be formed with a single albumin or with one albumin and another protein. For example, one Fc domain in the heterodimer is linked to the albumin and the other Fc domain is either not linked to any additional protein or is linked to a protein other than albumin.

In some examples, the Fc domain variant is a single chain Fc, where the constituent Fc domain regions are linked together by a linker. Methods of producing single chain Fc domain regions are known in the art (see e.g., US20090252729 and US20110081345).

As discussed herein, pathogenic IgG observed in autoimmune diseases are either the pathogenic triggers for these diseases or contribute to disease progression and mediate disease through the inappropriate activation of cellular Fc receptors. Aggregated autoantibodies and/or autoantibodies complexed with self-antigens (immune complexes) bind to activating Fc receptors, causing numerous autoimmune diseases (which occur in part because of immunologically mediated inflammation against self-tissues). Accordingly, to treat antibody-mediated disorders (e.g. autoimmune diseases), it would be advantageous to both remove the deleterious autoantibodies and to block the interaction of the immune complexes of these antibodies with activating Fc receptors (e.g., Fcγ receptors, such as CD16a).

In some examples, the Fc domain variants of the FcRn antagonist exhibit increased binding to CD16a (e.g., human CD16a) compared to the corresponding wildtype Fc. In certain examples, the FcRn antagonist comprises a variant Fc-region comprising an N-linked glycan (e.g., at EU position 297). In this case, it is possible to increase the binding affinity of the FcRn antagonist for CD16a by altering the glycan structure. Alterations of the N-linked glycan of Fc domains are known in the art. For example, afucosylated N-linked glycans or N-glycans having a bisecting GlcNac structure have been shown to exhibit increased affinity for CD16a. Accordingly, in certain examples, the N-linked glycan is afucosylated. Afucosylation can be achieved using any art recognized means. For example, an FcRn antagonist can be expressed in cells lacking fucosyl transferase, such that fucose is not added to the N-linked glycan at EU position 297 of the variant Fc domain (see e.g., U.S. Pat. No. 8,067,232, the contents of which is incorporated by reference herein in its entirety). In certain examples, the N-linked glycan has a bisecting GlcNac structure. The bisecting GlcNac structure can be achieved using any art recognized means.

In other examples, the Fc domain variants of the FcRn antagonist exhibit decreased binding to CD16a (e.g., human CD16a).

In other examples, the Fc domain variants of the FcRn antagonist exhibit similar binding to CD16a (e.g., human CD16a) compared to the corresponding wildtype Fc.

In some examples, a FcRn antagonist described herein is monomeric. For example, the Fc domain comprises one or more mutations reducing or preventing dimerization. Exemplary mutations are described in WO2011/063348; WO2018/144784; WO2013/138643; WO2014/087299; and WO2013/166604.

### Albumin and Fragments Thereof

The FcRn antagonists of the present disclosure include albumin or fragments thereof capable of extending the half-life of the antagonist. In one example, the albumin comprises a sequence set forth in SEQ ID NO: 1. In one example, an albumin of the present disclosure comprises a sequence at least about 85% or 90% or 95% or 97% or 98% or 99% identical to a sequence disclosed herein.

In one example, the albumin is human serum albumin (HSA).

In one example, the human albumin variant or fragment thereof is a naturally-occurring albumin variant. Exemplary substitutions to a naturally-occurring albumin variant are described, for example, in Peach & Brenan (BBA 1097:49-54, 1991), Iwao et al. (BBA Proteins & Proteomics 1774:1582-90, 2007) and Galliano et al. (BBA 1225:27-32, 1993).

Peach & Brenan shows various substitutions to a naturally-occurring albumin variant. Exemplary substitutions include at position 494 (e.g., D494N).

Iwao et al. shows various substitutions to a naturally-occurring albumin variant. Exemplary substitutions include at position 541 (e.g., K541E) or at position 560 (e.g., K560E) or at position 501 (e.g., E501K) or at position 570 (e.g., E570K) or at position 573 (e.g., K573E).

Galliano et al. shows various substitutions to a naturally-occurring albumin variant. Exemplary substitutions include at position 505 (e.g., E505K).

In one example, the fragment of albumin is capable of extending the half-life of the FcRn antagonist without necessarily binding to FcRn. In one example, the fragment of albumin binds to FcRn with a reduced affinity compared to wild-type albumin. In another example, the fragment of albumin does not detectably bind to FcRn.

In some examples, the albumin is an albumin variant. In some examples, the albumin variant binds to FcRn with a reduced affinity compared to wild-type albumin. In some examples, the albumin variant does not detectably bind to FcRn. In some examples, the binding of albumin to FcRn is determined by immobilizing soluble FcRn on a soluble surface and detecting binding of albumin using SPR. In some examples, the albumin is immobilized and the binding of the soluble FcRn is determined using SPR.

In one example, the albumin variant comprises an amino acid modification, e.g., substitution that reduces binding of the albumin to FcRn. Substitutions that reduce binding of albumin to FcRn are known in the art and described, for example, in WO2012/150319; WO2011/051489; U.S. Pat. No. 10,696,732; or U.S. Pat. No. 8,822,417.

Exemplary substitutions to albumin are described herein and include a substitution at position 464 (e.g., H464Q) or position 510 (e.g., H510Q or H510R) or position 535 (e.g., H535Q or H535F) relative to SEQ ID NO: 1.

WO2011/051489 shows that various substitutions to albumin reduce affinity for FcRn. Exemplary substitutions include at position 494 (e.g., D494Q, D494N or D494A) or position 495 (e.g., E495Q or E495A) or position 496 (e.g., T496A) or position 417 (e.g., Q417A) or position 499 (e.g., P499A) or position 536 (e.g., K536A) or position 538 (e.g., K538A) or position 550 (e.g., D550N). Additionally, combinations of substitutions reduce affinity for FcRn, e.g., at position 494 and 496 (e.g., D494N and T496A) or position 494 and 417 (e.g., D494E and Q417H).

WO2012/150319 also shows various substitutions to albumin reduce affinity for FcRn. Exemplary substitutions include at position 500 (K500A) or position 417 (e.g., Q417A) or position 536 (e.g., K536A) or position 537 (e.g., P537A) or position 538 (e.g., K538A) or position 573 (e.g., K573P) or position 580 (Q580A) or position 111 (e.g., N111H; N111K; N111E) or position 512 (e.g., D512E) or position 527 (e.g., T527A) or position 569 (e.g., A569S) or position 108 (e.g., D108A).

U.S. Pat. No. 10,696,732 also shows various substitutions to albumin reduce affinity for FcRn. Exemplary substitutions include at position 494 (e.g., D494N; D494A; D494Q) or position 496 (e.g., T496A) or position 417 (e.g., Q417A), or position 499 (e.g., P499A) or position 500 (e.g., K500A) or position 536 (e.g., K536A) or position 537 (e.g., K537A) or position 538 (e.g., K538A) or position 501 (e.g., E501A or 501Q).

Schmidt et al. (Structure 21, 1966-1978, 2013) also shows various substitutions to albumin reduce affinity for FcRn.

In one example, the albumin variant or fragment thereof comprises one or more amino acid substitutions selected from the group consisting of:
(i) an amino acid substituted for histidine at a position corresponding to amino acid 464 of SEQ ID NO: 1;
(ii) an amino acid substituted for threonine at a position corresponding to amino acid 422 of SEQ ID NO: 1;
(iii) an amino acid substituted for histidine at a position corresponding to amino acid 510 of SEQ ID NO: 1;
(iv) an amino acid substituted for histidine at a position corresponding to amino acid 535 of SEQ ID NO: 1; and
(v) combinations thereof.

In one example, the albumin variant or fragment thereof comprises glutamine substituted for histidine at a position corresponding to amino acid 464 of SEQ ID NO: 1.

In one example, the albumin variant or fragment thereof comprises tryptophan substituted for threonine at a position corresponding to amino acid 422 of SEQ ID NO: 1.

In one example, the albumin variant or fragment thereof comprises glutamine substituted for histidine at a position corresponding to amino acid 510 of SEQ ID NO: 1.

In one example, the albumin variant or fragment thereof comprises arginine substituted for histidine at a position corresponding to amino acid 510 of SEQ ID NO: 1.

In one example, the albumin variant or fragment thereof comprises phenylalanine substituted for histidine at a position corresponding to amino acid 535 of SEQ ID NO: 1.

In one example, the albumin variant or fragment thereof comprises glutamine substituted for histidine at a position corresponding to amino acid 535 of SEQ ID NO: 1.

In one example, the albumin variant or fragment comprises an amino acid substitution at a position corresponding to amino acid 463 of SEQ ID NO: 1.

In one example, the albumin variant or fragment comprises an amino acid substitution at a position corresponding to amino acid 467 of SEQ ID NO: 1.

In one example, the albumin variant or fragment comprises an amino acid substitution at a position corresponding to amino acid 509 of SEQ ID NO: 1.

In one example, the albumin variant or fragment comprises an amino acid substitution at a position corresponding to amino acid 519 of SEQ ID NO: 1.

In one example, the albumin variant or fragment comprises glutamine substituted for histidine at a position corresponding to amino acid 464 of SEQ ID NO: 1.

In one example, the albumin variant or fragment comprises tryptophan substituted for threonine at a position corresponding to amino acid 422 of SEQ ID NO: 1, and glutamine substituted for histidine at a position corresponding to amino acid 464 of SEQ ID NO: 1.

In one example, the albumin variant or fragment comprises glutamine substituted for histidine at a position corresponding to amino acid 464 of SEQ ID NO: 1 and phenylalanine substituted for histidine at a position corresponding to amino acid 535 of SEQ ID NO: 1.

In one example, the albumin variant or fragment thereof comprises arginine substituted for histidine at a position corresponding to amino acid 510 of SEQ ID NO: 1 and phenylalanine substituted for histidine at a position corresponding to amino acid 535 of SEQ ID NO: 1.

Exemplary methods for producing variant forms of albumin are described herein or known in the art and include:
mutagenesis of DNA (Thie et al., Methods Mol. Biol. 525:309-322, 2009) or RNA (Kopsidas et al., Immunol. Lett. 107:163-168, 2006; Kopsidas et al. BMC Biotechnology, 7:18, 2007; and WO1999/058661);
introducing a nucleic acid encoding the polypeptide into a mutator cell, e.g., XL-1Red, XL-mutS and XL-mutS-Kanr bacterial cells (Stratagene);
DNA shuffling, e.g., as disclosed in Stemmer, Nature 370:389-91, 1994; and
site directed mutagenesis, e.g., as described in Dieffenbach (ed) and Dveksler (ed) (In: PCR Primer: A Laboratory Manual, Cold Spring Harbor Laboratories, NY, 1995).

Exemplary methods for determining biological activity of the albumin variant, or fragment thereof, of the disclosure will be apparent to the skilled person and/or described herein, e.g., FcRn affinity. For example, methods for determining affinity of the albumin variant, or fragment thereof, include affinity, association, dissociation and therapeutic efficacy are described herein.

In some examples, a FcRn antagonist comprises more than one albumin. In some examples, more than one albumin is linked to a Fc domain. For example, within a FcRn antagonist, a single Fc domain is linked to more than one albumin or fragment thereof. In some examples, the albumins are linked in series, e.g., to the C-terminus of the Fc domain. In other examples, one or more albumins or fragments thereof are linked to the N-terminus of the Fc domain and one or more albumins or fragments thereof are linked to the C-terminus of the Fc domain. In some examples, the Fc domain can dimerize, thus further increasing the number of albumins in the FcRn antagonist.

In one example, each Fc domain in a FcRn antagonist is linked to a single albumin. As exemplified herein linking an albumin or fragment thereof to the C-terminus of the Fc domain provides for extended half-life and increased FcRn antagonism.

### Linkers

In some examples, components of the FcRn antagonist of the disclosure are indirectly linked, e.g., via a linker. In some examples, the linker is a polypeptide linker.

In some examples, a polypeptide linker comprises or consists of a gly-ser linker. As used herein, the term “gly-ser linker” refers to a peptide that consists of glycine and serine residues. An exemplary gly/ser linker comprises an amino acid sequence of the formula (Gly₄Ser)ₙ, wherein n is a positive integer (e.g., 1, 2, 3, 4, or 5). In certain examples the gly/ser linker is (Gly₄Ser)₁. In some examples, the gly/ser linker is (Gly₄Ser)₂. In some examples the gly/ser linker is (Gly₄Ser)₃ or (Gly₄Ser)₄.

Other linkers that are suitable for use in the FcRn antagonists of the disclosure are known in the art, for example, the serine-rich linkers disclosed in U.S. Pat. No. 5,525,491, the helix forming peptide linkers (e.g., A(EAAAK)nA (n=2-5)) disclosed in Arai et al, Protein Eng 2001; 14:529-32, or the stable linkers disclosed in Chen et al, Mol Pharm 2011; 8:457-65.

Other exemplary linkers include GS linkers (i.e., (GS)n), GGSG linkers (i.e., (GGSG)n), GSAT linkers, SEG linkers, and GGS linkers (i.e., (GGSGGS)n), wherein n is a positive integer (e.g., 1, 2, 3, 4, or 5).

Polypeptide linkers of the disclosure are at least one amino acid in length and can be of varying lengths. In some examples, a polypeptide linker of the disclosure is from about 1 to about 50 amino acids in length. In another example, a polypeptide linker of the disclosure is from about 5-10 amino acids in length. In another example, a polypeptide linker of the disclosure is from about 10-20 amino acids in length. In another example, a polypeptide linker of the disclosure is from about 15 to about 50 amino acids in length.

In some example, the linker comprises or is a chemical linker. In some examples, the linker is one or more ethylene glycol (EG) units, e.g., 2 or more EG units (i.e., polyethylene glycol (PEG)). In some examples, a linker comprises or consists of a polyethylene glycol (PEG) linker. Polyethylene glycol or PEG refers to a chemical compound composed of repeating ethylene glycol units. An exemplary “PEG linker” comprises a compound of the formula: H-(0-CH2-CH2)n-OH, wherein n is a positive integer (e.g., 1, 10, 20, 50, 100, 200, 300, 400, 500, 600). In some examples, the PEG linker is PEG1000. In some examples, the PEG linker is PEG2000. In some examples, the PEG linker is PEG3000.

In some examples, a FcRn antagonist comprises a Fc domain or fragment thereof linked to a PEG which is in turn linked to an albumin or fragment thereof.

As discussed herein, the N-terminus of the Fc domain can be linked to the N-terminus of the albumin or the C-terminus of the Fc domain can be linked to the C-terminus of the albumin. Chemical linkers are suited to such linkages.

### Production of FcRn Antagonists

The disclosure provides polynucleotides, vectors and host cells encoding the FcRn antagonists disclosed herein. Methods of making an FcRn antagonists comprising expressing these polynucleotides are also provided.

Polynucleotides encoding the FcRn antagonists disclosed herein are typically inserted in an expression vector for introduction into host cells that may be used to produce the desired quantity of the claimed FcRn antagonists. Accordingly, in some examples, the disclosure provides expression vectors comprising polynucleotides disclosed herein and host cells comprising these vectors and polynucleotides.

The term “vector” or “expression vector” is used herein for the purposes of the specification and claims, to mean vectors used in accordance with the present disclosure as a vehicle for introducing into and expressing a desired gene in a cell. As known to those skilled in the art, such vectors may easily be selected from the group consisting of plasmids, phages, viruses and retroviruses.

Numerous expression vector systems may be employed for the purposes of this disclosure. For example, one class of vector utilizes DNA elements, which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells that have integrated the DNA into their chromosomes may be selected by introducing one or more markers that allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements may also be needed for optimal synthesis of mRNA. These elements may include signal sequences, splice signals, as well as transcriptional promoters, enhancers, and termination signals.

More generally, once a vector or DNA sequence encoding an FcRn antagonist has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Most preferably, plasmid introduction into the host is via electroporation. The transformed cells are grown under conditions appropriate to the production of the FcRn antagonist, and assayed for FcRn antagonist expression. Exemplary assay techniques include enzyme-linked immunosorbent assay (ELISA), radioimmunoassay (RIA), or flourescence-activated cell sorter analysis (FACS), immunohistochemistry and the like.

In vitro production allows scale-up to give large amounts of the desired FcRn antagonist. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

Genes encoding the FcRn antagonists of the disclosure can also be expressed in non-mammalian cells such as bacteria or yeast or plant cells. In this regard it will be appreciated that various unicellular non-mammalian microorganisms such as bacteria can also be transformed; i.e. those capable of being grown in cultures or fermentation. Bacteria, which are susceptible to transformation, include members of the enterobacteriaceae, such as strains of Escherichia coli or Salmonella. It will further be appreciated that, when expressed in bacteria, the FcRn antagonists can become part of inclusion bodies. The FcRn antagonists must be isolated, purified and then assembled into functional molecules. In addition to prokaryotes, eukaryotic microbes may also be used. Saccharomyces cerevisiae, or common baker's yeast, is the most commonly used among eukaryotic microorganisms although a number of other strains are commonly available.

### Assaying Activity of a FcRn Antagonist

FcRn antagonists of the present disclosure are readily screened for biological activity, e.g., as described below.

### Determining Affinity

Optionally, the dissociation constant (Kd) or association constant (Ka) or affinity constant (K_{D}) of a FcRn antagonist or a component thereof (e.g., Fc domain or fragment thereof or albumin or fragment thereof) is determined.

Affinity measurements can be determined by standard methodology, for example, immunoassays, surface plasmon resonance (SPR; e.g., using BIAcore surface plasmon resonance (BIAcore, Inc., Piscataway, NJ) (Rich and Myszka Curr. Opin. Biotechnol 11: 54, 2000; Englebienne Analyst. 123:1599, 1998), isothermal titration calorimetry (ITC) or other kinetic interaction assays known in the art.

In some examples, the FcRn antagonist has a similar K_{D} or an improved K_{D} (i.e., a K_{D} value lower than) for FcRn than a human IgG1 Fc domain or a variant or fragment thereof.

Binding affinity for FcRn can also be determined non-quantitatively using flow cytometry. For example, CHO cells stably expressing the FcRn antagonist, or fragment thereof, are stained with alexa-488 labeled FcRn/β2m (to detect target binding) and anti-myc-alexa 647 (to detect expression) at acidic (pH 5.5) and neutral (pH 7.4) pH and analysed by flow cytometry. Relative binding to FcRn/β2m is determined, for example, by calculating mean fluorescence intensity relative to an unmodified human IgG1 Fc domain or a variant or fragment thereof.

### Determining Half-Life

FcRn antagonists encompassed by the present disclosure have an improved half-life, e.g., are modified to extend their half-life compared to a human IgG1 Fc domain or a variant or fragment thereof. Methods for determining half-life of a FcRn antagonist will be apparent to the skilled person.

For the purposes of clarification and as will be apparent to the skilled artisan based on the description herein, reference to “half-life” will be understood to refer to an increase in one or more of the following parameters: terminal half-life, mean residence time, area under the curve, decrease in volume of distribution and/or clearance rate. For example, extending the half-life of the FcRn antagonist means improving the plasma exposure of the antagonist or reducing the volume of distribution of the antagonist.

In one example, a FcRn antagonist of the disclosure has an improved alpha half-life, i.e., the rate of decline in plasma concentrations due to the process of redistribution.

The half-life of a FcRn antagonist of the disclosure can also be measured by pharmacokinetic studies, e.g., according to the method described by Kim et al, Eur J of Immunol 24:542, 1994. According to this method, protein is injected intravenously into mice and its plasma concentration is periodically measured as a function of time, for example at 3 minutes to 72 hours after the injection. The clearance curve thus obtained should be biphasic, that is, an alpha phase and beta phase. For the determination of the in vivo terminal half-life of the protein, the clearance rate in beta-phase is calculated and compared with that of a human IgG1 Fc domain or a variant or fragment thereof.

### In Vitro Cellular Assays

Various in vitro assays are available to assess the ability of a FcRn antagonist, to treat a disease or condition described herein.

In one example, the uptake and recycling of the FcRn antagonist is tested in an in vitro cellular assay.

Methods of assessing cellular uptake and recycling are known in the art and/or exemplified herein. For example, fluorescently labelled FcRn antagonist is incubated with cells expressing the human FcRn receptor on the cell surface. After addition of the labelled FcRn antagonist the progress of the protein recycling can be tracked and compared to a human IgG1 Fc domain or variant or fragment thereof by methods including flow cytometry and fluorescence microscopy (for example, confocal fluorescence microscopy). Changes to the normal recycling pathway for a particular albumin variant can be identified and characterised.

### Pharmacokinetic Analysis

In one example, the pharmacokinetic (PK) properties of the FcRn antagonist is assessed.

Methods of assessing the PK properties are known in the art and/or are exemplified herein. For example, FcRn antagonists are injected into transgenic mice expressing human FcRn receptor or other suitable mammalian hosts (e.g. rats, cynomolgus monkeys). In one example, the transgenic mice expressing human FcRn receptor are “hFcRn Tg32” homozygous mice (i.e., B6.Cg-Fcgrttm1Dcr Tg (FCGRT) 32Dcr/DcrJ; The Jackson Laboratory stock number 014565; or as described in Roopenian et al., J. Immunol 2003; 170:3528-3533). Plasma levels of FcRn antagonist are assessed using ELISA using commercially available methods.

### Immunoglobulin Clearance

In one example, the ability of a FcRn antagonist to reduce levels of circulating immunoglobulin is assessed.

For example, a known tracer antibody, e.g., IgG1 antibody, is administered to a subject, e.g., a mouse. The FcRn antagonist is then administered and the level of the antibody is determined at various time points. A FcRn antagonist induces more rapid reduction in the level of the tracer antibody than is observed in the absence of the antagonist.

In another example, the level of endogenous immunoglobulin, e.g., IgG is determined in the presence or absence of the antagonist.

### Pharmaceutical Compositions

Suitably, in compositions or methods for administration of the FcRn antagonist of the disclosure to a subject, the FcRn antagonist of the present disclosure (i.e., the albumin variant, or fragment thereof conjugated to a compound) is combined with a pharmaceutically acceptable carrier as is understood in the art. Accordingly, one example of the present disclosure provides a composition (e.g., a pharmaceutical composition) comprising the FcRn antagonist combined with a pharmaceutically acceptable carrier.

In general terms, by “carrier” is meant a solid or liquid filler, binder, diluent, encapsulating substance, emulsifier, wetting agent, solvent, suspending agent, coating or lubricant that may be safely administered to any subject, e.g., a human. Depending upon the particular route of administration, a variety of acceptable carriers, known in the art may be used, as for example described in Remington's Pharmaceutical Sciences (Mack Publishing Co. N.J. USA, 1991).

A FcRn antagonist of the present disclosure is useful for parenteral, topical, oral, or local administration, aerosol administration, or transdermal administration, for prophylactic or for therapeutic treatment. In one example, the FcRn antagonist is administered parenterally, such as subcutaneously or intravenously. For example, the FcRn antagonist is administered intravenously.

Formulation of a FcRn antagonist to be administered will vary according to the route of administration and formulation (e.g., solution, emulsion, capsule) selected. An appropriate pharmaceutical composition comprising a FcRn antagonist to be administered can be prepared in a physiologically acceptable carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. The FcRn antagonist can be stored in the liquid stage or can be lyophilized for storage and reconstituted in a suitable carrier prior to use according to art-known lyophilization and reconstitution techniques.

### Uses of FcRn Antagonists

The FcRn antagonist compositions of the present disclosure are particularly useful for reducing the serum levels of immunoglobulin or other Fc-containing agents (e.g., antibody-drug conjugates and immunoadhesins). Accordingly, in one example the disclosure provides a method of inhibiting FcRn function in a subject, the method generally comprising administering to the subject an effective amount of an FcRn antagonist or pharmaceutical composition of the disclosure.

The reduction of serum levels of immunoglobulin or Fc-containing agents is applicable to the treatment of antibody-mediated disorders (e.g. autoimmune diseases). Accordingly, in one example the instant disclosure provides methods of treating antibody-mediated disorders (e.g. autoimmune diseases) using the FcRn antagonist compositions disclosed herein.

Any antibody-mediated disorder can be treated using the FcRn antagonist compositions disclosed herein.

The FcRn antagonists of the instant disclosure are suited to treating antibody-mediated disorders characterized by an over production of serum immunoglobulin. Accordingly, in some examples, the FcRn antagonist compositions are used to treat hypergammaglobulinemia.

The FcRn antagonists can also be used in combination with one or more additional therapeutic agents. In some examples, the additional therapeutic agent is an anti-inflammatory agent. Any inflammatory agent can be used in combination with the FcRn antagonists disclosed herein. In some examples, the therapeutic agent is rituximab, daclizumab, basiliximab, muronomab-cd3, infliximab, adalimumab, omalizumab, efalizumab, natalizumab, tocilizumab, eculizumab, golimumab, canakinumab, ustekinumab, or belimumab. In examples, the additional therapeutic agent is leucocyte depleting agent (e.g., B-cell or T-cell depleting agent). Any leucocyte depleting agent can be used in combination with the FcRn antagonist disclosed herein. In some examples, the leucocyte depleting agent is a B-cell depleting agent. In some examples, the leucocyte depleting agent is an antibody against a cell surface marker. Suitable cell surface markers include, without limitation, CD10, CD19, CD20, CD21, CD22, CD23, CD24, CD37, CD53, CD70, CD72, CD74, CD75, CD77, CD79a, CD79b, CD80, CD81, CD82, CD83, CD84, CD85, or CD86. The FcRn antagonist and the additional therapeutic agent(s) can be administered to the subject simultaneously or sequentially, via the same or different route(s) of administration.

The FcRn antagonists of the instant disclosure are also suited to reducing the serum levels of an Fc-containing agent in subject. Such clearance is advantageous in cases where the Fc-containing agent is toxic (e.g., an antibody-drug conjugate or an agent that is immunogenic) because it reduces the exposure of the subject to the drug. Clearance is also advantageous in cases where the Fc-containing agent is an imaging agent that requires a low serum level of the agent to achieve better contrast imaging and/or minimise damage to normal tissue when the Fc-containing agent is radiolabelled. Accordingly, in some examples, the FcRn antagonists are used to reduce the serum levels of an Fc-containing agent in a subject that has been administered the Fc-containing agent. The serum levels of any Fc-containing agent (e.g., therapeutic or diagnostic agent) can be reduced using the FcRn antagonists disclosed herein. Non-limiting examples of Fc-containing agents include imaging agents (e.g., labeled antibodies), antibody drug conjugates, or immunogenic agents (e.g., non-human immunoglobulin or immunoadhesins). The FcRn antagonist can be administered simultaneously with the Fc-containing agent or sequentially (e.g., before or after the Fc-containing agent).

The FcRn antagonists disclosed herein can also be used in combination with the therapeutic protein to enhance the benefit of the therapeutic protein by reducing the levels of IgG, wherein IgG is responsible for the decreased bioavailability of a therapeutic protein.

In some examples the disclosure provides a method of reducing or preventing an immune response against a therapeutic compound.

One skilled in the art would be able, by routine experimentation, to determine what an effective, non-toxic amount of FcRn antagonist composition would be for the purpose of treating an antibody-mediated disorder. For example, a therapeutically active amount of a polypeptide may vary according to factors such as the disease stage (, age, sex, medical complications (e.g., immunosuppressed conditions or diseases) and weight of the subject, and the ability of the antibody to elicit a desired response in the subject. The dosage regimen may be adjusted to provide the optimum therapeutic response. For example, several divided doses may be administered daily, or the dose may be proportionally reduced as indicated by the exigencies of the therapeutic situation. Generally, however, an effective dosage is expected to be in the range of about 1 to 200 mg/kg body weight.

### Kits and Other Compositions of Matter

Another example of the disclosure provides kits containing a FcRn antagonist of the present disclosure useful for the reducing circulating autoantibodies in a subject in need thereof, the kit comprising:
(i) at least one FcRn antagonist or pharmaceutical composition or nucleic acid of the disclosure;
(ii) instructions for using the kit in reducing circulating autoantibodies in the subject; and
(iii) optionally, at least one additional therapy.

The disclosure also provides a kit for treating or preventing progression of an antibody-mediated disorder in a subject in need thereof, the kit comprising:
(i) at least one FcRn antagonist or pharmaceutical composition or nucleic acid of the disclosure;
(ii) instructions for using the kit in treating or preventing progression of an antibody-mediated disorder in the subject; and
(iii) optionally, at least one additional therapy.

In accordance with this example of the disclosure, the instructions (or package insert) is on or associated with the container. Suitable containers include, for example, bottles, vials, syringes, etc. The containers may be formed from a variety of materials such as glass or plastic. The label or package insert indicates that the FcRn antagonist is used for treating a subject eligible for treatment, e.g., one having or predisposed to developing a condition described herein, with specific guidance regarding dosing amounts and intervals of the FcRn antagonist and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

The kit optionally further comprises a container comprises a second medicament, wherein the FcRn antagonist is a first medicament, and which article further comprises instructions on the package insert for treating the subject with the second medicament, in an effective amount. The second medicament may be a therapeutic protein set forth above.

The present disclosure includes the following non-limiting Examples.

### Example 1: Materials and Methods

### Isolation of Bone Marrow Derived Macrophages (BMDMs)

Ten-week-old 32HOM huFcRnTg/Tg mice were euthanized by CO₂ asphyxiation and BMDMs were generated essentially as previously described (Lim et al., Biol Open. 2012 Sep. 15; 1(9):904-14, 2012). Monocytic cells were cryo-preserved at 6×10⁶ cells/mL for long term storage.

### Assessing Impact of FcRn Antagonists on IgG and Albumin Recycling in Macrophages Following Macropinocytosis.

Approximately 6×10⁶ BMDMs were seeded in 8 well removable chamber slides (Ibidi, Germany) at approximately 1.9×10⁵ cells/well. Cells were left to differentiate in 200 μL of BMDM media for 3 days before another 200 μL of BMDM media (RPMI, heat inactivated FCS (15%), 20% L cell media, 500 μL Pen/Strep; 2 mM Glutamax) was added to allow further differentiation of cells to macrophage-like cells. Cells were then starved 16 hours by removing BMDM media, washing twice with PBS and replacing with 200 μL C-RPMI (RPMI, heat inactivated FCS (10%), 500 μL Pen/Strep, 2 mM Glutamax) per well. Medium was then aspirated from wells and cells pre-incubated in the presence of DMSO (1:100 dilution) or protease inhibitor (1/100 dilution) for 4.5 h. Thirty minutes before the end of pre-incubation, mouse serum (at 1/100 dilution) was added to block surface Fc gamma receptors and incubated on ice for 30 minutes. Treatment samples including combinations of FcRn antagonists, HSA-AF488, IgG-AF568 and protease inhibitors were prepared and pre warmed at 37° C. Following incubation with mouse serum, cells were washed with PBS twice, pre-mixed pre-warmed experimental treatments were added and cells pulsed for 15 minutes, 37° C. (neutral pH). The cells were then washed twice in PBS, and either fixed immediately (=0 min chase) or C-RPMI+/−protease inhibitor added, and incubated for a 15 minute chase period, before washing and fixation.

Following 0 min and 15 minute chase time point, cells were fixed in 4% PFA for 10 mins, room temperature, washed once in PBS, 50 mM NH₄Cl to quench excess PFA, and incubated a further 10 mins, room temperature. They were then washed once more in PBS and blocked in 5% FCS/PBS for 30 mins Samples were then treated with Hoescht to stain for nuclei and the slides mounted in Mowiol mounting reagent.

Imaging was conducted by confocal microscopy. Fluorescence intensity of acquired images was analysed by the “analyse particle” plugin in Image J. Individual values were inputted into GraphPad prism and graphs were generated.

### Assessing FcRn Antagonist Affinity

FcRn antagonists tested in Tables 1, 2 and 3 (below) were captured using a surface-bound recombinant albumin binding protein (ABP) as described by Lejon et al (2004) Journal of Biological Chemistry, 279 (41), pp. 42924-42928. ABP (20 μg/mL in 10 mM Sodium acetate, pH 5) is directly immobilized onto the carboxymethyl dextran surface of CM5 sensorchips to approximately 2,000 RU using standard NHS/EDC chemistry at pH 5. Control G1Fc homodimer and full-length IgG1 were directly immobilized onto the carboxymethyl dextran surface of CM5 sensorchips to approximately 2,000 RU using standard NHS/EDC chemistry at pH 4.5.

FcRn antagonists (2 μg/mL) were prepared in running buffer at pH 7.3 and captured to approximately 350 RU in the active spot of each flow cell. The capture was equally stable under neutral and acidic conditions. No protein was captured onto the reference surface used for background subtraction. The immobilized ABP surface was prepared with ten cycles comprising of conditioning (60 second injection) with recombinant HSA (at 2 μg/mL) followed by 30 second regeneration with 6 M Guanidine-HCl. Sensorgrams were double subtracted using reference surface and blank buffer injection data obtained within each experiment. Data was fit to 1:1 Langmuir model with local Rmax and null refraction index (RI=0). Experiment running buffers (10 mM HEPES, 150 mM NaCl at pH 7.3 or pH 6.0) were prepared and filtered (0.22 μm) prior to use.

Purified soluble recombinant mammalian (i.e. human, cynomolgus monkey, mouse and/or rat) FcRn/β2m (~42 kDa) receptors were tested under neutral conditions (pH 7.3) at concentrations ranging from 39 nM to 20 μM and under acidic conditions (pH 6.0) at concentrations ranging from 7.8 nM to 2 μM. Analyte samples were prepared in 2-fold serial dilutions in running buffer. The association and dissociation rate constants of FcγRIIA(CD32), FcγRIIB(CD32), FcγRIIIA(CD16a) and FcRn/β2m receptors monitored for 120 and 240 seconds, respectively. The association and dissociation rate constants of FcγRI(CD64) were monitored for 240 and 600 seconds, respectively. The flow rate was set to 30 μL/min. Each ligand was tested in at least triplicate experiments.

In Table 2 (below), FcRn antagonists were tested under avidity conditions against biotinylated human FcRn/β2m tethered onto standard streptavidin or neutravidin sensorchips. Biotinylation of soluble recombinant human FcRn/β2m can be performed using either standard amine chemistry (e.g. Biotin-NHS) or enzymatic methods (eg. BirA biotin ligase) with comparable results. FcRn antagonist samples were prepared in running buffer in 2-fold dilutions ranging from 0.15 nM to 20 nM. The capture FcRn/β2m surface was prepared with 5 cycles comprising of conditioning (30 second injection) of recombinant IgG (100 nM, 15 μg/mL) followed by 30 second regeneration with 150 mM Tris, pH 8.

In Table 3 (below), purified soluble recombinant human FcY receptors (i.e. FcγRI(CD64), FcγRIIA(CD32), FcγRIIB(CD32), FcγRIIIA(CD16a), and FcRn/β2m were analysed under neutral conditions (pH 7.3) against FcRn antagonists. G1Fc homodimer and full-length IgG1 were used as baseline controls. Soluble analytes were prepared in 2-fold serial dilutions in running buffer. Human FcγRIIA(CD32), FcγRIIB(CD32), and FcγRIIIA(CD16a) receptors were tested at concentrations ranging from 39 nM to 50 μM while FcγRI(CD64) was tested at concentrations ranging from 0.098 to 100 nM.

Sensorgrams were double subtracted using reference surface and blank buffer injection data obtained within each experiment. Data was fit to 1:1 Langmuir model with local Rmax and null refraction index (RI=0). Experiment running buffers (10 mM HEPES, 150 mM NaCl at pH 7.3 or pH 6.0) were prepared and filtered (0.22 μm) prior to use.

FcRn antagonists demonstrate nanomolar affinity to mammalian FcRn/β2m when that Fc receptor is used as either an analyte (Table 1) or as a surface-bound ligand (Table 2). In Table 3, FcRn antagonists demonstrate binding affinities to soluble Fcy receptors that were comparable to the affinities observed for either a G1Fc homodimer or full-length IgG1.

### FcRn Antagonist Binding

FcRn/β2m protein complex (350-400 kDa, 1.28 mg/ml) was prepared in PBS and diluted in acetate buffer (pH 6.0) to 0.1 μM. Carbon-coated Cu grids (GSCU400CC, 400-mesh) were glow-discharged at 15 mA for 30 seconds. 4 μl of diluted protein sample was applied for 45 seconds, blotted off (Whatman No 1 filter paper) followed by 4 μl of H₂O, blotted off and finally negatively stained with 2% uranyl acetate for 30 seconds, blotted to remove the residual stain, and air-dried. Grids were imaged at room temperature using the TF30 (200 KeV) TEM at a magnification of ×39,000 with a pixel size of 2.79 Å. 32 images files were imported into cryoSPARC v 3.322 using the following input parameters: Pixel size (A) 2.79; Accelerating voltage (kV) 200 and spherical aberration (mm) 2.7. Patch CTF, followed by Blob picking (150-250 Å), Inspect particle picks and Extract particle picks (96 px box size) rendered 59879 particles for 2D classification. Selection of representative 2D class averages enabled template-based automatic particle picking (diameter 250 Å), which resulted in 39478 particles after an additional two rounds of 2D classification to further remove junk particles. Images from negative-staining experiments allow the identification of large globular domains (i.e. HSA) connected to long domains (i.e. Fc) and soluble domains (i.e. FcRn/β2m) loosely attached to Fc-like long domains, suggesting the presence of a stable FcRn/β2m/Antagonist complex in a 2:1 ratio.

### Example 2: Production of Extended Half-Life FcRn Antagonists

Fusion proteins were produced comprising recombinant human serum albumin (HSA) and either wild-type IgG1 Fc domain (huG1Fc) or a variant thereof comprising the following mutations M252Y, S254T, T256E, H433K and N434F (huG1FcYTEKF). Fusion proteins were produced with HSA linked to the N- or C-terminus of huG1Fc or huG1FcYTEKF.

huG1FcYTEKF was generated as benchmark molecule and was modelled on the FcRn antagonist Efgartigimod (Vyvgart), the only difference being five additional amino acids (EPKSC) at the N-terminus (SEQ ID NO:13). The internally generated molecule huG1FcYTEKF and the commercially available Vyvgart product were compared and were found to have comparable binding affinity (K_{D}) for the surface-bound huFcRn/b2m (i.e., huG1FcYTEKF; K_{D} 2.4 nM vs Vyvgart; K_{D} 2.3 nM at pH 7.3). In addition, the efficacy of internally generated benchmark huG1FcYTEKF in reducing IgG in non-human primate (NHP) studies was comparable to published data on Efgartigimod.

Homodimer and heterodimer variants of the FcRn antagonists were generated. For generation of homodimers comprising two albumins (e.g., Fc-albumin:Fc-albumin or albumin-Fc:albumin-Fc), cells were transfected with a single expression construct. (H464Q)

For generation of heterodimers, expression constructs encoding either an Fc-albumin or albumin-Fc molecule were co-transfected with an expression construct encoding an Fc sequence only at various ratios (e.g., 1:1, 1:0.5 or 1:0.25) to generate a certain amount of heterodimer FcRn antagonist (i.e., Fc-albumin:Fc or Albumin-Fc:Fc). The heterodimer products were separated from other expression products by protein A affinity chromatography and size exclusion chromatography. The heterodimers with huG1FcYPY and a single albumin molecule were also able to bind FcRn in vitro.

In initial SPR experiments, heterodimeric FcRn antagonist variants with a single albumin molecule fused to a dimeric huIgG1FcYPY (in each of the Fc-albumin and albumin-Fc orientations) bound to biotinylated human FcRn/β2m with similar affinity at pH 7.3 compared to the corresponding homodimeric FcRn antagonist variants comprising two albumins. Heterodimeric FcRn antagonists tested were: huG1FcYPY-huG1FcYPY-HSA (H464Q):huG1FcYPY; HSA-HSA:huG1FcYPY; huG1FcYPY:huG1FcYPY; and HSA (H464Q)-huG1FcYPY:huG1FcYPY.

Homodimeric FcRn antagonists were assessed for their ability to reduce levels of circulating IgG ‘tracer’ antibody (CSL360) in transgenic mice expressing human FcRn (huFcRn transgenic mice). The negative control group comprised mice that received no treatment and the positive control group comprised mice that received huG1FcYTEKF, a known FcRn antagonist. The FcRn antagonists were also assessed for their half-life.

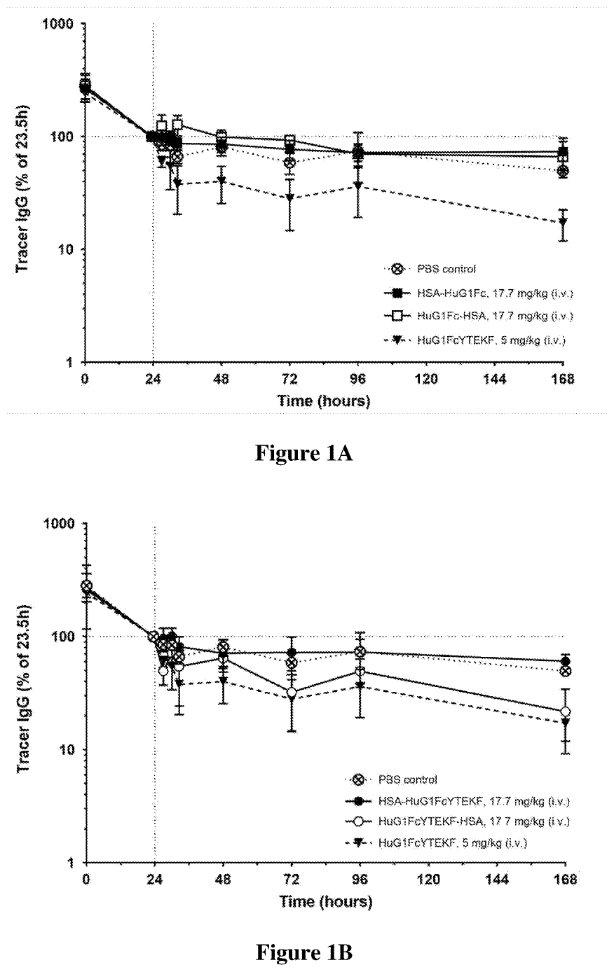

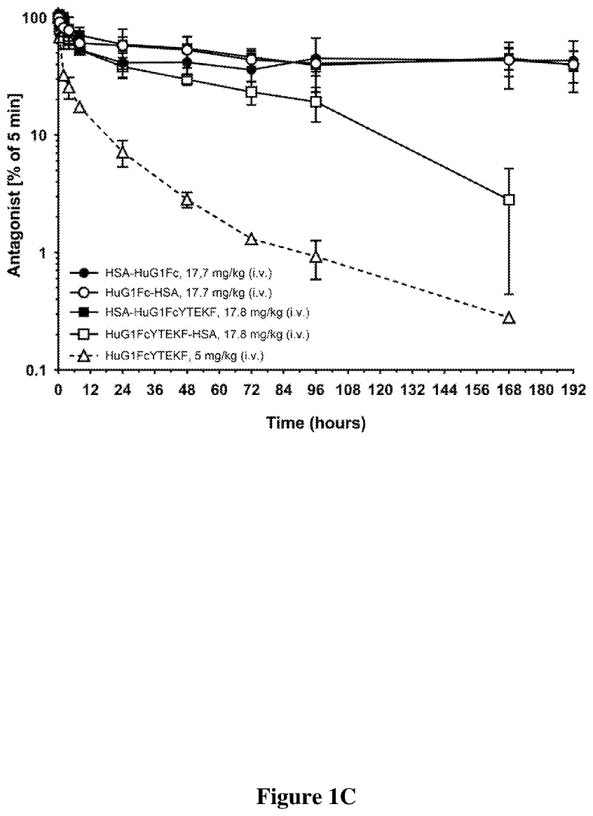

FIG. 1A shows that administration of C- and N-terminal HSA fusions with (wildtype) huG1Fc did not cause a reduction in circulating tracer antibody levels. FIG. 1B shows that huG1FcYTEKF-HSA (i.e. HSA fused to the C-terminus of huG1FcYTEKF; SEQ ID NO: 5) reduces the level of circulating IgG tracer antibody in vivo to a similar level as that observed with the huG1FcYTEKF positive control. However, the huG1FcYTEKF-HSA fusion protein had a substantially longer in vivo half-life than huG1Fc YTEKF (FIG. 1C). The same level of tracer antibody reduction was not observed when albumin was linked to the N-terminus of huG1FcYTEKF (FIG. 1B).

### Example 3: Production of Alternative Extended Half-Life FcRn Antagonists

M252Y, V308P, N434Y substitutions were introduced into the huG1 Fc domain (huG1FcYPY) and fusions to wild-type HSA and an HSA (H464Q) substitution variant were made. The HSA (H464Q) substitution variant is known to have reduced binding to FcRn.

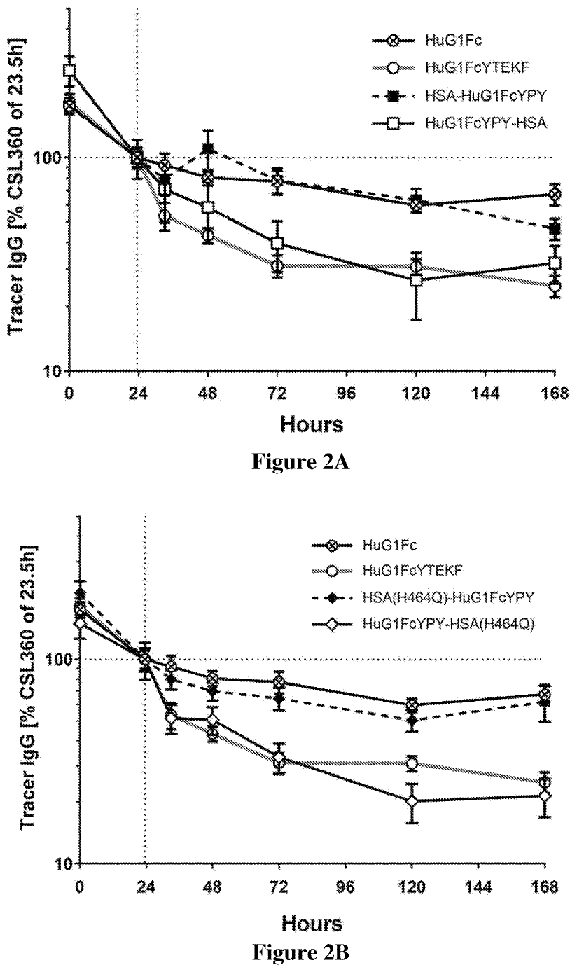

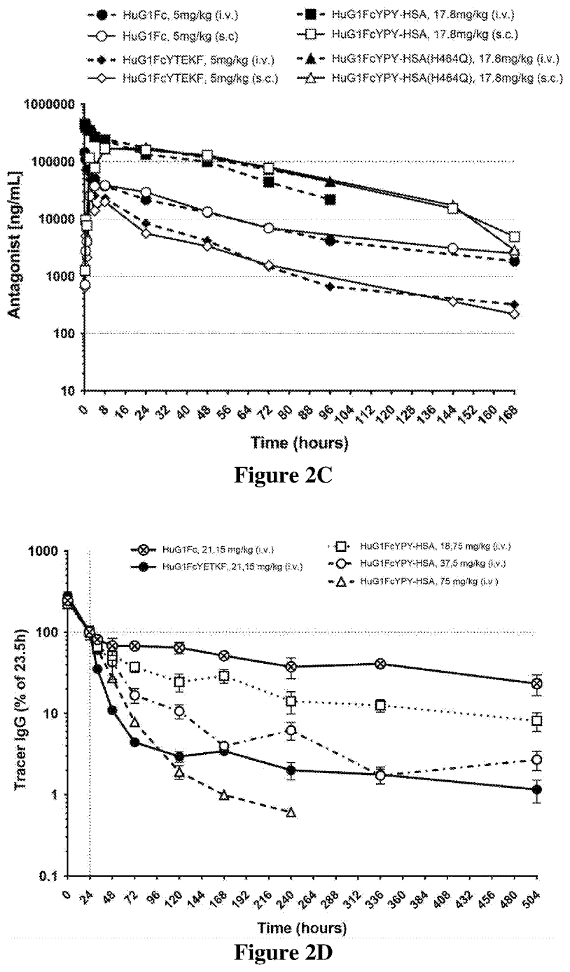

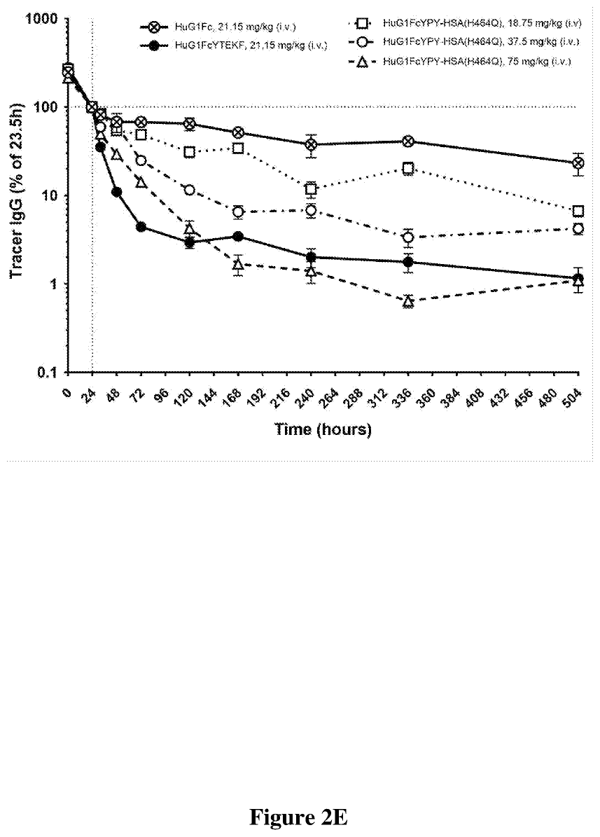

FIGS. 2A and B shows that huG1FcYPY-HSA and huG1FcYPY-HSA[H464Q) (SEQ ID NO: 11) reduced tracer antibody levels to a similar degree compared to huG1FcYTEKF. This result was observed irrespective of whether the HSA was wild-type or the H464Q substitution variant. Fusion of HSA to the N-terminus of huG1FcYPY was able to reduce circulating tracer antibody levels but not to the same degree as the C-terminal fusions (i.e., huG1FcYPY-HSA and huG1FcYPY-HSA (H464Q)).

FIG. 2C shows that similar pharmacokinetics were observed when FcRn antagonists of the disclosure were administered intravenously or subcutaneously, indicating that there is a high bioavailability of the FcRn antagonists following subcutaneous administration. Equimolar amounts of each FcRn antagonist were used.

FIG. 2D demonstrates that huG1FcYPY-HSA and huG1FcYPY-HSA (H464Q), respectively, reduce circulating levels of tracer antibody levels in a dose-dependent manner. These data suggest that the reduction in antibody levels is conferred by the Fc domain of the fusion protein and that the observed extended half-life conferred by HSA may be through a mechanism or mechanisms other than through binding to FcRn and FcRn-mediated recycling. These data were of particular interest to the inventors since binding of the HSA moiety of the FcRn antagonist to FcRn could potentially lead to reduced recycling of endogenous albumin, impacting upon albumin homeostasis, which has also been found to impact upon lipid homeostasis (Ward et al. Front Immunol. 13:892534, 2022).

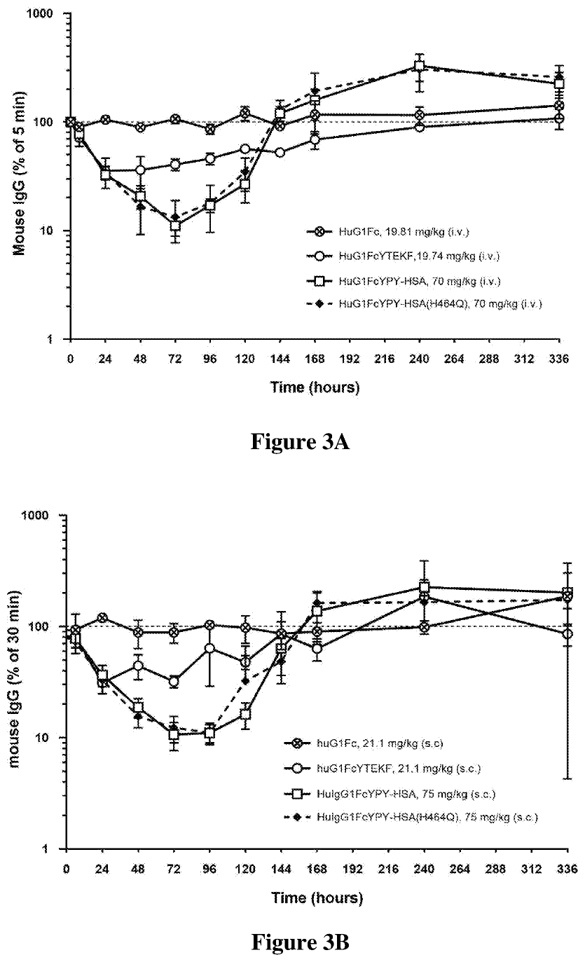

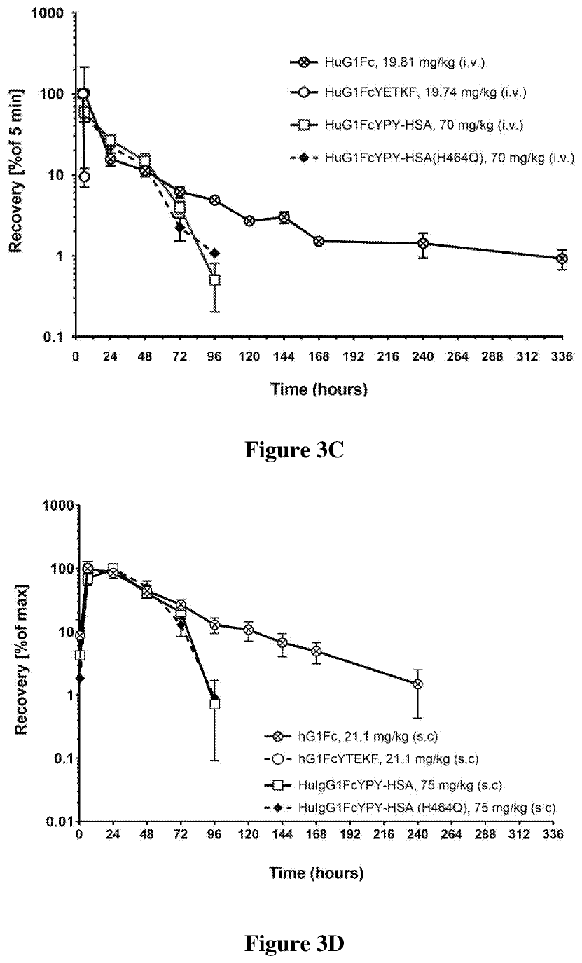

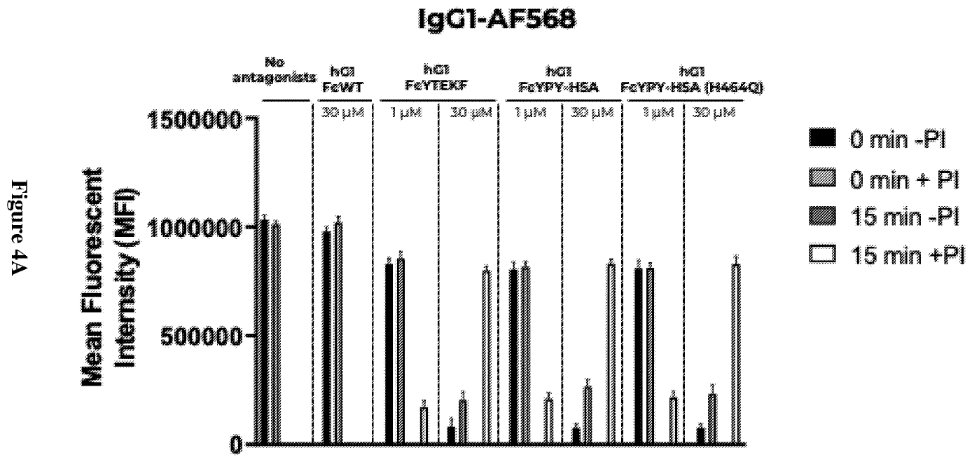

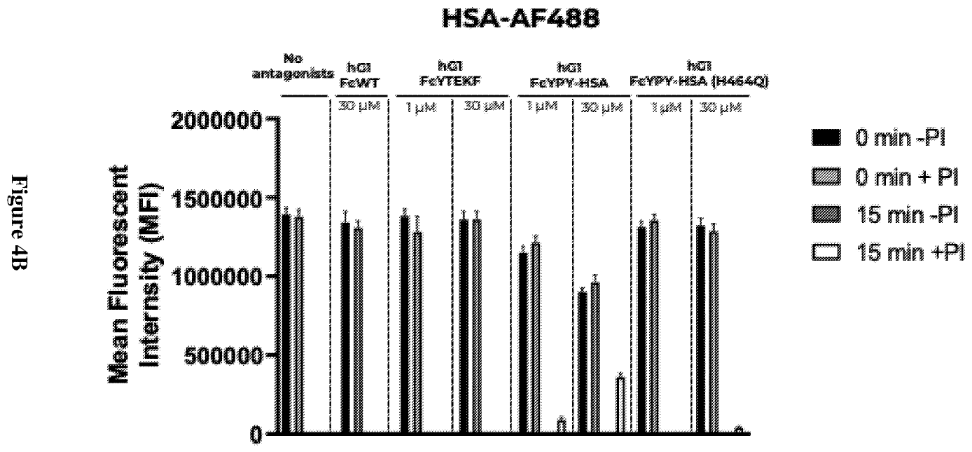

FIGS. 3A and B shows that a single dose of FcRn antagonists of the present disclosure reduced endogenous mouse IgG in wild-type mice to a level below that observed with huG1FcYTEKF (comparable to efgartigimod). This effect was observed irrespective of whether huG1FcYPY was fused to wild-type HSA or HSA (H464Q).

The FcRn antagonists of the present disclosure had substantially longer half-life compared to huG1FcYTEKF in wild-type mice as is shown in FIGS. 3C and D.

### Example 4: Affinity of FcRn Antagonists

Tables 1 and 2 relate to the affinity of binding of FcRn antagonists of the disclosure to FcRn as determined using SPR. The data shown in Table 1 were obtained using soluble human, cynomolgus monkey, mouse and rat FcRn complexed with β2m, and surface-bound FcRn antagonists of the disclosure at pH 6.0 and pH 7.3. The data in Table 2 relate to binding of soluble FcRn antagonists of the disclosure to surface-captured biotinylated human FcRn complexed with β2m (at pH 7.3). Table 3 relates to the binding of soluble FcY receptors to surface-captured FcRn antagonists.

TABLE 1

| | Human | Cyno | Mouse | Rat |
|---|---|---|---|---|
| Ligand (FcRn | FcRn/β2m | FcRn/β2m | FcRn/β2m | FcRn/β2m |
| antagonist) | K_{D} (nM)* | K_{D} (nM)* | K_{D} (nM)* | K_{D} (nM)* |
| huG1FcYPY-HSA | 881.4 ± 52.8 | 1509.9 ± 53.6 | 637.9 ± 12.7 | 4322.0 ± |
| (pH 7.3) (N = 4) | | | | 319.9 |
| huG1FcYPY-HSA | 88.1 ± 0.4 | 73.7 ± 2.2 | 180.8 ± 0.8 | 280.0 ± |
| (pH 6.0) (N = 4) | | | | 4.8 |
| huG1FcYPY-HSA | 813.6 ± 29.5 | 1535.7 ± 28.9 | 455.1 ± 30.3 | 4000.3 ± |
| (H464Q) (pH 7.3) | | | | 134.0 |
| (N = 4) | | | | |
| huG1FcYPY-HSA | 63.9 ± 0.4 | 55.6 ± 0.3 | 225.2 ± 0.9 | 354.9 ± |
| (H464Q) (pH 6.0) | | | | 10.5 |
| (N = 4) | | | | |

*Steady-state affinity values. Human and cyno FcRn/β2m interact with albumin and Fc domains of FcRn antagonists. Mouse and rat binding is limited to the Fc domain of FcRn antagonists.

TABLE 2

| | Human | Human | Human |
|---|---|---|---|
| | FcRn/β2m | FcRn/β2m | FcRn/β2m |
| Analyte | kₐ (1/Ms) | kₐ (1/s) | K_{D} (nM) |
| huG1FcYPY-HSA | 2.32E+05 | 3.50E−04 | 1.5 |
| huG1FcYPY-HSA(H464Q) | 2.56E+05 | 4.68E−04 | 1.8 |
| huG1FcYPY | 4.92E+05 | 5.32E−04 | 1.1 |
| huG1FcYTEKF | 9.70E+05 | 2.36E−03 | 2.4 |

TABLE 3

| | human | human | human | human |
|---|---|---|---|---|
| | FcyRIIIA | FcyRIIA | FcyRIIB | FcyRI |
| | (CD16a) | (CD32A) | (CD32B) | (CD64) |
| | K_{D} ± SEM | K_{D} ± SEM | K_{D} ± SEM | K_{D} ± SEM |
| Ligand | (μM) | (μM) | (μM) | (pM) |
| huG1FcYPY-HSA | 5.0 ± 0.42 | 2.2 ± 0.17 | 7.8 ± 0.30 | 134 ± 2.2 |
| | (N = 4) | (N = 4) | (N = 4) | (N = 3) |
| huG1FcYPY- | 4.2 ± 0.44 | 2.6 ± 0.23 | 8.0 ± 0.56 | 137 ± 3.5 |
| HSA(H464Q) | (N = 4) | (N = 4) | (N = 4) | (N = 3) |
| huG1Fc | 7.6 ± 0.02 | 1.5 ± 0.002 | 3.5 ± 0.02 | 86 ± 0.4 |
| | (N = 4) | (N = 2) | (N = 4) | (N = 4) |
| huIgG1 | 4.5 ± 0.1 | 1.9 ± 0.02 | 5.8 ± 0.34 | 345 ± 18.4 |
| | (N = 3) | (N = 3) | (N = 3) | (N = 3) |
| Privigen (polyclonal | 4.9 ± 0.04 | 2.1 ± 0.1 | 5.6 ± 0.04 | 94.7 ± 2.8 |
| IgG) | (N = 4) | (N = 2) | (N = 4) | (N = 4) |

TABLE 4

| | | Calculated | |
|---|---|---|---|
| | | Stoichiometry (S_{eq}) | |
| | | ${S}_{eq} = \frac{{Mw}_{L} \times {R}_{eq}}{{Mw}_{A} \times {R}_{L}}$ | |
| | FcRn binding | pH | |
| FcRn antagonist molecule | sites | 6.0 | pH 7.3 |
| HSA(H464Q) | 1 | 1 | No binding |
| huG1FcYPY-HSA(H464Q) (dimer) | 4 | 2 | 2 |
| huG1FcYPY-HSA (dimer) | 4 | 3 | 2 |
| huG1FcYPY-HSA(H464Q): | 3 | 2 | 2 |
| huG1FcYPY (heterodimer) | | | |
| huG1FcYPY-HSA:huG1FcYPY | 3 | 3 | 2 |
| (heterodimer) | | | |

### Example 5: FcRn Antagonists that do not Affect Albumin Recycling

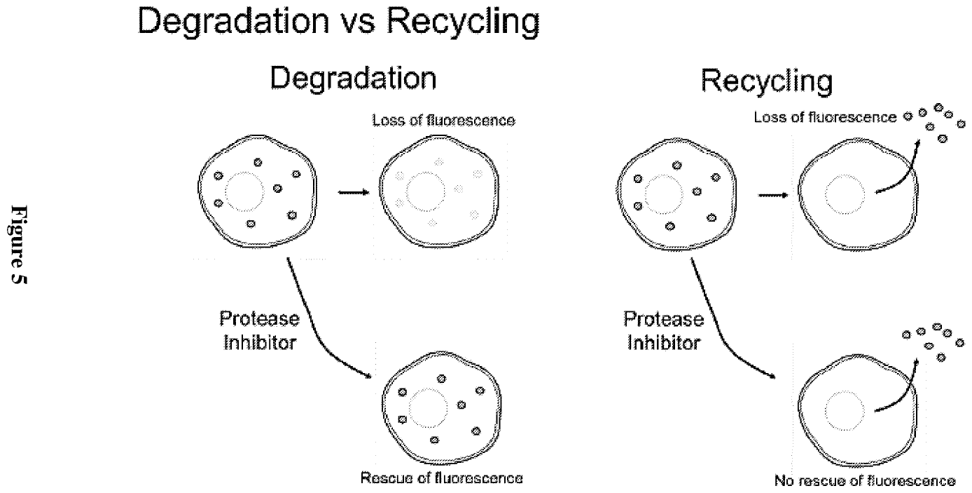

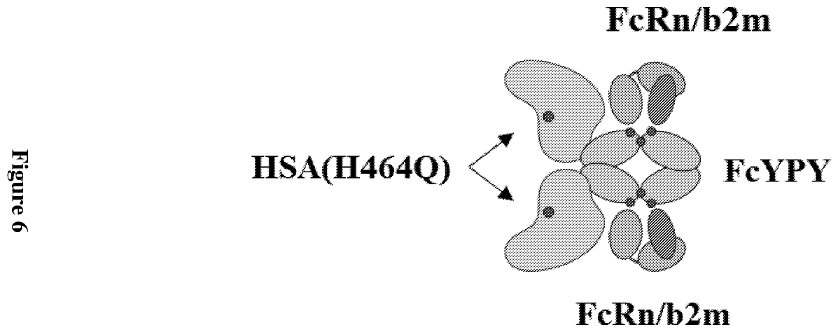

The ability of different FcRn antagonist molecules to antagonize IgG and albumin recycling was examined in BMDM from huFcRn transgenic mice (FIG. 4A and FIG. 4B). FIG. 5 shows a cartoon depicting how the assay described in the following section is assessed. Uptake of fluorescently labelled IgG (IgG1-AF568) by macropinocytosis was evident following a pulse of 10 minutes at 0-minute chase (FIG. 4A). After 15 minutes chase there was no longer detectable IgG1-AF568 and this signal was not rescued by protease inhibitors, suggesting IgG1-AF568 was recycled by the cell rather than transported to the lysosomal compartment for degradation. Similar results were observed in the presence of control protein, recombinant huG1Fc (the Fc domain of wild type human IgG1). It is understood that following fixation and permeabilization, membrane associated molecules within large macropinosomes are retained but free proteins within the macropinosome core are lost. Proteins that have trafficked to the later endosomal compartments, such as the lysosome, are more effectively retained/protected within the tighter membranous structures following the fixation/permeabilization steps (as shown in FIG. 5). A dose-dependent reduction of signal at time 0 post pulse in the presence of the FcRn antagonists huG1FcYTEKF, huG1FcYPY-HSA and huG1FcYPY-HSA (H464Q) suggests reduced interaction of IgG1-AF568 with membrane associated FcRn within the macropinosomes and loss of free IgG1-AF568 from the core of the macropinosome following the fixation/permeabilization step. Following a 15-minute chase prior to fixation/permeabilization, IgG1-AF568 was readily detected in the presence of protease inhibitors, indicating lysosomal rescue. A similar degree of dose-dependent FcRn antagonism of IgG1-AF568 recycling (corresponding to lysosomal degradation revealed by rescue in the presence of protease inhibitors at 15 minutes chase) was observed for huG1FcYTEKF, huG1FcYPY-HSA and huG1FcYPY-HSA (H464Q).

The impact of FcRn antagonists on albumin recycling (HSA-AF488) was also examined (FIG. 4B). In control samples, HSA-AF488 was taken up by macropinocytosis evident at 10 minutes pulse and 0-minute chase. However, by 15 minutes, there was no signal even in the presence of protease inhibitors, suggesting albumin was fully recycled. The antagonist huG1FcYTEKF, similarly did not impact on albumin recycling. The molecule huG1FcYPY-HSA, at 30 μM reduced HSA-AF488 signal observed at time 0 following pulse following fixation permeabilization (suggesting reduced interaction of HSA-AF488 with membrane FcRn and loss of free HSA-AF488 from macropinosome core). In addition, following 15 minutes chase prior to fixation/permeabilization, HSA-AF488 could be rescued from lysosomal degradation in the presence of protease inhibitors, suggesting that huG1FcYPY-HSA antagonizes albumin recycling. In contrast, there was minimal impact of the molecule, huG1FcYPY-HSA (H464Q) on albumin recycling with only a small amount of lysosomal rescue of HSA-AF488 observed with the highest concentration of huG1FcYPY-HSA (H464Q). In summary, these data demonstrate that the H464Q variant, has a reduced ability to antagonize albumin recycling in vitro, and therefore would be less likely to antagonize albumin recycling in vivo.

### Example 6: FcRn Antagonist Binding

Negative-staining experiments showed the conformation of a complex of one huG1FcYPY (H464Q) and two FcRn/β2m and suggested that the HSA (H464Q) moiety does not bind to FcRn/β2m. FIG. 6 shows a schematic representation of the putative complex.

### Example 7: FcRn Antagonist with Reduced Crosslinking Capacity

Fc-albumin fusion proteins (i.e., FcRn antagonists) have the potential to crosslink multiple FcRn receptors under acidic conditions (e.g. cellular endosomal compartment) by bridging neighbouring molecules via its Fc and albumin domains. The HSA (H464Q) mutant shows an approximate 10 μM affinity to human FcRn/β2m at acidic pH 6.0, compared to HSA (i.e. ~300 nM). The addition of the HSA (H464Q) mutation to the albumin in the FcRn antagonist reduces the affinity of the albumin to FcRn under acidic conditions (i.e. pH 6.0) limiting the FcRn/β2m interaction to the Fc domain of the FcRn antagonist. This is reflected by the reduction in the stoichiometry of HSA (H464Q) molecules to human FcRn/β2m (Table 5) as measured by SPR. Briefly, the stoichiometry (Seq) of each ligand is calculated at steady-state from the ratio of the maximum response at saturation (Rmax) to the amount of captured ligand (RL), multiplied by the ratio of the molecular weight of the ligand (MwL) over the analyte (MwA). This is a rearrangement of the equation for the expected theoretical Rmax. Capture levels (RL) between cycles are normalized by taking the binding levels at equilibrium (Req) and the capture levels (RL) for each cycle and carrying out the calculation according to Equation 1. This results in a table of Seq values for each concentration. These values can be plotted like a steady state fit to show the valency of binding at each concentration, and extrapolated to its maximum value to determine the stoichiometry of the ligand. The values are adjusted and fitted to a steady-state one-site model where the “Bmax” represents the stoichiometry of each interaction based on the capture and binding levels and molecular weights of the ligands and analyte. The molecular weights of the ligands and analytes are assumed to be 185 kDa for huG1FcYPY-HSA and huG1FcYPY-HSA (H464Q) fusions, 67 kDa for HSA, and 42 kDa for FcRn/β2m.

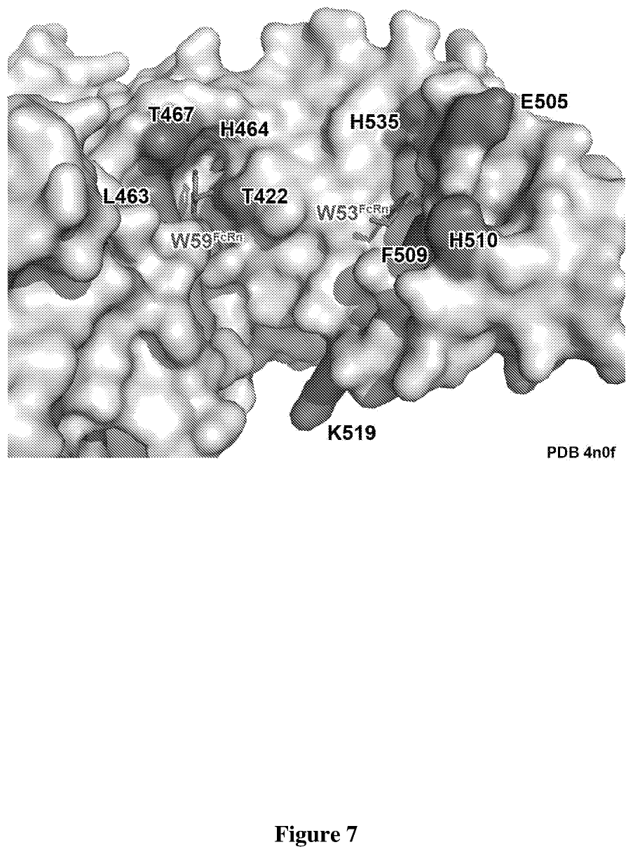

The role of His464 was demonstrated in the elucidation of the FcRn/β2m/HSA complex (Schmidt et al. (2013) Structure 21, 1966-1978). The model describes the role of His464, His510 and His535 stabilising the binding site for two conserved FcRn tryptophan residues (W53^{FcRn} and W59^{FcRn}) in a pH-dependent manner. Table 5 demonstrates that HSA residues surrounding the binding pockets for W53^{FcRn} and W59^{FcRn} (FIG. 7) can be mutated to drastically reduce the binding of HSA to FcRn/β2m compared to HSA (H464Q). Table 5 illustrates that the incorporation of additional albumin mutations into huG1FcYPY-HSA (H464Q) neutralises the albumin domain of the protein, resulting in FcRn/β2m binding affinities comparable to those observed for the G1FcYPY homodimer and to huG1FcYPY-HSA[25-384], where the HSA lacks the entire FcRn binding domain.

TABLE 5

| | HuFcRn/b2m | HuFcRn/b2m | HuFcRn/b2m | |
|---|---|---|---|---|
| Ligand | ka (1/Ms) | kd (1/s) | KD ± SEM (nM) | N |
| HSA | 1.82E+05 | 4.82E−02 | 265.2 ± 14.2 | 2 |
| HSA(H464Q) | 3.51E+04 | 3.74E−01 | 12307.1 ± 872.9 | 5 |
| G1FcYPY | 3.11E+05 | 5.25E−03 | 16.9 ± 0.2 | 4 |
| HSA[H464Q_T422W] | — | — | WB | 1 |
| HSA[H464Q_H535F] | — | — | WB | 1 |
| HSA[H464Q_T422M] | — | — | 762223.4 | 1 |
| HSA[H464Q_F509W] | — | — | 64343.3 | 1 |
| HSA[H464Q_K519E] | — | — | 36448.6 | 1 |
| HSA[H464Q_H510R] | — | — | 30901.9 | 1 |
| HSA[H464Q_E505K] | — | — | 25598.0 | 1 |
| HSA[H464Q_T467M] | — | — | 25309.3 | 1 |
| HSA[H464Q_L463W] | — | — | 20587.5 | 1 |
| HSA[H464Q_F509M] | — | — | 2144.8 | 1 |
| G1FcYPY-HSA[25-384]* | 1.39E+05 | 2.92E−03 | 21.1 | 1 |
| G1FcYPY- | 1.91E+05 | 2.39E−03 | 12.6 ± 0.3 | 4 |
| HSA[H464Q_H535F] | | | | |
| G1FcYPY- | 1.88E+05 | 2.47E−03 | 13.2 ± 0.1 | 4 |
| HSA[H464Q_T422W] | | | | |
| G1FcYPY- | 1.84E+05 | 2.95E−03 | 16.1 ± 0.2 | 4 |
| HSA[H464Q_T422W_H535F] | | | | |

*HSA lacking C-terminal DIII (i.e. FcRn binding domain).

### Example 8: Impact of FcRn Antagonists on Endogenous Cynomolgus Monkey IgG, IgA and IgM Following a Single Intravenous Dose

Each compound (i.e., huG1FcYPY-HSA, huG1FcYPY-HSA (H464Q) and huG1FcYTEKF) was administered as a single bolus intravenous (I.V.) injection of FcRn antagonist dosed at equimolar doses in 3 male animals of approximately 4 kg each. Briefly, FcRn antagonists were dosed at equimolar levels of 75 mg/kg for huG1FcYPY-HSA and huG1FcYPY-HSA (H464Q) and 21.15 mg/kg for huG1FcYTEKF. No negative clinical signs, abnormal hematology, blood biochemistry or cytokine profile deviations were observed following treatment. Albumin and lipids were also largely unaffected.

Blood was sampled at pre-dose, 0.0833 h, 0.5 h, 3 h, 8 h, 24 h, 48 h, 72 h, 168 h, Day 8, Day 12, Day 15, Day 22, Day 29, Day 36, Day 43, Day 50, Day 57, Day 64, Day 71 and Day 85. The low limit of quantitation (LLOQ) for tested compounds was reported with 1000 ng/ml for the huG1FcYPY-HSA and huG1FcYPY-HSA (H464Q) and 250 ng/ml for huG1FcYTEKF.

Endogenous Ig analysis was completed using a qualified enzyme-linked immunosorbent assay (ELISA) method developed with the Isotyping Panel 1 Human/NHP Kit, Cat. No K15203D.

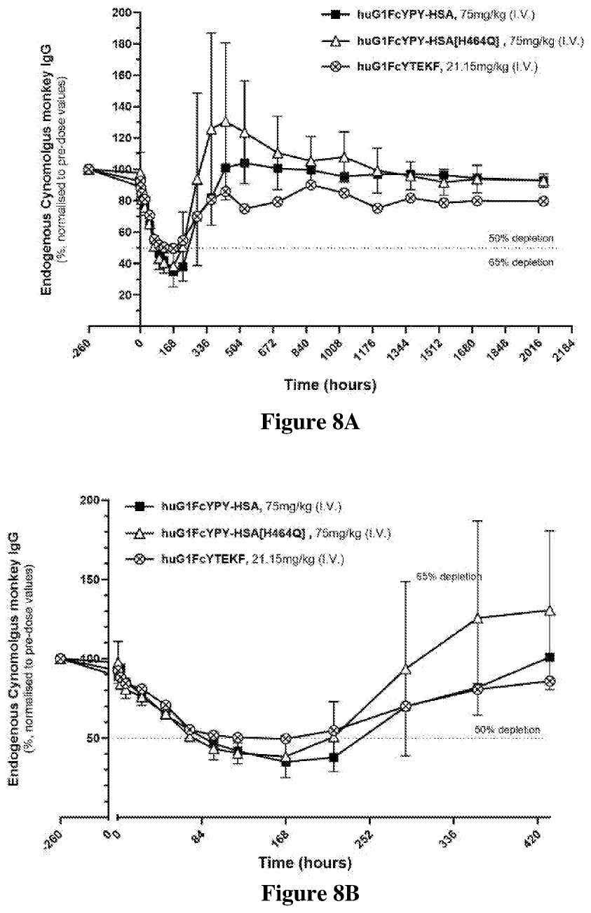

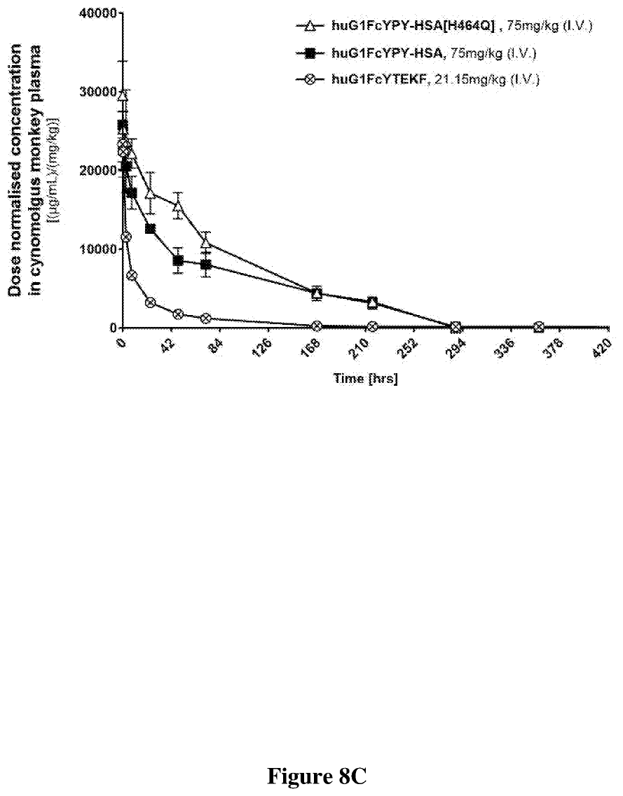

IgG levels in all animals followed a consistent trend with a progressive reduction up until 168 hours (7 days) post-dose (FIGS. 8A and 8B). HuG1FcYPY-HSA (H464Q) was most efficacious in reducing IgG levels, by up to 75%, with up to 68% IgG reduction in animals treated with huG1FcYPY-HSA, and up to 59% IgG reduction in animals treated with huG1FcYTEKF. Endogenous IgG returned to predose levels in all groups by Day 57.

Levels of IgA generally remained around predose levels in all animals at all time points (not shown).

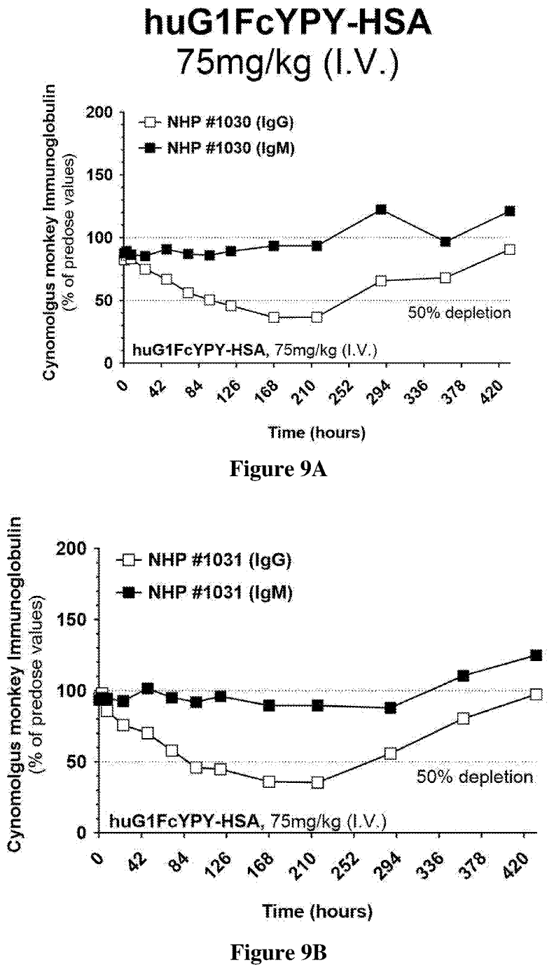

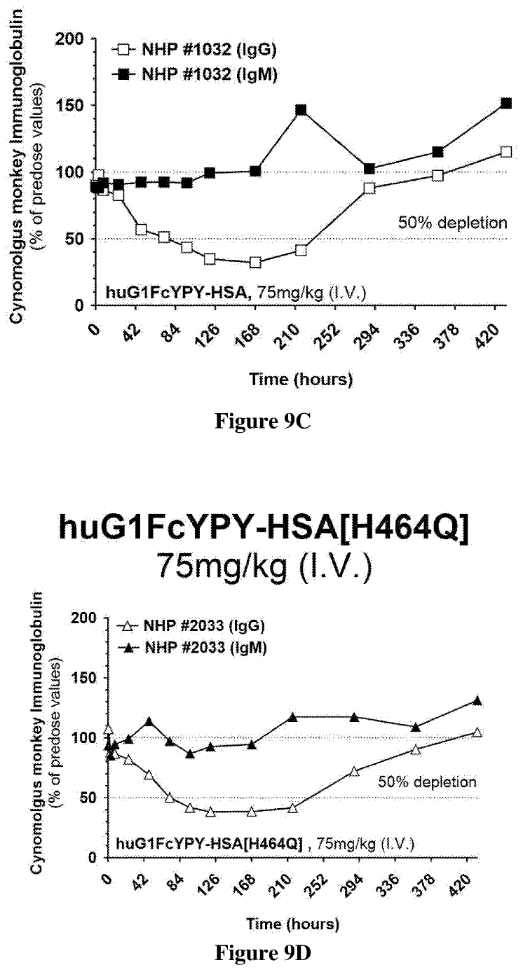

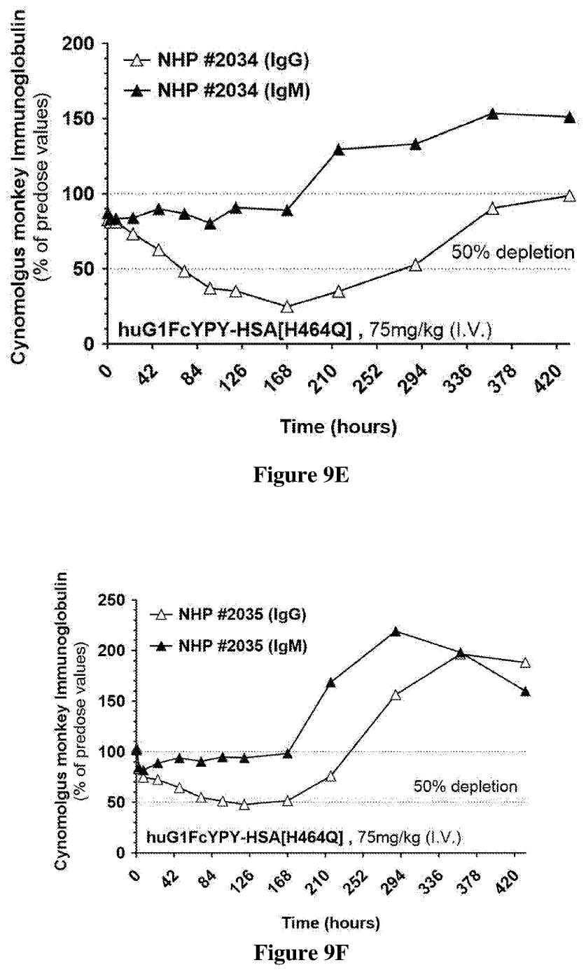

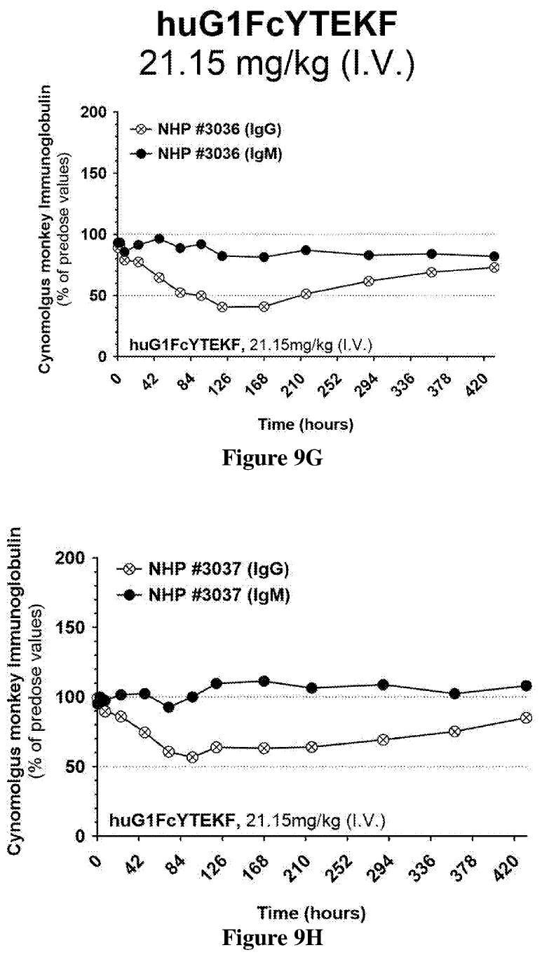

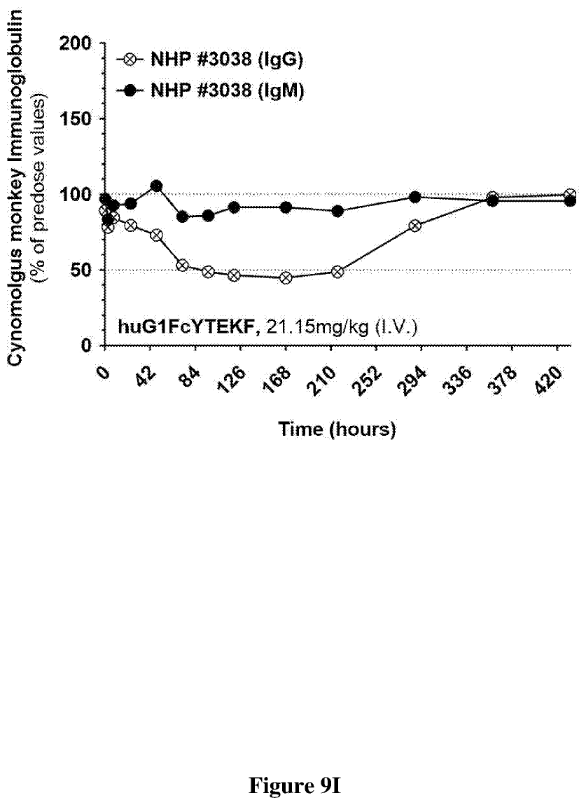

IgM levels generally remained consistent in all animals up until 168 hours post-dose after which an elevation in IgM was observed in animals treated with huG1FcYPY-HSA (H464Q) and huG1FcYPY-HSA molecules (FIG. 9). No clear trends in IgM levels were seen in huG1FcYTEKF-treated animals. The elevation in IgM is suggestive of anti-drug antibody (ADA) generation. An ADA response in NHP to human proteins is not predictive of ADA in humans but may result in an underestimate of the PK and PD of the drug. Indeed, the strong rebound in IgG, preceded by or correlating with a spike in IgM in some animals may have been partly driven by ADA (FIG. 8B and FIG. 9).

### Example 9. Pharmacokinetics of FcRn Antagonists in Cynomolgus Monkey Serum

Pharmacokinetics analysis of FcRn antagonists followed the specific quantitation and bioanalytical evaluation of huG1FcYPY-HSA, huG1FcYPY-HSA (H464Q) and huG1FcYTEKF from cynomolgus monkey serum samples after single intravenous dose administration. Quantitation was performed using a qualified enzyme-linked immunosorbent assay (ELISA) method. Calibration standards, ranging from 75,000 to 1,000 ng/mL, were run in duplicate wells, and the mean signal was regressed against the nominal concentrations using a 4PL curve fit with 1/y weighting. Calibration standards were prepared by spiking blank pooled cynomolgus monkey serum with appropriate amounts of huG1FcYPY-HSA, huG1FcYPY-HSA (H464Q) and huG1Fc YTEKF stock solutions. The calibration standards were prepared fresh before each experiment and discarded after use.

Briefly, FcRn antagonists were quantified from blood sampled at pre-dose, 0.0833 h, 0.5 h, 3 h, 8 h, 24 h, 48 h, 72 h, 168 h, Day 8, Day 12, Day 15, Day 22, Day 29, Day 36, Day 43, Day 50, Day 57, Day 64, Day 71 and Day 85. The measured concentrations of at least 75% of the calibration standards were within +20% of their theoretical concentrations (within +25% at the upper and lower quantitation limits). Additionally, the coefficient of variation of the duplicate wells for each standard had to be ≤20%.

A non-compartmental analysis performed to compare the PK profiles profiles for huG1FcYPY-HSA and demonstrates distinct time-concentration huG1FcYPY-HSA (H464Q) compared to huG1FcYTEKF (FIG. 8C and Table 6). Compounds huG1FcYPY-HSA and huG1FcYPY-HSA (H464Q) showed two phases of distribution (a and B phase). The terminal phase of these molecules (after Day 8) is governed by a steep terminal slope, possibly due to ADA suggested by an increase in IgM in some animals and strong IgG rebound as described above. Contribution of Target Mediated Drug Disposition (TMDD), however, cannot be excluded. Drug product was no longer detected at 294 h for huG1FcYPY-HSA (H464Q) and only in one animal for huG1FcYPY-HSA.

The pharmacokinetics of huG1FcYTEKF was characterized by three phases: a steep initial slope (α phase) followed by an intermediate slope (β phase) and a rather shallow slope during the terminal phase. huG1FcYTEKF showed a significantly steeper slope in the initial phase, suggesting a higher total volume of distribution compared to huG1FcYPY-HSA and huG1FcYPY-HSA (H464Q) compounds, which seemed to be more confined to the plasma space. The dose-normalized area under the curve (AUC) (as (DN AUC last (ug/mL*h/mg)) was significantly larger for huG1FcYPY-HSA (H464Q), compared to huG1FcYPY-HSA and huG1FcYTEKF.

The reduced volume of distribution for both albumin fusions compared to benchmark molecule may be due to a combination of both reduced tissue penetrance and reduced kidney clearance. Of interest the volume of distribution for the huG1FcYPY-HSA (H464Q) molecule was less than that of the WT fusion protein. In addition to its role in recycling of albumin and IgG, FcRn has been found to have a role in tissue distribution of albumin (Feng et al, Mol Pharm. 2019 Jun. 3; 16(6): 2385-2393.). One possibility is that, while on balance, albumin fusion reduces the volume of distribution for both molecules, the impaired interaction of FcRn with albumin domains within huG1FcYPY-HSA (H464Q) may further limit tissue distribution of this molecule due to reduced FcRn mediated transcytosis. An alternative possibility is that the huG1FcYPY-HSA, due to potential for tetrameric interaction with FcRn (via two Fc and two albumin domain binding sites), is more strongly sequestered within target cells, with cross linking of FcRn that may in turn lead to enhanced lysosomal degradation of the FcRn/drug complex as previously described with FcRn crosslinking (Weflen et al 2013. Multivalent immune complexes divert FcRn to lysosomes by exclusion from recycling sorting tubules (Mol Biol Cell 2013)

Since hematopoietic cells and endothelial cells are responsible for the majority of IgG recycling, increased retention of an FcRn antagonist within plasma with access to these relevant cell types, as has been achieved for the huF1FcYPY-HSA (H464Q) molecule is likely to be of benefit.

TABLE 6
Key non-compartmental analysis parameters of pharmacokinetics (PK) of
huG1FcYPY-HSA, huG1FcYPY-HSA(H464Q) and huG1FcYTEKF
detected in non-human primate (cynomolgus monkeys) plasma
following a single intravenous injection of antagonist.

| | huG1FcYPY- | huG1FcYPY- | huG1FcYTEKF |
|---|---|---|---|
| | HSA(H464Q) | HSA | 21.115 mg/kg |
| | 75 mg/kg (I.V.) | 75 mg/kg (I.V.) | (I.V.) |
| Dose-normalized Cmax | 8.13 | 6.38 | 7.76 |
| DN Cmax (ug/mL/mg) | | | |
| Dose-normalized AUC | 559 | 389 | 110 |
| last DN AUC last (ug/ | | | |
| mL*h/mg) | | | |
| Half-life of the β phase | 67.3 | 115 | 42.1 |
| (Distribution phase, | | | |
| 2-8 days) t_{1/2}(β) (h) | | | |
| Terminal half-life | 9.64 | 11.3 | 72.1 |
| (elimination phase, >8 | | | |
| days) t_{1/2} (h) | | | |
| Mean residence time | 69.2 | 76.6 | 70.2 |
| MRT (h) | | | |
| Initial volume of | 123 | 157 | 129 |
| distribution V_{c} (mL) | | | |
| Volume of distribution | 124 | 197 | 596 |
| V_{SS} (mL) | | | |
| Apparent terminal volume | 25 | 42 | 883 |
| of distribution | | | |
| V_{Z/F} (mL) | | | |

### Example 10. FcRn Antagonists on Cynomolgus Monkey IgG, IgA and IgM Following a Single Subcutaneous Dose

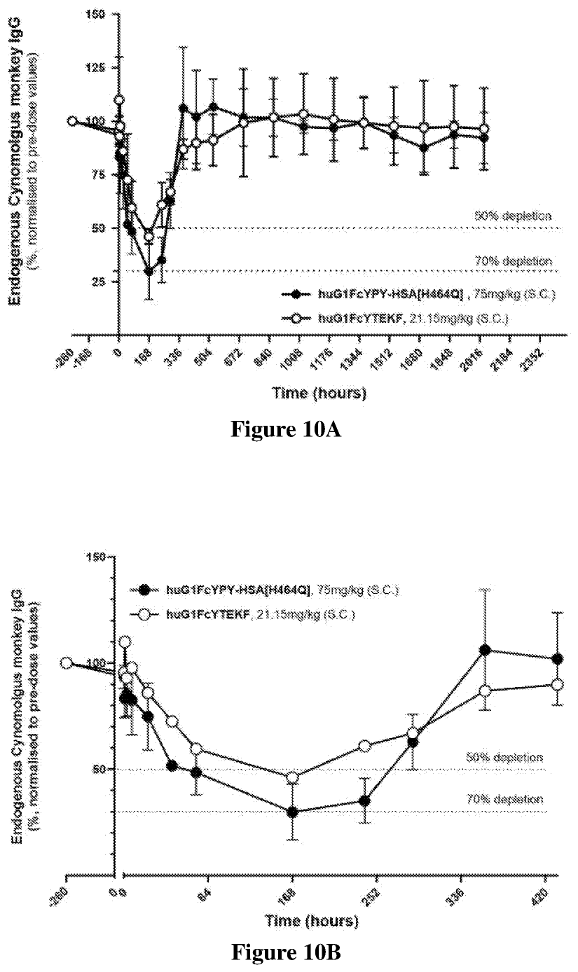

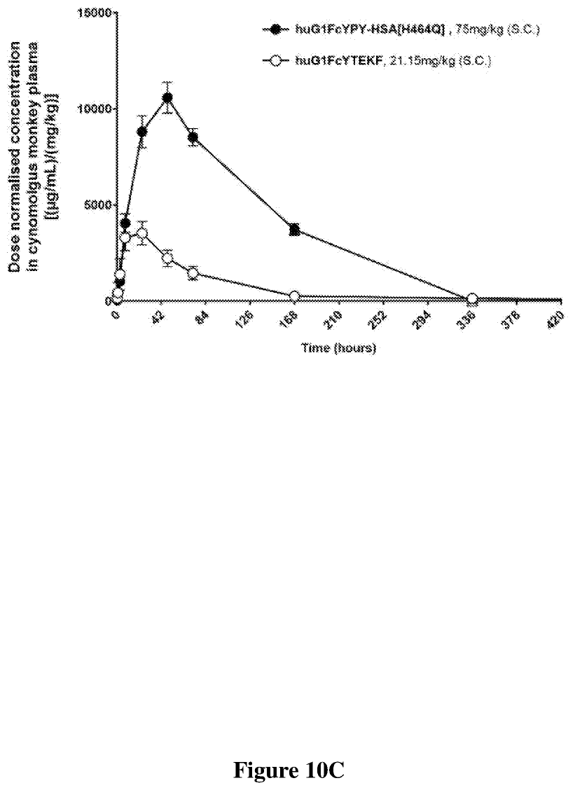

Each compound (i.e., huG1FcYPY-HAS, huG1FcYPY-HSA (H464Q) and huG1FcYTEKF) was administered as a single bolus subcutaneous (S.C.) injection of FcRn antagonist in 3 male animals of approximately 4 kg each (FIGS. 10A and 10B). Two FcRn antagonists, huG1FcYPY-HSA (H464Q) and huG1FcYTEKF were dosed at equimolar levels of 75 mg/kg and 21.15 mg/kg, respectively. These molecules were well tolerated at the concentrations tested. No negative clinical signs, abnormal hematology, blood biochemistry or cytokine profile deviations were observed.

Blood was sampled at pre-dose, 0.0833 h, 0.5 h, 3 h, 8 h, 24 h, 48 h, 72 h, 168 h, Day 8, Day 12, Day 15, Day 22, Day 29, Day 36, Day 43, Day 50, Day 57, Day 64, Day 71 and Day 85. The low limit of quantitation (LLOQ) for tested compounds was reported with 1000 ng/mL for huG1FcYPY-HSA (H464Q) and 250 ng/ml for huG1FcYTEKF. Endogenous Ig analysis was completed using a qualified enzyme-linked immunosorbent assay (ELISA) method developed with the Isotyping Panel 1 Human/NHP Kit, Cat. No K15203D.

Animals treated with huG1FcYPY-HSA (H464Q) and huG1FcYTEKF displayed a progressive reduction in IgG levels until 168 h (Day 7) (FIGS. 10A and 10B). As in IV studies, huG1FcYPY-HSA (H464Q) was more effective in driving IgG depletion (up to 80% IgG depletion) than the benchmark huG1FcYTEKF (up to 60% depletion). Endogenous IgG returned to their predose levels in both groups by Day 18.

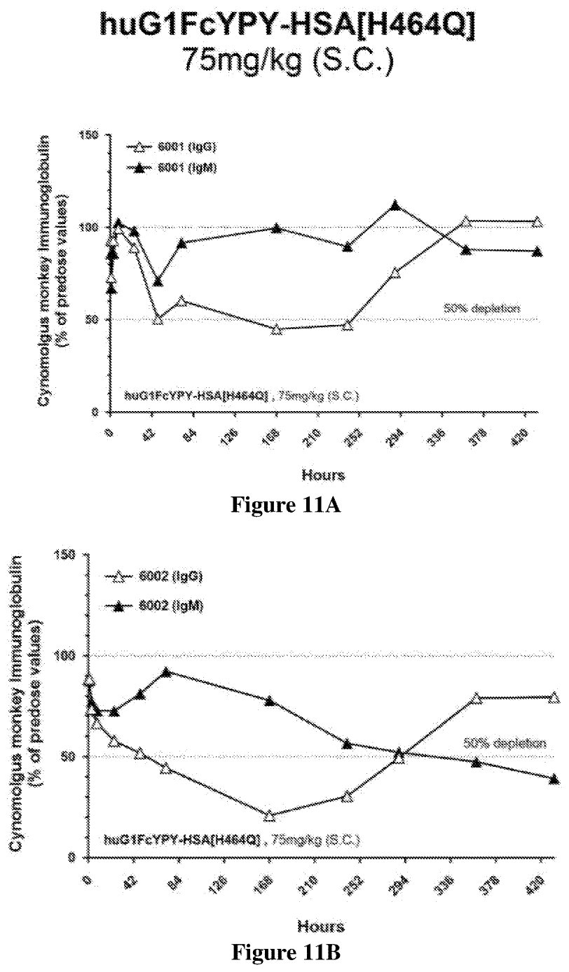

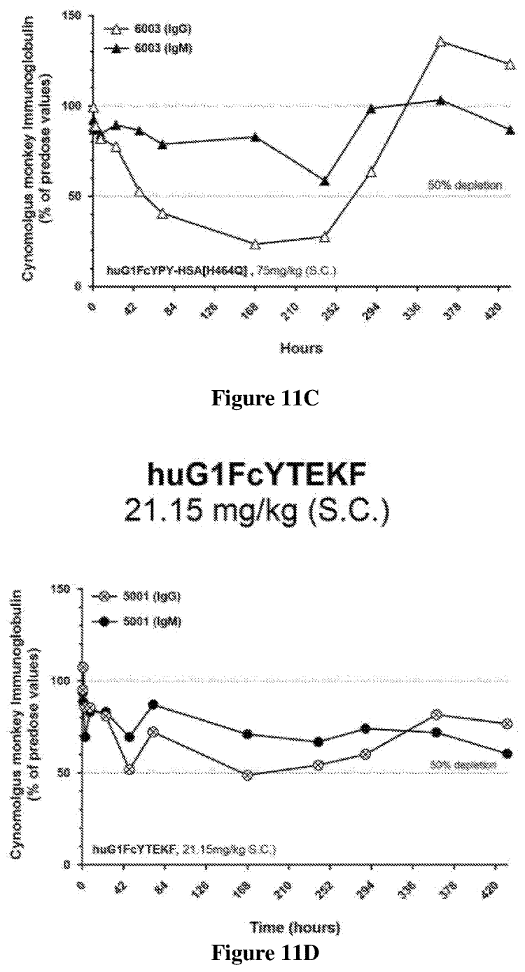

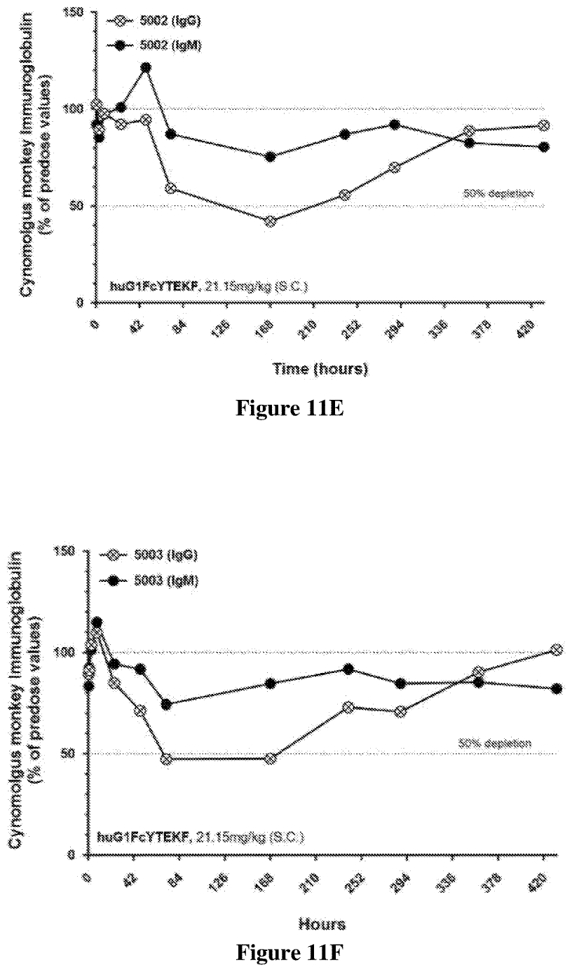

IgM levels generally remained consistent in all animals up until 168 hours post-dose after which an elevation in IgM was observed in animals treated with huG1FcYPY-HSA (H464Q) (FIG. 11). No clear trends in IgM levels were seen in huG1FcYTEKF-treated animals.

There was little evidence for drug related effects on IgM or IgA levels following subcutaneous administration of FcRn antagonists (not shown).

TABLE 7
Key non-compartmental analysis parameters of pharmacokinetics (PK) of
huG1FcYPY-HSA(H464Q) and huG1FcYTEKF detected in non-human primate
(cynomolgus monkeys) plasma following a single subcutaneous injection of antagonist.
(*) The calculated t_{1/2} (h) (terminal) phase values are limited by the non-linear terminal
clearance rate of huG1FcYPY-HSA(H464Q).

| | huG1FcYPY-HSA(H464Q) | huG1FcYTEKF |
|---|---|---|
| | (75 mg/kg) | (21.15 mg/kg) |
| Dose-normalized Cmax | 3.41 | 1.22 |
| DN Cmax (ug/mL/mg) | | |
| Dose-normalized AUC last | 397 | 91.8 |
| DN AUC last (ug/mL*h/mg) | | |
| Time of Cmax | 48.0 | 16.7 |
| tₘₐₓ (h) | | |
| Half-life of the β phase | 79.8 | — |
| (Distribution phase, (tₘₐₓ to Day 8)) | | |
| t_{1/2}(β) (h) | | |
| Terminal half-life | 24.5* | 64.5 |
| (elimination phase >8 days) | | |
| t_{1/2} (h) | | |
| Apparent terminal volume of | 88.8* | 1010 |
| distribution | | |
| V_{Z}/F (mL) | | |

Table of sequences

| SEQ ID | | |
|---|---|---|
| NO: | Description | Sequence |
| 1 | Human serum | DAHKSEVAHRFKDLGEENFKALVLIAFAQYLQQCPFEDHVKLVN |
| | albumin (HSA) | EVTEFAKTCVADESAENCDKSLHTLFGDKLCTVATLRETYGEMA |
| | | DCCAKQEPERNECFLQHKDDNPNLPRLVRPEVDVMCTAFHDNEE |
| | | TFLKKYLYEIARRHPYFYAPELLFFAKRYKAAFTECCQAADKAA |
| | | CLLPKLDELRDEGKASSAKQRLKCASLQKFGERAFKAWAVARLS |
| | | QRFPKAEFAEVSKLVTDLTKVHTECCHGDLLECADDRADLAKYI |
| | | CENQDSISSKLKECCEKPLLEKSHCIAEVENDEMPADLPSLAAD |
| | | FVESKDVCKNYAEAKDVFLGMFLYEYARRHPDYSVVLLLRLAKT |
| | | YETTLEKCCAAADPHECYAKVFDEFKPLVEEPQNLIKQNCELFE |
| | | QLGEYKFQNALLVRYTKKVPQVSTPTLVEVSRNLGKVGSKCCKH |
| | | PEAKRMPCAEDYLSVVLNQLCVLHEKTPVSDRVTKCCTESLVNR |
| | | RPCFSALEVDETYVPKEFNAETFTFHADICTLSEKERQIKKQTA |
| | | LVELVKHKPKATKEQLKAVMDDFAAFVEKCCKADDKETCFAEEG |
| | | KKLVAASQAALGL |
| 2 | Human IgG1 Fc | EPKSCDKTHTCPPCPAPELLGGPSVFLFPPKPKDTLMISRTPEV |
| | (huG1Fc) | TCVVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNY |
| | | KTTPPVLDSDGSFFLYSKLTVDKSRWQQGNVFSCSVMHEALHNH |
| | | YTQKSLSLSPGK |
| 3 | huG1Fc-HSA (FcRn | EPKSCDKTHTCPPCPAPELLGGPSVFLFPPKPKDTLMISRTPEV |
| | antagonist) | TCVVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNY |
| | | KTTPPVLDSDGSFFLYSKLTVDKSRWQQGNVFSCSVMHEALHNH |
| | | YTQKSLSLSPGKDAHKSEVAHRFKDLGEENFKALVLIAFAQYLQ |
| | | QCPFEDHVKLVNEVTEFAKTCVADESAENCDKSLHTLFGDKLCT |
| | | VATLRETYGEMADCCAKQEPERNECFLQHKDDNPNLPRLVRPEV |
| | | DVMCTAFHDNEETFLKKYLYEIARRHPYFYAPELLFFAKRYKAA |
| | | FTECCQAADKAACLLPKLDELRDEGKASSAKQRLKCASLQKFGE |
| | | RAFKAWAVARLSQRFPKAEFAEVSKLVTDLTKVHTECCHGDLLE |
| | | CADDRADLAKYICENQDSISSKLKECCEKPLLEKSHCIAEVEND |
| | | EMPADLPSLAADFVESKDVCKNYAEAKDVFLGMFLYEYARRHPD |
| | | YSVVLLLRLAKTYETTLEKCCAAADPHECYAKVFDEFKPLVEEP |
| | | QNLIKQNCELFEQLGEYKFQNALLVRYTKKVPQVSTPTLVEVSR |
| | | NLGKVGSKCCKHPEAKRMPCAEDYLSVVLNQLCVLHEKTPVSDR |
| | | VTKCCTESLVNRRPCFSALEVDETYVPKEFNAETFTFHADICTL |
| | | SEKERQIKKQTALVELVKHKPKATKEQLKAVMDDFAAFVEKCCK |
| | | ADDKETCFAEEGKKLVAASQAALGL |
| 4 | HSA-huG1Fc | DAHKSEVAHRFKDLGEENFKALVLIAFAQYLQQCPFEDHVKLVN |
| | | EVTEFAKTCVADESAENCDKSLHTLFGDKLCTVATLRETYGEMA |
| | | DCCAKQEPERNECFLQHKDDNPNLPRLVRPEVDVMCTAFHDNEE |
| | | TFLKKYLYEIARRHPYFYAPELLFFAKRYKAAFTECCQAADKAA |
| | | CLLPKLDELRDEGKASSAKQRLKCASLQKFGERAFKAWAVARLS |
| | | QRFPKAEFAEVSKLVTDLTKVHTECCHGDLLECADDRADLAKYI |
| | | CENQDSISSKLKECCEKPLLEKSHCIAEVENDEMPADLPSLAAD |
| | | FVESKDVCKNYAEAKDVFLGMFLYEYARRHPDYSVVLLLRLAKT |
| | | YETTLEKCCAAADPHECYAKVFDEFKPLVEEPQNLIKQNCELFE |
| | | QLGEYKFQNALLVRYTKKVPQVSTPTLVEVSRNLGKVGSKCCKH |
| | | PEAKRMPCAEDYLSVVLNQLCVLHEKTPVSDRVTKCCTESLVNR |
| | | RPCFSALEVDETYVPKEFNAETFTFHADICTLSEKERQIKKQTA |
| | | LVELVKHKPKATKEQLKAVMDDFAAFVEKCCKADDKETCFAEEG |
| | | KKLVAASQAALGLEPKSCDKTHTCPPCPAPELLGGPSVFLFPPK |
| | | PKDTLMISRTPEVTCVVVDVSHEDPEVKENWYVDGVEVHNAKTK |
| | | PREEQYNSTYRVVSVLTVLHQDWLNGKEYKCKVSNKALPAPIEK |
| | | TISKAKGQPREPQVYTLPPSRDELTKNQVSLTCLVKGFYPSDIA |
| | | VEWESNGQPENNYKTTPPVLDSDGSFFLYSKLTVDKSRWQQGNV |
| | | FSCSVMHEALHNHYTQKSLSLSPGK |
| 5 | huG1Fc YTEKF- | EPKSCDKTHTCPPCPAPELLGGPSVFLFPPKPKDTLYITREPEV |
| | HSA | TCVVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNY |
| | | KTTPPVLDSDGSFFLYSKLTVDKSRWQQGNVFSCSVMHEALKFH |
| | | YTQKSLSLSPGKDAHKSEVAHRFKDLGEENFKALVLIAFAQYLQ |
| | | QCPFEDHVKLVNEVTEFAKTCVADESAENCDKSLHTLFGDKLCT |
| | | VATLRETYGEMADCCAKQEPERNECFLQHKDDNPNLPRLVRPEV |
| | | DVMCTAFHDNEETFLKKYLYEIARRHPYFYAPELLFFAKRYKAA |
| | | FTECCQAADKAACLLPKLDELRDEGKASSAKQRLKCASLQKFGE |
| | | RAFKAWAVARLSQRFPKAEFAEVSKLVTDLTKVHTECCHGDLLE |
| | | CADDRADLAKYICENQDSISSKLKECCEKPLLEKSHCIAEVEND |
| | | EMPADLPSLAADFVESKDVCKNYAEAKDVFLGMFLYEYARRHPD |
| | | YSVVLLLRLAKTYETTLEKCCAAADPHECYAKVEDEFKPLVEEP |
| | | QNLIKQNCELFEQLGEYKFQNALLVRYTKKVPQVSTPTLVEVSR |
| | | NLGKVGSKCCKHPEAKRMPCAEDYLSVVLNQLCVLHEKTPVSDR |
| | | VTKCCTESLVNRRPCFSALEVDETYVPKEFNAETFTFHADICTL |
| | | SEKERQIKKQTALVELVKHKPKATKEQLKAVMDDFAAFVEKCCK |
| | | ADDKETCFAEEGKKLVAASQAALGL |
| 6 | HSA- | DAHKSEVAHRFKDLGEENFKALVLIAFAQYLQQCPFEDHVKLVN |
| | huG1FcYTEKF | EVTEFAKTCVADESAENCDKSLHTLFGDKLCTVATLRETYGEMA |
| | | DCCAKQEPERNECFLQHKDDNPNLPRLVRPEVDVMCTAFHDNEE |
| | | TFLKKYLYEIARRHPYFYAPELLFFAKRYKAAFTECCQAADKAA |
| | | CLLPKLDELRDEGKASSAKQRLKCASLQKFGERAFKAWAVARLS |
| | | QRFPKAEFAEVSKLVTDLTKVHTECCHGDLLECADDRADLAKYI |
| | | CENQDSISSKLKECCEKPLLEKSHCIAEVENDEMPADLPSLAAD |
| | | FVESKDVCKNYAEAKDVFLGMFLYEYARRHPDYSVVLLLRLAKT |
| | | YETTLEKCCAAADPHECYAKVFDEFKPLVEEPQNLIKQNCELFE |
| | | QLGEYKFQNALLVRYTKKVPQVSTPTLVEVSRNLGKVGSKCCKH |
| | | PEAKRMPCAEDYLSVVLNQLCVLHEKTPVSDRVTKCCTESLVNR |
| | | RPCFSALEVDETYVPKEFNAETFTFHADICTLSEKERQIKKQTA |
| | | LVELVKHKPKATKEQLKAVMDDFAAFVEKCCKADDKETCFAEEG |
| | | KKLVAASQAALGLEPKSCDKTHTCPPCPAPELLGGPSVFLFPPK |
| | | PKDTLYITREPEVTCVVVDVSHEDPEVKENWYVDGVEVHNAKTK |
| | | PREEQYNSTYRVVSVLTVLHQDWLNGKEYKCKVSNKALPAPIEK |
| | | TISKAKGQPREPQVYTLPPSRDELTKNQVSLTCLVKGFYPSDIA |
| | | VEWESNGQPENNYKTTPPVLDSDGSFFLYSKLTVDKSRWQQGNV |
| | | FSCSVMHEALKFHYTQKSLSLSPGK |
| 7 | HSA(H464Q)- | DAHKSEVAHRFKDLGEENFKALVLIAFAQYLQQCPFEDHVKLVN |
| | huG1FcYTEKF | EVTEFAKTCVADESAENCDKSLHTLFGDKLCTVATLRETYGEMA |
| | | DCCAKQEPERNECFLQHKDDNPNLPRLVRPEVDVMCTAFHDNEE |
| | | TFLKKYLYEIARRHPYFYAPELLFFAKRYKAAFTECCQAADKAA |
| | | CLLPKLDELRDEGKASSAKQRLKCASLQKFGERAFKAWAVARLS |
| | | QRFPKAEFAEVSKLVTDLTKVHTECCHGDLLECADDRADLAKYI |
| | | CENQDSISSKLKECCEKPLLEKSHCIAEVENDEMPADLPSLAAD |
| | | FVESKDVCKNYAEAKDVFLGMFLYEYARRHPDYSVVLLLRLAKT |
| | | YETTLEKCCAAADPHECYAKVFDEFKPLVEEPQNLIKQNCELFE |
| | | QLGEYKFQNALLVRYTKKVPQVSTPTLVEVSRNLGKVGSKCCKH |
| | | PEAKRMPCAEDYLSVVLNQLCVLQEKTPVSDRVTKCCTESLVNR |
| | | RPCFSALEVDETYVPKEFNAETFTFHADICTLSEKERQIKKQTA |
| | | LVELVKHKPKATKEQLKAVMDDFAAFVEKCCKADDKETCFAEEG |
| | | KKLVAASQAALGLEPKSCDKTHTCPPCPAPELLGGPSVFLFPPK |
| | | PKDTLYITREPEVTCVVVDVSHEDPEVKFNWYVDGVEVHNAKTK |
| | | PREEQYNSTYRVVSVLTVLHQDWLNGKEYKCKVSNKALPAPIEK |
| | | TISKAKGQPREPQVYTLPPSRDELTKNQVSLTCLVKGFYPSDIA |
| | | VEWESNGQPENNYKTTPPVLDSDGSFFLYSKLTVDKSRWQQGNV |
| | | FSCSVMHEALKFHYTQKSLSLSPGK |
| 8 | huG1Fc YTEKF- | EPKSCDKTHTCPPCPAPELLGGPSVFLFPPKPKDTLYITREPEV |
| | HSA(H464Q) | TCVVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNY |
| | | KTTPPVLDSDGSFFLYSKLTVDKSRWQQGNVFSCSVMHEALKFH |
| | | YTQKSLSLSPGKDAHKSEVAHRFKDLGEENFKALVLIAFAQYLQ |
| | | QCPFEDHVKLVNEVTEFAKTCVADESAENCDKSLHTLFGDKLCT |
| | | VATLRETYGEMADCCAKQEPERNECFLQHKDDNPNLPRLVRPEV |
| | | DVMCTAFHDNEETFLKKYLYEIARRHPYFYAPELLFFAKRYKAA |
| | | FTECCQAADKAACLLPKLDELRDEGKASSAKQRLKCASLQKFGE |
| | | RAFKAWAVARLSQRFPKAEFAEVSKLVTDLTKVHTECCHGDLLE |
| | | CADDRADLAKYICENQDSISSKLKECCEKPLLEKSHCIAEVEND |
| | | EMPADLPSLAADFVESKDVCKNYAEAKDVFLGMFLYEYARRHPD |
| | | YSVVLLLRLAKTYETTLEKCCAAADPHECYAKVFDEFKPLVEEP |
| | | QNLIKQNCELFEQLGEYKFQNALLVRYTKKVPQVSTPTLVEVSR |
| | | NLGKVGSKCCKHPEAKRMPCAEDYLSVVLNQLCVLQEKTPVSDR |
| | | VTKCCTESLVNRRPCFSALEVDETYVPKEFNAETFTFHADICTL |
| | | SEKERQIKKQTALVELVKHKPKATKEQLKAVMDDFAAFVEKCCK |
| | | ADDKETCFAEEGKKLVAASQAALGL |
| 9 | huG1FcYPY-HSA | EPKSCDKTHTCPPCPAPELLGGPSVFLFPPKPKDTLYISRTPEV |
| | | TCVVVDVSHEDPEVKENWYVDGVEVHNAKTKPREEQYNSTYRVV |
| | | SVLTPLHQDWLNGKEYKCKVSNKALPAPIEKTISKAKGQPREPQ |
| | | VYTLPPSRDELTKNQVSLTCLVKGFYPSDIAVEWESNGQPENNY |
| | | KTTPPVLDSDGSFFLYSKLTVDKSRWQQGNVFSCSVMHEALHYH |
| | | YTQKSLSLSPGKDAHKSEVAHRFKDLGEENFKALVLIAFAQYLQ |
| | | QCPFEDHVKLVNEVTEFAKTCVADESAENCDKSLHTLFGDKLCT |
| | | VATLRETYGEMADCCAKQEPERNECFLQHKDDNPNLPRLVRPEV |
| | | DVMCTAFHDNEETFLKKYLYEIARRHPYFYAPELLFFAKRYKAA |
| | | FTECCQAADKAACLLPKLDELRDEGKASSAKQRLKCASLQKFGE |
| | | RAFKAWAVARLSQRFPKAEFAEVSKLVTDLTKVHTECCHGDLLE |
| | | CADDRADLAKYICENQDSISSKLKECCEKPLLEKSHCIAEVEND |
| | | EMPADLPSLAADFVESKDVCKNYAEAKDVFLGMFLYEYARRHPD |
| | | YSVVLLLRLAKTYETTLEKCCAAADPHECYAKVFDEFKPLVEEP |
| | | QNLIKQNCELFEQLGEYKFQNALLVRYTKKVPQVSTPTLVEVSR |
| | | NLGKVGSKCCKHPEAKRMPCAEDYLSVVLNQLCVLHEKTPVSDR |
| | | VTKCCTESLVNRRPCFSALEVDETYVPKEFNAETFTFHADICTL |
| | | SEKERQIKKQTALVELVKHKPKATKEQLKAVMDDFAAFVEKCCK |
| | | ADDKETCFAEEGKKLVAASQAALGL |
| 10 | HSA-huG1FcYPY | DAHKSEVAHRFKDLGEENFKALVLIAFAQYLQQCPFEDHVKLVN |
| | | EVTEFAKTCVADESAENCDKSLHTLFGDKLCTVATLRETYGEMA |
| | | DCCAKQEPERNECFLQHKDDNPNLPRLVRPEVDVMCTAFHDNEE |
| | | TFLKKYLYEIARRHPYFYAPELLFFAKRYKAAFTECCQAADKAA |
| | | CLLPKLDELRDEGKASSAKQRLKCASLQKFGERAFKAWAVARLS |
| | | QRFPKAEFAEVSKLVTDLTKVHTECCHGDLLECADDRADLAKYI |
| | | CENQDSISSKLKECCEKPLLEKSHCIAEVENDEMPADLPSLAAD |
| | | FVESKDVCKNYAEAKDVFLGMFLYEYARRHPDYSVVLLLRLAKT |
| | | YETTLEKCCAAADPHECYAKVFDEFKPLVEEPQNLIKQNCELFE |
| | | QLGEYKFQNALLVRYTKKVPQVSTPTLVEVSRNLGKVGSKCCKH |
| | | PEAKRMPCAEDYLSVVLNQLCVLHEKTPVSDRVTKCCTESLVNR |
| | | RPCFSALEVDETYVPKEFNAETFTFHADICTLSEKERQIKKQTA |
| | | LVELVKHKPKATKEQLKAVMDDFAAFVEKCCKADDKETCFAEEG |
| | | KKLVAASQAALGLEPKSCDKTHTCPPCPAPELLGGPSVFLFPPK |
| | | PKDTLYISRTPEVTCVVVDVSHEDPEVKENWYVDGVEVHNAKTK |
| | | PREEQYNSTYRVVSVLTPLHQDWLNGKEYKCKVSNKALPAPIEK |
| | | TISKAKGQPREPQVYTLPPSRDELTKNQVSLTCLVKGFYPSDIA |
| | | VEWESNGQPENNYKTTPPVLDSDGSFFLYSKLTVDKSRWQQGNV |
| | | FSCSVMHEALHYHYTQKSLSLSPGK |
| 11 | huG1FcYPY- | EPKSCDKTHTCPPCPAPELLGGPSVFLFPPKPKDTLYISRTPEV |
| | HSA(H464Q) | TCVVVDVSHEDPEVKENWYVDGVEVHNAKTKPREEQYNSTYRVV |
| | | SVLTPLHQDWLNGKEYKCKVSNKALPAPIEKTISKAKGQPREPQ |
| | | VYTLPPSRDELTKNQVSLTCLVKGFYPSDIAVEWESNGQPENNY |
| | | KTTPPVLDSDGSFFLYSKLTVDKSRWQQGNVFSCSVMHEALHYH |
| | | YTQKSLSLSPGKDAHKSEVAHRFKDLGEENFKALVLIAFAQYLQ |
| | | QCPFEDHVKLVNEVTEFAKTCVADESAENCDKSLHTLFGDKLCT |
| | | VATLRETYGEMADCCAKQEPERNECFLQHKDDNPNLPRLVRPEV |
| | | DVMCTAFHDNEETFLKKYLYEIARRHPYFYAPELLFFAKRYKAA |
| | | FTECCQAADKAACLLPKLDELRDEGKASSAKQRLKCASLQKFGE |
| | | RAFKAWAVARLSQRFPKAEFAEVSKLVTDLTKVHTECCHGDLLE |
| | | CADDRADLAKYICENQDSISSKLKECCEKPLLEKSHCIAEVEND |
| | | EMPADLPSLAADFVESKDVCKNYAEAKDVFLGMFLYEYARRHPD |
| | | YSVVLLLRLAKTYETTLEKCCAAADPHECYAKVFDEFKPLVEEP |
| | | QNLIKQNCELFEQLGEYKFQNALLVRYTKKVPQVSTPTLVEVSR |
| | | NLGKVGSKCCKHPEAKRMPCAEDYLSVVLNQLCVLQEKTPVSDR |
| | | VTKCCTESLVNRRPCFSALEVDETYVPKEFNAETFTFHADICTL |
| | | SEKERQIKKQTALVELVKHKPKATKEQLKAVMDDFAAFVEKCCK |
| | | ADDKETCFAEEGKKLVAASQAALGL |
| 12 | HSA(H464Q)- | DAHKSEVAHRFKDLGEENFKALVLIAFAQYLQQCPFEDHVKLVN |
| | huG1FcYPY | EVTEFAKTCVADESAENCDKSLHTLFGDKLCTVATLRETYGEMA |
| | | DCCAKQEPERNECFLQHKDDNPNLPRLVRPEVDVMCTAFHDNEE |
| | | TFLKKYLYEIARRHPYFYAPELLFFAKRYKAAFTECCQAADKAA |
| | | CLLPKLDELRDEGKASSAKQRLKCASLQKFGERAFKAWAVARLS |
| | | QRFPKAEFAEVSKLVTDLTKVHTECCHGDLLECADDRADLAKYI |
| | | CENQDSISSKLKECCEKPLLEKSHCIAEVENDEMPADLPSLAAD |
| | | FVESKDVCKNYAEAKDVFLGMFLYEYARRHPDYSVVLLLRLAKT |
| | | YETTLEKCCAAADPHECYAKVFDEFKPLVEEPQNLIKQNCELFE |
| | | QLGEYKFQNALLVRYTKKVPQVSTPTLVEVSRNLGKVGSKCCKH |
| | | PEAKRMPCAEDYLSVVLNQLCVLQEKTPVSDRVTKCCTESLVNR |
| | | RPCFSALEVDETYVPKEFNAETFTFHADICTLSEKERQIKKQTA |
| | | LVELVKHKPKATKEQLKAVMDDFAAFVEKCCKADDKETCFAEEG |
| | | KKLVAASQAALGLEPKSCDKTHTCPPCPAPELLGGPSVELFPPK |
| | | PKDTLYISRTPEVTCVVVDVSHEDPEVKFNWYVDGVEVHNAKTK |
| | | PREEQYNSTYRVVSVLTPLHQDWLNGKEYKCKVSNKALPAPIEK |
| | | TISKAKGQPREPQVYTLPPSRDELTKNQVSLTCLVKGFYPSDIA |
| | | VEWESNGQPENNYKTTPPVLDSDGSFFLYSKLTVDKSRWQQGNV |
| | | FSCSVMHEALHYHYTQKSLSLSPGK |
| 13 | huG1FcYTEKF | EPKSCDKTHTCPPCPAPELLGGPSVFLFPPKPKDTLYITREPEV |
| | | TCVVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNY |
| | | KTTPPVLDSDGSFFLYSKLTVDKSRWQQGNVFSCSVMHEALKFH |
| | | YTQKSLSLSPGK |

1. A neonatal crystallizable fragment receptor (FcRn) antagonist comprising:
(i) an immunoglobulin Fc domain or fragment thereof capable of binding to a FcRn; and
(ii) at least one albumin or fragment thereof capable of extending the half-life of the FcRn antagonist compared to the half-life of the immunoglobulin Fc domain or the fragment thereof.

2. The FcRn antagonist of claim 1, wherein the antagonist has a longer serum half-life compared to the immunoglobulin Fc domain or fragment thereof alone.

3. The FcRn antagonist of claim 1 or 2, wherein the antagonist binds to human FcRn at neutral pH with an affinity constant (K_{D}) of at least 500 nM and/or to human FcRn at acidic pH with a K_{D} of at least 100 nM.

4. The FcRn antagonist of any one of claims 1 to 3, wherein the albumin or fragment thereof is a human albumin variant or fragment thereof.

5. The FcRn antagonist of claim 4, wherein the albumin variant or fragment thereof binds with reduced affinity to FcRn compared to an albumin set forth in SEQ ID NO: 1.

6. The FcRn antagonist of claim 5, wherein the binding affinity is measured at neutral and/or acidic pH.

7. The FcRn antagonist of any one of claims 4 to 6, wherein the albumin variant or fragment thereof binds to human FcRn at neutral pH or pH 6.0 with a K_{D} of greater than 10 μM.

8. The FcRn antagonist of any one of claims 4 to 7, wherein the albumin variant or fragment thereof comprises one or more amino acid substitutions selected from the group consisting of:
(i) glutamine substituted for histidine at a position corresponding to amino acid 464 of SEQ ID NO: 1;
(ii) tryptophan substituted for threonine at a position corresponding to amino acid 422 of SEQ ID NO: 1;
(iii) glutamine substituted for histidine at a position corresponding to amino acid 510 of SEQ ID NO: 1;
(iv) phenylalanine substituted for histidine at a position corresponding to amino acid 535 of SEQ ID NO: 1; and
(v) combinations thereof.

9. The FcRn antagonist of any one of claims 4 to 8, wherein the albumin variant or fragment thereof comprises:
(i) glutamine substituted for histidine at a position corresponding to amino acid 464 of SEQ ID NO: 1; or
(ii) phenylalanine substituted for histidine at a position corresponding to amino acid 535 of SEQ ID NO: 1; or
(iii) tryptophan substituted for threonine at a position corresponding to amino acid 422 of SEQ ID NO: 1, and glutamine substituted for histidine at a position corresponding to amino acid 464 of SEQ ID NO: 1; or
(iv) glutamine substituted for histidine at a position corresponding to amino acid 464 of SEQ ID NO: 1 and phenylalanine substituted for histidine at a position corresponding to amino acid 535 of SEQ ID NO: 1.

10. The FcRn antagonist of any one of claims 1 to 9, wherein the immunoglobulin Fc domain or fragment thereof is an Fc domain variant or fragment thereof.

11. The FcRn antagonist of claim 10, wherein the Fc domain variant or fragment thereof is an IgG1 Fc domain variant or fragment thereof.

12. The FcRn antagonist of claim 10 or 11, wherein the IgG1 Fc domain variant or fragment thereof binds with increased affinity to human FcRn compared to an IgG1 Fc domain set forth in SEQ ID NO: 2.

13. The FcRn antagonist of claim 12, wherein the binding affinity is measured at neutral and/or acidic pH.

14. The FcRn antagonist of any one of claims 10 to 13, wherein the Fc domain variant or fragment thereof binds to human FcRn at neutral pH with a K_{D} of at least 10 μM.

15. The FcRn antagonist of any one of claims 10 to 13, wherein the Fc domain variant or fragment thereof comprises one or more amino acid substitutions selected from the group consisting of:
(i) tyrosine substituted for methionine at a position corresponding to amino acid 252 according to the EU numbering system;
(ii) threonine substituted for serine at a position corresponding to amino acid 254 according to the EU numbering system;
(iii) glutamic acid substituted for threonine at a position corresponding to amino acid 256 according to the EU numbering system;
(iv) glutamic acid substituted for asparagine at a position corresponding to amino acid 286 according to the EU numbering system;
(v) proline substituted for valine at a position corresponding to amino acid 308 according to the EU numbering system;
(vi) lysine substituted for histidine at a position corresponding to amino acid 433 according to the EU numbering system;
(vii) tyrosine substituted for asparagine at a position corresponding to amino acid 434 according to the EU numbering system;
(viii) phenylalanine substituted for asparagine at a position corresponding to amino acid 434 according to the EU numbering system; and
(ix) combinations thereof.

16. The FcRn antagonist of any one of claims 10 to 15, wherein the Fc domain variant or fragment thereof comprises:
(i) tyrosine substituted for methionine at a position corresponding to amino acid 252 according to the EU numbering system, proline substituted for valine at a position corresponding to amino acid 308 according to the EU numbering system and tyrosine substituted for asparagine at a position corresponding to amino acid 434 according to the EU numbering system; or
(ii) tyrosine substituted for methionine at a position corresponding to amino acid 252 according to the EU numbering system, threonine substituted for serine at a position corresponding to amino acid 254 according to the EU numbering system and glutamic acid substituted for threonine at a position corresponding to amino acid 256 according to the EU numbering system; or
(iii) tyrosine substituted for methionine at a position corresponding to amino acid 252 according to the EU numbering system, glutamic acid substituted for asparagine at a position corresponding to amino acid 286 according to the EU numbering system and tyrosine substituted for asparagine at a position corresponding to amino acid 434 according to the EU numbering system; or
(iv) tyrosine substituted for methionine at a position corresponding to amino acid 252 according to the EU numbering system threonine substituted for serine at a position corresponding to amino acid 254 according to the EU numbering system, glutamic acid substituted for threonine at a position corresponding to amino acid 256 according to the EU numbering system, lysine substituted for histidine at a position corresponding to amino acid 433 according to the EU numbering system and phenylalanine substituted for asparagine at a position corresponding to amino acid 434 according to the EU numbering system.

17. The FcRn antagonist of any one of claims 1 to 16, wherein:
(i) the Fc domain comprises tyrosine substituted for methionine at a position corresponding to amino acid 252 according to the EU numbering system, proline substituted for valine at a position corresponding to amino acid 308 according to the EU numbering system and tyrosine substituted for asparagine at a position corresponding to amino acid 434 according to the EU numbering system; and
(ii) the albumin comprises glutamine substituted for histidine at a position corresponding to amino acid 464 according to the EU numbering system.

18. The FcRn antagonist of any one of claims 1 to 17, wherein the Fc domain or fragment thereof is indirectly linked to the albumin or fragment thereof via a linker.

19. The FcRn antagonist of claim 18, wherein the linker is a peptide linker comprising between 2 and 31 amino acids in length.

20. The FcRn antagonist of any one of claims 1 to 17, wherein Fc domain or fragment thereof is directly linked to an albumin or fragment thereof.

21. The FcRn antagonist of any one of claims 1 to 20, wherein the C-terminus of the Fc domain or fragment thereof is indirectly or directly linked to the N-terminus of an albumin or fragment thereof.

22. The FcRn antagonist of any one of claims 1 to 21, wherein the FcRn antagonist comprises two or more albumins or fragments thereof.

23. A FcRn antagonist comprising:
(i) an immunoglobulin Fc domain or fragment thereof comprising tyrosine substituted for methionine at a position corresponding to amino acid 252 according to the EU numbering system, proline substituted for valine at a position corresponding to amino acid 308 according to the EU numbering system and tyrosine substituted for asparagine at a position corresponding to amino acid 434 according to the EU numbering system; and
(ii) an albumin or fragment thereof comprising glutamine substituted for histidine at a position corresponding to amino acid 464 of SEQ ID NO: 1,
wherein the C-terminus of the Fc domain or fragment thereof is indirectly or directly linked to the N-terminus of the albumin or fragment thereof.

24. A composition comprising the FcRn antagonist of any one of claims 1 to 22 and a pharmaceutically acceptable carrier.

25. The FcRn antagonist of any one of claims 1 to 23, or the composition of claim 24, for use in reducing circulating Fc-containing proteins and/or antibodies in a subject in need thereof.

26. The FcRn antagonist of any one of claims 1 to 23, or the composition of claim 24, for use in treating or preventing progression of an antibody-mediated disorder in a subject in need thereof.

27. A method of reducing circulating antibodies in a subject in need thereof, the method comprising administering the FcRn antagonist of any one of claims 1 to 23, or the composition of claim 24.

28. A method of treating or preventing progression of an antibody-mediated disorder in a subject in need thereof, the method comprising administering the FcRn antagonist of any one of claims 1 to 23, or the composition of claim 24.

29. Use of the FcRn antagonist of any one of claims 1 to 23, or the composition of claim 24 in the manufacture of a medicament for reducing circulating antibodies in a subject in need thereof.

30. Use of the FcRn antagonist of any one of claims 1 to 23, or the composition of claim 24 in the manufacture of a medicament for treating or preventing progression of an antibody-mediated disorder in a subject in need thereof.

31. The FcRn antagonist of claim 25 or 26, the method of claim 27 or 28, or the use of claim 29 or 30, wherein the subject is suffering from an autoimmune disease, has developed anti-drug antibodies or is at risk of developing anti-drug antibodies.

32. The FcRn antagonist of any one of claim 25, 26 or 31, the method of any one of claim 27, 28 or 31 or the use of any one of claims 29 to 31, wherein the FcRn antagonist is administered in an amount effective to:
(i) reduce endogenous IgG levels by at least 1× (compared to in the absence of FcRn antagonist administration); and/or
(ii) reduce endogenous albumin levels by no more than 20% (compared to in the absence of FcRn antagonist administration).

33. The FcRn antagonist of any one of claim 25, 26, 31 or 32, the method of any one of claim 27, 28, 31 or 32, or the use of any one of claims 29 to 32, wherein the FcRn antagonist antagonizes IgG recycling, however does not substantially antagonize albumin recycling.

34. The FcRn antagonist of any one of claim 25, 26, 31 or 32, the method of any one of claim 27, 28, 31 or 32, or the use of any one of claims 29 to 32, wherein administration of the FcRn antagonist does not induce dyslipidemia and/or induces a smaller increase in serum cholesterol levels compared to the level observed following administration of a FcRn antagonist comprising wild-type human albumin.

35. The FcRn antagonist of any one of claims 25, 26, 31 to 32, the method of any one of claims 27, 28, 31 to 32, or the use of any one of claims 29 to 32, wherein administration of the FcRn antagonist cross-links cell surface FcRn at a lower level than a FcRn antagonist comprising wild-type human albumin.

36. The FcRn antagonist of any one of claims 25, 26, or 31 to 35, the method of any one of claims 27, 28 or 31 to 35, or the use of any one of claims 29 to 35, wherein the subject has received, is receiving, or will receive an additional therapy.

37. The FcRn antagonist, the method or the use of claim 36, wherein the additional therapy is a steroidal immune modulator, plasmapheresis and/or IVIg therapy.

38. A kit for use in reducing circulating autoantibodies in a subject in need thereof, the kit comprising:
(i) at least one FcRn antagonist of any one of claims 1 to 23, or pharmaceutical composition of claim 24;
(ii) instructions for using the kit in reducing circulating autoantibodies in the subject; and
(iii) optionally, at least one additional therapy.

39. A kit for treating or preventing progression of an antibody-mediated disorder in a subject in need thereof, the kit comprising:
(i) at least one FcRn antagonist of any one of claims 1 to 23, or pharmaceutical composition of claim 24;
(ii) instructions for using the kit in treating or preventing progression of an antibody-mediated disorder in the subject; and
(iii) optionally, at least one additional therapy.


SEQUENCE LISTING (13 sequences)

SEQ ID NO: 1 - protein, 585 aa, Homo sapiens [sequence omitted]

SEQ ID NO: 2 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 817 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 4 - protein, 817 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 817 aa, synthetic construct
EPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGKDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 6 - protein, 817 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 7 - protein, 817 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLQEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 8 - protein, 817 aa, synthetic construct
EPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGKDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLQEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 9 - protein, 817 aa, synthetic construct
EPKSCDKTHTCPPCPAPELLGGPSVFLFPPKPKDTLYISRTPEVTCVVVDVSHEDPEVKFNWYVDGVEVHNAKTKPREEQYNSTYRVVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHYHYTQKSLSLSPGKDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 10 - protein, 817 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLEPKSCDKTHTCPPCPAPELLGGPSVFLFPPKPKDTLYISRTPEVTCVVVDVSHEDPEVKFNWYVDGVEVHNAKTKPREEQYNSTYRVVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHYHYTQKSLSLSPGK

SEQ ID NO: 11 - protein, 817 aa, synthetic construct
EPKSCDKTHTCPPCPAPELLGGPSVFLFPPKPKDTLYISRTPEVTCVVVDVSHEDPEVKFNWYVDGVEVHNAKTKPREEQYNSTYRVVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHYHYTQKSLSLSPGKDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLQEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 12 - protein, 817 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLQEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLEPKSCDKTHTCPPCPAPELLGGPSVFLFPPKPKDTLYISRTPEVTCVVVDVSHEDPEVKFNWYVDGVEVHNAKTKPREEQYNSTYRVVSVLTPLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHYHYTQKSLSLSPGK

SEQ ID NO: 13 - protein, 232 aa, synthetic construct
EPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK